(19) United States
(12) Patent Application Publication
(10) Pub. No.: US 2023/0051872 A1
(43) Pub. Date: Feb. 16, 2023

(54) Multivalent D-Peptidic Compounds for Target Proteins

(71) Applicant: Reflexion Pharmaceuticals, Inc., Incline Village, NV (US)

(72) Inventors: Paul Marinec, Incline Village, NV (US); Kyle Landgraf, Incline Village, NV (US); Dana Ault-Riche, Incline Village, NV (US)

(21) Appl. No.: 17/441,645

(22) PCT Filed: Mar. 20, 2020
(86) PCT No.: PCT/US2020/024056

§ 371 Date: Sep. 21, 2021

Related U.S. Application Data
(60) Provisional application No. 62/822,241, filed on Mar. 22, 2019.

(51) Int. Cl. C07K 16/46

(52) U.S. Cl. CPC C07K 16/468; C07K 2317/35; C07K 2317/76; C07K 2318/20; C07K 2317/92; C07K 2317/31

(57) ABSTRACT

Multivalent D-peptidic compounds that specifically bind to a target protein are provided. The multivalent D-peptidic compounds can include two or more distinct variant D-peptidic domains connected via linking components. The D-peptidic compounds can include multiple distinct domains that specifically bind to different binding sites on a target protein to provide for high affinity binding to, and potent activity against, the target protein. D-peptidic variant GA and Z domain polypeptides are also provided, which polypeptides have specificity-determining motifs (SDM) for specific binding to a target protein, such as VEGF-A or PD-1. In some embodiments where the target protein is homodimeric (e.g., VEGF-A, PD-1), the D-peptidic compounds may be similarly dimeric, and include a dimer of multivalent (e.g., bivalent) D-peptidic compounds. Methods for using the compounds are provided, including methods for treating a disease or condition associated with a target protein in a subject.

### CROSS-REFERENCE TO RELATED APPLICATIONS

This application claims the benefit of U.S. Provisional Patent Application No. 62/822,241, filed Mar. 22, 2019, which application is incorporated herein by reference in its entirety.

### INTRODUCTION

Mirror image phage display is a method for identifying D-polypeptide ligands that bind to a native target protein that involves initial screening of a phage display library of L-polypeptides against the chemically synthesized D-enantiomer of the native target protein. See Kim et al., “Identification of D-Peptide Ligands Through Mirror Image Phage Display”, Science, 271, 1854-1857 (1996)). The resulting ligands identified through the screening can then be prepared chemically in D-enantiomeric form using conventional solid phase peptide synthesis methods and D-amino acid building blocks.

D-proteins that specifically bind therapeutic target proteins with high affinity and activity in vivo are of great interest.

### SUMMARY

Multivalent D-peptidic compounds that specifically bind to a target protein are provided. The multivalent D-peptidic compounds can include two or more distinct variant D-peptidic domains connected via linking components. The multivalent (e.g., bivalent, trivalent, tetravalent, etc.) D-peptidic compounds can include multiple distinct domains that specifically bind to different binding sites on a target protein to provide for high affinity binding to, and potent activity against, the target protein. D-peptidic variant GA and Z domain polypeptides that find use in the multivalent compounds are also provided, which polypeptides have specificity-determining motifs (SDM) for specific binding to a target protein, such as PD-1. In some embodiments where the target protein is homodimeric, the D-peptidic compounds may be similarly dimeric, and include a dimer of multivalent (e.g., bivalent) D-peptidic compounds. The subject D-peptidic compounds find use in a variety of applications in which specific binding to a target is desired. Methods for using the compounds are provided, including methods for treating a disease or condition associated with a target protein in a subject.

### BRIEF DESCRIPTION OF THE DRAWINGS

FIG. 1A-1B show depictions of the structure (FIG. 1A) and sequence (FIG. 1B) of a phage display library based on a parent Z domain scaffold. Ten positions (X) were selected within helix 1 to helix 2 of the Z domain for randomization using kunkel mutagenesis with trinucleotide codons representing all the amino acids except cysteine.

FIG. 2A-2B show depictions of the structure (FIG. 2A) and sequence (FIG. 2B) of a phage display library based on a parent GA domain scaffold. Eleven positions (X) were selected within helix 2 to helix 3 of the GA domain scaffold for randomization using kunkel mutagenesis with trinucleotide codons representing all amino acids except cysteine.

FIG. 3A-3D show the results of mirror image phage display screening for binding to the PD-1 target construct using a GA domain phage display library. FIG. 3A shows a consensus sequence logo that provides for binding to PD-1. FIG. 3B shows selected variant GA domain sequences of interest (SEQ ID NOs: 32-35) with their D-peptidic binding affinities for native L-PD-1. NB refers to non-binding. FIG. 3C shows the structure of 977296 in isolation looking at the PD-1 binding face of the compound with the variant amino acid residues selected from the GA domain library shown in red. FIG. 3D shows an expanded view of the protein to protein contacts (top panel) and the binding site on PD-1 (bottom panel) of compound 977296 including the configuration of variant amino acids in contact with the binding site (top panel).

FIG. 4A-4F show the results of mirror image phage display screening for binding to the PD-1 target construct using a Z domain phage display library. FIG. 4A shows a consensus sequence logo that provides for binding to PD-1. FIG. 4B shows selected variant Z domain sequences of interest (SEQ ID NOs: 36-41) with binding affinities as measured for D-peptidic compounds binding to native L-PD-1. NB refers to non-binding. FIG. 4C shows the structure of 978064 in isolation looking at the PD-1 binding face of the compound with the variant amino acid residues selected from the Z domain library shown in red. FIG. 4D shows an expanded view of the protein to protein contacts (top panel) and the binding site on PD-1 (bottom panel) of compound 978064 including the configuration of variant amino acids in contact with the binding site (top panel). FIG. 4E shows an expanded view of the crystal structure of compound 978064 bound to PD-1, showing that although residues k4, f5, n6, k7 and i31 were close to the surface of PD-1 and capable of making some contacts with the target protein, these residues were potential sites for improvement of binding affinity.

FIGS. 4F-4G illustrate affinity maturation results of exemplary compound 978064. FIG. 4F shows a strong consensus sequence representative of the affinity maturation. FIG. 4G shows the sequences of compounds 981185, 981196 and 981187, and their binding affinities for PD-1 relative to the parent compound as measured using SPR.

FIG. 5 shows a representative surface plasmon resonance (SPR) sensorgram showing additive binding of compounds 977296 and 978064, indicating that compound 977296 (a variant GA domain compound) binds to a binding site on PD-1 that is non-overlapping and independent of the binding site of compound 978064 (variant Z domain compound).

FIG. 6 shows a graph measuring antagonism of PD-1 binding to PD-L1 for D-peptidic compounds 977296 and 978064 as compared to anti-PD-1 antagonist antibody nivolumab. Compound 977296 showed no detectable inhibition of PD-1 binding to PD-L1, indicating its binding site on PD-1 does not overlaps with the PD-L1 binding site of PD-1.

FIG. 7A-7B show two depictions of the X ray crystal structure of D-peptidic compounds 977296 and 978064 each bound to L-PD-1. FIG. 7A shows the two D-peptidic compounds bind to distinct and separate sites of L-PD-1. FIG. 7B shows the structure of FIG. 7A, where the D-peptidic compounds 977296 and 978064 are represented with a space filling model, overlaid with the structure of PD-L1 bound to PD-1 at its binding site. The overlay shows that D-peptidic compound 978064 directly overlaps with, and blocks binding of, PD-L1 to PD-1.

FIG. 8A-8C illustrate the structure based-design of a exemplary bivalent compounds, including compounds 977296 and 978064 conjugated to each other via N-terminal cysteine residues using a bis-maleimide PEG3, PEG6 or PEG8 linker (FIG. 8A). FIG. 8B illustrates the sequence of N-cysteine derived compounds 977296 and 978064 and identification of bivalent compounds 979821, 979820, and 979450 which exhibited >1,000-fold improvement in binding affinity for the conjugate over either parent compound as measured by SPR. Bivalent compounds 979821, 979820, and 979450 were prepared by linking 977296 and 978064 which were each modified to incorporate N-terminal cysteine residues and conjugating with Maleimide-PEGn-Maleimide bifunctional linker (shown as Mal-PEGn-Mal in the figure). FIG. 8C shows a schematic of an alternative bivalent compound conjugate design where the compound 978064 could be N-terminal truncated to the k4 residue and conjugated to the the N-terminal residue of compound 977296 via a linker of about 22 angstroms (e.g., a cysteine-Maleimide-PEGn-Maleimide-cysteine linker). One or more optional spacer residues (e.g., a, G and/or s residues) can also be incorporated between such a N-terminal cysteine residue and the Z or GA domain, e.g., as part of the linking component.

FIG. 9 shows a graph illustrating antagonism of PD-1 binding to PD-L1 for D-peptidic bivalent compounds 979821, 979820, and 979450 which exhibit comparable IC₅₀ values to the anti-PD-1 antagonist antibody nivolumab.

FIG. 10 shows a graph illustrating the results of a T-cell activation assay that measures blockade of the PD-1/PD-L1 pathway by bivalent compounds 979821, 979820, and 979450 as compared to the anti-PD-1 antagonist antibody nivolumab.

FIG. 11 shows a synthetic strategy for the total chemical synthesis of PD-1. Sequential native chemical ligation of four peptide segments was utilized to prepare the 165 amino acid PD-1 polypeptide chain in both L- and D-forms.

FIG. 12 shows LC/MS spectra for L-PD-1 following chemical synthesis and purification.

FIG. 13A shows titration of chemically synthesized and refolded L-PD-1 binding to nivolumab immobilized on an ELISA plate.

FIG. 13B shows SPR sensorgram of the association and dissociation reactions measured for titrations of nivolumab binding to refolded L-PD-1 on the sensor chip surface.

FIG. 14A shows Z domain scaffold sequence and phage library used for panning. Red X denotes the hard-randomized positions in the naïve library and red residues targeted for soft randomization during affinity maturation. Lowercase amino acids denote D-amino acids and the red lowercase D-amino acids represent selected mutations corresponding to binders.

FIG. 14B shows GA-domain scaffold sequence and phage library used for panning. Red X denotes the hard-randomized positions in the naïve library and red residues targeted for soft randomization during affinity maturation. Lowercase amino acids denote D-amino acids and the red lowercase D-amino acids represent selected mutations corresponding to binders.

FIG. 15 shows SPR sensorgrams of the association and dissociation reactions measured for titrations of RFX-978064 and RFX-977296 binding to PD-1-Fc on the sensor chip surface.

FIG. 16 shows a Table summarizing the SPR-derived kinetic binding parameters for D-proteins and nivolumab binding to PD-1-Fc.

FIG. 17 shows titrations of synthetic D-proteins RFX-977296 (grey filled circles) and RFX-978064 (open circles) in a PD-1 blocking ELISA showing antagonistic activity relative to nivolumab (black filled circles).

FIG. 18 shows a table summarizing the IC₅₀ values for exemplary D-peptidic compounds 977296, 978064 and 979261 versus nivolumab for blocking PD-1-Fc binding to PD-L1-Fc in an ELISA.

FIG. 19 shows SPR-based epitope mapping where 1 μM of RFX-977296 is used to saturate PD-1 on the chip surface. In the second association step, 1 μM of RFX-978064 is included with 1 μM of RFX-977296 and exhibits additive binding to PD-1, indicating the site for RFX-978064 is not blocked by RFX-977296.

FIG. 20 shows overview of x-ray crystal structure showing RFX-978064 (purple) and RFX-977296 (blue) bound to distinct, non-overlapping epitopes on PD-1.

FIG. 21 shows data collection and refinement statistics for x-ray crystal structure of PD-1/D-protein triple complex.

FIG. 22 shows interfacial D-amino acid side chains contacting PD-1 depicted for RFX-978064 with selected library residues (green) and original scaffold backbone residues (purple) within helix 1 and 2. PD-1 is shown with electrostatic surface potential to highlight positive (blue), negative (red), and neutral hydrophobic (white) contact sites.

FIG. 23A shows crystal structure of PD-1 (grey) in complex with PD-L1 (orange) (PDB code: 4ZQK) (22).

FIG. 23B shows overlay of RFX-977296 and RFX-978064 on the PD-1/PD-L1 complex to demonstrate direct competition between RFX-978064 and PD-L1 as the mechanism for PD-1 inhibition.

FIG. 24 shows structural characterization of the PD-1 binding interface showing a conserved tryptophan residue from RFX-978064 (purple) binding in a hydrophobic pocket of PD-1 (grey), similar to its interaction with Tyrosine 123 of PD-L1 (orange) from a previously solved PD-1/PD-L1 structure (22).

FIG. 25 shows interfacial D-amino acid side chains contacting PD-1 depicted for RFX-977296 with selected library residues (green) and original scaffold backbone residues (blue) within helix 2 and 3. PD-1 is shown with electrostatic surface potential to highlight positive (blue), negative (red), and neutral hydrophobic (white) contact sites.

FIG. 26 shows structure of RFX-978064 (purple) bound to PD-1 (grey) showing seven residues (orange) in the helix 1-2 binding interface targeted for affinity maturation.

FIG. 27 shows SPR sensorgram of the association and dissociation reaction measured for titrations of RFX-979261 binding to PD-1-Fc on the sensor chip surface.

FIG. 28 shows titrations of the affinity matured D-protein RFX-979261 (grey filled circles) in the PD-1 blocking ELISA showing antagonistic activity relative to RFX-978064 (open circles) and nivolumab (black filled circles).

FIG. 29 shows structure of RFX-977296 (blue) bound to PD-1 (grey) showing the helix 2-3 binding interface and the nine residues selected for soft-randomization libraries.

FIG. 30 shows design of the heterodimeric RFX-979820 clasp showing N-terminal to N-terminal distance between RFX-977296 and RFX-978064 for maleimide conjugation of linker.

FIG. 31 shows full D-amino acid sequence for heterodimeric or bivalent D-peptidic compounds RFX-979820 (SEQ ID NO: 46), 979821 (SEQ ID NO: 45), 979450 (SEQ ID NO: 47), and 981851 (SEQ ID NO: 48). The compounds include N-terminal to N-terminal linkers including N-terminal addition of D-cysteine residues which are subsequently covalent linked using a bis-maleimide PEGn bifunctional linking moiety. This is depicted as “PEGn” in FIG. 31 where n is 6, 3, 8 or 6, respectively.

FIG. 32 shows Chemical synthesis scheme for the heterodimeric D-protein RFX-979820.

FIG. 33 shows SPR sensorgrams of the single-cycle association and dissociation reactions measured for RFX-979820, RFX-982007, and nivolumab binding to PD-1-Fc on the sensor chip surface.

FIG. 34 shows full D-amino acid sequence for trivalent D-protein RFX-982007 (SEQ ID NO: 50), 980861 (SEQ ID NO: 49), and 982864 (SEQ ID NO: 51). For compound RFX-980861 FIG. 35 shows a chemical synthesis scheme for the trimeric D-protein RFX-982007.

FIG. 36 shows titrations of the heterodimeric RFX-979820 (open squares) and the trimeric RFX-982007 (grey filled squares) in a PD-1 blocking ELISA showing antagonistic activity relative to nivolumab (black filled circles).

FIG. 37 shows table summarizing the IC₅₀ values for D-proteins and nivolumab blocking PD-1-Fc binding to nivolumab.

FIG. 38 shows titrations of RFX-979820 (open squares), and RFX-982007 (grey filled circles) in a T-cell activation assay showing dose-dependent activation of TCR signaling relative to nivolumab (black filled circles).

FIG. 39 shows a table summarizing the EC₅₀ values for D-proteins and nivolumab blocking PD-1 in a T-cell receptor activation assay.

FIG. 40 shows titrations of the trimeric RFX-982007 showing a dose-dependent increase in the proliferation of CD8⁺ T-cells in a CMV antigen recall assay relative to nivolumab, as well as dose-dependent increases in the production of the cytokines (E) TNF-α and (F) IFN-γ in a CMV antigen recall assay relative to nivolumab.

FIG. 41 shows titrations of the trimeric RFX-982007 showing a dose-dependent increase in the proliferation of CD4⁺ T-cells in a CMV antigen recall assay relative to nivolumab.

FIG. 42 shows titrations of the trimeric RFX-982007 showing a dose-dependent increase in the production of TNF-α in a CMV antigen recall assay relative to nivolumab.

FIG. 43 shows titrations of the trimeric RFX-982007 showing a dose-dependent increase in the production of IFN-γ in a CMV antigen recall assay relative to nivolumab.

FIG. 44A shows anti-drug antibodies measured in the serum of mice before and 21, 35, and 42 days after subcutaneous immunization with nivolumab using an ELISA for antigen-specific serum IgG.

FIG. 44B shows anti-drug antibodies measured in the serum of mice before and 21, 35, and 42 days after subcutaneous immunization with RFX-982007 using an ELISA for antigen-specific serum IgG.

FIG. 45 shows overlay of PD-1 backbone when bound to RFX-978064 with a previously solved PD-1 crystal structure (22) showing rearrangements in the FG and CC′loop of PD-1.

FIG. 46A shows cavities present in the RFX-978064/PD-1 binding interface (grey) can accommodate several sidechains of RFX-978064 (purple).

FIG. 46B shows PD-1 cavities that accommodate several sidechains of RFX-978064 (purple) are occluded when bound to PD-L1 (dark grey).

FIG. 47A shows solved x-ray crystal structure illustrating the binding site on PD-1 (grey) for nivolumab (fuschia).

FIG. 47B shows x-ray crystal structure of PD-1 bound to RFX-977296 and RFX-978064 illustrating RFX-978064 binds a similar epitope as nivolumab (fuschia).

FIG. 48A shows solved x-ray crystal structure illustrating the binding site on PD-1 (grey) for pembrolizumab (teal).

FIG. 48B shows x-ray crystal structure of PD-1 bound to RFX-977296 and RFX-978064 illustrating RFX-978064 binds a similar epitope as pembrolizumab (teal).

FIG. 49 shows x-ray crystal structure of PD-1 (grey) bound to RFX-977296 and RFX-978064 illustrating RFX-977296 partially overlaps with that of the anti-CD28 antibody NB01a (see circle).

FIG. 50 shows a SDM for a D-peptidic GA domain that binds PD-1.

FIG. 51 shows a SDM for a D-peptidic Z domain that binds PD-1.

### DETAILED DESCRIPTION

### Multivalent D-Peptidic Binding Compounds

As summarized above, aspects of this disclosure include multivalent D-peptidic compounds that specifically bind with high affinity to a target protein. This disclosure provides a class of multivalent compounds that is capable of specifically binding to a target protein at two or more distinct binding sites on the target protein. The term “multivalent” refers to interactions between a compound and a target protein that can occur at two or more separate and distinct sites of a target protein molecule. The multivalent D-peptidic compounds are capable of multiple binding interactions that can occur cooperatively to provide for high affinity binders to target proteins and potent biological effects on the function of the target protein. The term “multimeric” refers to a compound that includes two (i.e., dimeric), three (i.e., trimeric) or more monomeric peptidic units (e.g., domains). When the multimeric compound is homologous each peptidic unit can have the same binding property, i.e. each monomeric unit is capable of binding to the same binding site(s) on a target protein molecule. Such multimeric compounds can find use in binding target proteins that occur naturally as homodimers or are capable of multimerization. A dimeric compound can bind simultaneously to the two identical binding sites on the two molecules of the target protein homodimer. In some instances, depending on the target protein, the multivalent D-peptidic compounds of this disclosure can be multimerized, e.g., a dimeric bivalent D-peptidic compound can include a dimer of two bivalent D-peptidic compounds. In certain cases, the multimeric compound is heterologous and each peptidic unit (e.g., domain or bivalent unit) specifically binds a different target site or protein.

In some embodiments, the multivalent D-peptidic compound is homodimeric. In some embodiments, multivalent D-peptidic compounds include a first D-peptidic GA domain; and a second D-peptidic GA domain that is homologous to the first D-peptidic GA domain.

In some embodiments, the multivalent D-peptidic compound is homodimeric. In some embodiments, the multivalent D-peptidic compounds include a first D-peptidic Z domain, and a second D-peptidic Z domain that is homologous to the first D-peptidic Z domain.

The multivalent D-peptidic compound includes at least two D-peptidic domains where each domain has a specificity determining motif composed of variant amino acids configured to provide a interface of specific protein-protein interactions at a binding site. When multiple D-peptidic domains are linked together they can simultaneously contact the target protein and provide multiple interfaces at multiple binding sites. The multiple protein-protein binding interactions can occur cooperatively via an avidity effect to provide for significantly higher effective affinities than is possible to achieve for any one D-peptidic domain alone. The present disclosure discloses use of mirror image phage display screening using scaffolded small protein domain libraries to produce multiple D-peptidic domains binding multiple target binding sites, and that such domains can be successfully linked to produce high affinity binders exhibiting a strong avidity effect. The multimeric compounds demonstrated by the inventors have affinity comparable to or better than corresponding antibody agents and provide for effective biological activity against target proteins in vivo.

In general, the target protein is a naturally occurring L-protein and the compound is a D-peptidic compound. It is understood that for any of the D-peptidic compounds described herein, a L-peptidic version of the compound is also included in the present disclosure, which specifically binds to a D-target protein. The subject D-peptidic compounds were identified in part by using methods of mirror image screening of a variety of scaffolded domain phage display libraries for binding to a synthetic D-target protein. Any convenient proteins can be targets for the multivalent D-peptidic compounds of this disclosure. The target protein can be one that is associated with a disease or condition in a subject. Target proteins of interest include, but are not limited to, VEGF (e.g., VEGF-A, VEGF-B, VEGF-C, VEGF-D), Programmed cell death protein 1 (PD1), Programmed death-ligand 1 (PD-L1), Platelet-derived growth factor (PDGF) (e.g., PDGF-B), Tumor necrosis factor alpha (TNF-alpha), cytotoxic T-lymphocyte-associated protein 4 (CTLA-4), OX-40, Human Epidermal Growth Factor Receptor 2 (Her2), FcRn, Lymphocyte-Activation Gene (LAG) e.g., LAG-3, transferrin, CD3 ((cluster of differentiation 3 protein), calcitonin gene-related peptide (CGRP) and B-cell maturation antigen (BCMA).

The experimental section of the present disclosure describes in detail the results of studies directed to identifying and assessing D-peptidic GA domain and/or Z domain binders to PD-1 and VEGF-A. In addition, U.S. Provisional Application No. 62/865,469, filed Jun. 24, 2019, describes the results of a study to identify and assess D-peptidic GA domain compounds that specifically bind to VEGF-A, the disclosure of which is herein incorporated by reference. U.S. Provisional Application No. 62/822,241, filed Mar. 22, 2019, describes the results of studies to identify and assess bivalent D-peptidic compounds including GA and Z domains that specifically bind to VEGF-A with high affinity. In addition, the inventors have also identified D-peptidic GA domain binders to the following targets: Her2, BCMA and CD3 using the mirror image phage display methods described herein. The compounds were assessed using SPR and ELISA assays and shown to specifically bind their respective targets. In addition, the inventors have also identified D-peptidic Z domain binders to the following targets: Her2, BCMA and CD3 using the mirror image phage display methods described herein. The compounds were assessed using SPR, ELISA assays, and x-ray crystallography, and shown to specifically bind their respective targets. These results indicate the applicability of the subject multivalent D-peptidic compounds to a variety of target proteins of interest. In some embodiments, the subject multivalent D-peptidic compounds include linked D-peptidic GA and Z domains

D-peptidic compounds can provide a number of desirable properties for therapeutic applications in comparison to a corresponding L-polypeptide, such as proteolytic stability, substantially reduced immunogenicity and long in vivo half life. The D-peptidic compounds of this disclosure are generally significantly smaller in size by comparison to an antibody agent for a target protein. In some embodiments, the smaller size and properties of the subject compounds provide for routes of administration, tissue distribution and tissue penetration, and dosage regimens that are superior to antibody-based therapeutics.

This disclosure provides a multivalent D-peptidic compound including at least first and second D-peptidic domains. The first and second D-peptidic domains can specifically bind to distinct non-overlapping binding sites of the target protein and can be linked to each other via a linking component (e.g., as described herein). The linking component can be configured to allow for simultaneous or sequential binding to the target protein. By “sequential binding” it is meant that binding of the first D-peptidic domain to the target can increases the likelihood binding by the second D-peptidic domain will occur, even if binding does not occur simultaneously.

The first and second D-peptidic domains can be heterologous to each other, i.e., the domains are of different domain types. For example, the first D-peptidic domain may be a variant GA domain and the second D-peptidic domain may be a variant Z domain, or vice versa. In some embodiments, mirror image phage display screening of a target protein using two different scaffolded domain libraries provides variant domain binders that are directed towards two different binding sites on the target protein.

When the multivalent D-peptidic compound includes only two such domains it can be termed bivalent. In some embodiments, the D-peptidic compound is bivalent. Trivalent, tetravalent and higher multivalencies are also possible. In some embodiments, a D-peptidic compound further includes a third D-peptidic domain that specifically binds a target protein (e.g., trivalent, tetravalent, etc.). Trivalent D-peptidic compounds can include three D-peptidic domains connected via two linking components in a linear fashion, or via a single trivalent linking component. Trivalent D-peptidic compounds can include two of the same D-peptidic compounds connected via a disulfide linkage between two cysteine residues on each D-peptidic compound and a linking component between one of the disulfide linked D-peptidic compounds and a third D-peptidic compound. Tetravalent and higher multivalent compounds can similarly be linked in, either in a linear fashion via bivalent linking components, or in a branched configuration via one or more multivalent or branched linking components.

In some embodiments, a multivalent D-peptidic compound includes a first D-peptidic domain including a first three-helix bundle domain capable of specifically binding a first binding site of the target protein. In some embodiments, a multivalent D-peptidic compound includes a second D-peptidic domain including a second three-helix bundle domain capable of specifically binding a second binding site of the target protein.

In some embodiments, the first and second D-peptidic domains are selected from D-peptidic GA domain and D-peptidic Z domain. In some embodiments, the first D-peptidic domain is a D-peptidic GA domain; and the second D-peptidic domain is a D-peptidic Z domain.

### Linking Components

The term “linking component” is meant to cover multivalent moieties capable of establishing covalent links between two or more D-peptidic domains of the subject compounds. Sometimes, the linking component is bivalent. Alternatively, the linking component is trivalent or dendritic. A linking component may be installed during synthesis of D-peptidic domain polypeptides, or post-synthesis, e.g., via conjugation of two or more D-peptidic domains that are already folded. A linking component may be installed in a subject compound via conjugation of two D-peptidic domains using a bifunctional linker. A linking component may also be designed such that it may be incorporated during synthesis of the D-peptidic domain polypeptides, e.g., where the linking component is itself peptidic and is prepared via solid phase peptide synthesis (SPPS) of a sequence of amino acid residues. In addition, chemoselective functional groups and/or linkers may be installed during polypeptide synthesis to provide for facile conjugation of a D-peptidic domain after SPPS.

Any convenient linking groups or linkers can be adapted for use as a linking component in the subject multivalent compounds. Linking groups and linker units of interest include, but are not limited to, amino acid residue(s), PEG units, terminal-modified PEG (e.g., —NH(CH₂)ₘO[(CH₂)₂O]ₙ(CH₂)ₚCO— linking groups where m is 2-6, p is 1-6 and n is 1-50, such as 1-12 or 1-6), C2-C12alkyl or substituted C2-C12alkyl linkers, succinyl (e.g., —COCH₂CH₂CO—) units, diaminoethylene units (e.g., —NRCH₂CH₂NR— wherein R is H, alkyl or substituted alkyl) and combinations thereof, e.g., connected via linking functional groups such as amide, sulfonamide, carbamate, ether, thioether, ester, thioester, amino (—NH—) and the like. The linking component can be peptidic, e.g., a linker including a sequence of amino acid residues. The linking component can be a linker of formula -(L¹)ₐ-(L²)_{b}-(L′)_{c}-(L⁴)ₐ-(L′)ₑ-, where L¹ to L⁵ are each independently a linker unit, and a, b, c, d and e are each independently 0 or 1, wherein the sum of a, b, c, d and e is 1 to 5. Other linkers are also possible, as shown in the multimeric compounds described herein.

In some embodiments, the linking component is a linker connecting a terminal amino acid residue of the first D-peptidic domain to a terminal amino acid residue of the second D-peptidic domain (e.g., N-terminal to N-terminal linker or C-terminal to C-terminal linker). In some embodiments, the linking component is a linker connecting an amino acid sidechain of the first D-peptidic domain to a terminal amino acid residue of the second D-peptidic domain that are in proximity to each other when the first and second D-peptidic domains are simultaneously bound to the target protein. In some embodiments, the linking component is a linker connecting an amino acid sidechain of the first D-peptidic domain to a proximaln amino acid sidechain of the second D-peptidic domain that is proximal to the amino acid sidechain when the first and second D-peptidic domains are simultaneously bound to the target protein.

In some embodiments, the linking component includes one or more groups selected from amino acid residue, polypeptide, (PEG)ₙ linker (e.g., n is 2-50, 3-50, 4-50, 6-50 or 6-20), modified PEG moiety, C₍₁₋₆₎alkyl linker, substituted C₍₁₋₆₎alkyl linker, —CO(CH₂)ₘCO—, —NR(CH₂)ₚNR—, —CO(CH₂)ₘNR—, —CO(CH₂)ₘO—, —CO(CH₂)ₘS—, and linked chemoselective functional groups (e.g., —CONH—, —OCONH—, click chemistry conjugate such as 1,2,3-triazole, maleimide-thiol conjugate thiosuccinimide, haloacetyl-thiol conjugate thioether, etc.), wherein m is 1 to 6, p is 2-6 and each R is independently H, C₍₁₋₆₎alkyl or substituted C₍₁₋₆₎alkyl.

The linking component can include a terminal-modified PEG linker that is connected to the D-peptidic compounds using any convenient linking chemistry. PEG is polyethylene glycol. The term “terminal-modified PEG” refers to polyethylene glycol of any convenient length where one or both of the terminals are modified to include a chemoselective functional group suitable for conjugation, e.g., to another linking group moiety or to the terminal or sidechain of a peptidic compound. The Examples section describes use of several exemplary terminal-modified PEG bifunctional linkers having terminal maleimide functional groups for conjugating chemoselectively to a thiol group, such as a cysteine residue installed in the sequence of a D-peptidic domain. The D-peptidic compounds can be modified at the N- and/or C-terminals of the GA domain motifs to include one or more additional amino acid residues that can provide for a particular linkage or linking chemistry to connect to the Y group, such as a cysteine or a lysine.

Chemoselective reactive functional groups that may be utilized in linking the subject D-peptidic compounds via a linking group, include, but are not limited to: an amino group (e.g., a N-terminal amino or a lysine sidechain group), an azido group, an alkynyl group, a phosphine group, a thiol (e.g., a cysteine residue), a C-terminal thioester, aryl azides, maleimides, carbodiimides, N-hydroxysuccinimide (NHS)-esters, hydrazides, PFP-esters, hydroxymethyl phosphines, psoralens, imidoesters, pyridyl disulfides, isocyanates, aminooxy-, aldehyde, keto, chloroacetyl, bromoacetyl, and vinyl sulfones.

Any convenient multivalent linker may be utilized in the subject multimers. By multivalent is meant that the linker includes two or more terminal or sidechain groups suitable for attachment to components of the subject compounds, e.g., D-peptidic domains, as described herein. In some embodiments, the multivalent linker is bivalent or trivalent. In some instances, the multivalent linker Y is a dendrimer scaffold. Any convenient dendrimer scaffold may be adapted for use in the subject multimers. The dendrimer scaffold is a branched molecule that includes at least one branching point and two or more terminals suitable for connecting to the N-terminal or C-terminal of a domain via optional linkers. The dendrimer scaffold may be selected to provide a desired spatial arrangement of two or more domains. In some embodiments, the spatial arrangement of the two or more domains is selected to provide for a desired binding affinity and avidity for the target protein.

In some embodiments, the multivalent linker group is derived from/includes a chemoselective reactive functional group that is capable of conjugating to a compatible function group on a second D-peptidic domain. In certain cases, the multivalent linker group is a specific binding moiety (e.g., biotin or a peptide tag) that is capable of specifically binding to a multivalent binding moiety (e.g., a streptavidin or an antibody). In certain cases, the multivalent linker group is a specific binding moiety that is capable of forming a homodimer or a heterodimer directly with a second specific binding moiety of a second compound. As such, In some embodiments, where the compound includes a molecule of interest that includes a multivalent linker group, the compound may be part of a multimer. Alternatively, the compound may be a monomer that is capable of being multimerized either directly with one or more other compounds, or indirectly via binding to a multivalent binding moiety.

Linking Components that Link GA Domain and Z Domain

In some embodiments, a multivalent D-peptidic compound that specifically binds PD-1 includes a D-peptidic GA domain capable of specifically binding a first binding site of PD-1; and a D-peptidic Z domain capable of specifically binding a second binding site of PD-1.

In some embodiments, the linking component covalently links the D-peptidic GA and Z domains. In some embodiments, the linking component is configured to link the D-peptidic GA and Z domains whereby the domains are capable of simultaneously binding to PD1. In some embodiments, the linking component is configured to connect the D-peptidic GA and Z domains via sidechain and/or terminal groups that are proximal to each other when the D-peptidic GA and Z domains are simultaneously bound to PD1.

In some embodiments, the linking component includes a linker connecting a terminal of the D-peptidic GA domain to a terminal of the D-peptidic Z domain. In some embodiments, the linker connects the N-terminal residue of the D-peptidic GA domain polypeptide to the N-terminal residue of the D-peptidic Z domain polypeptide.

In some embodiments, the linking component connects a first amino acid sidechain of a residue of the D-peptidic GA domain and a second amino acid sidechain of a residue of the D-peptidic Z domain. In some embodiments, the linking component includes one or more groups selected from amino acid residue, polypeptide, (PEG)ₙ linker (e.g., n is 2-50, 3-50, 4-50, 6-50 or 6-20), modified PEG moiety, C₍₁₋₆₎alkyl linker, substituted C₍₁₋₆₎alkyl linker, —CO(CH₂)ₘCO—, —NR(CH₂)ₚNR—, —CO(CH₂)ₘNR—, —CO(CH₂)ₘO—, —CO(CH₂)ₘS—, and linked chemoselective functional groups (e.g., —CONH—, —OCONH—, click chemistry conjugate such as 1,2,3-triazole, maleimide-thiol conjugate thiosuccinimide, haloacetyl-thiol conjugate thioether, etc.), wherein m is 1 to 6, p is 2-6 and each R is independently H, C₁>6)alkyl or substituted C₍₁₋₆₎alkyl.

In some embodiments, the D-peptidic GA domain and the D-peptidic Z domain are conjugated to each other via N-terminal cysteine residues with a bis-maleimide linker or bis-haloacetyl linker, optionally including a (PEG)n moiety (e.g., n is 2-12, such as 3-8, e.g., a PEG3, PEG6, or PEG8 containing linker). It is understood that one or more additional linking units, e.g., as described above, can also be incorporated. In some cases, one or more additional spacer residues are incorporated between the terminal cysteine residues and the consensus domain sequence, e.g., a, G and/or s residues. In certain cases, a ca-dipeptide residue is added to the N-terminal of the domains before maleimide or haloacetyl-bifunctional linker conjugation.

In some embodiments, the linking component connecting the D-peptidic GA and Z domains is selected from:

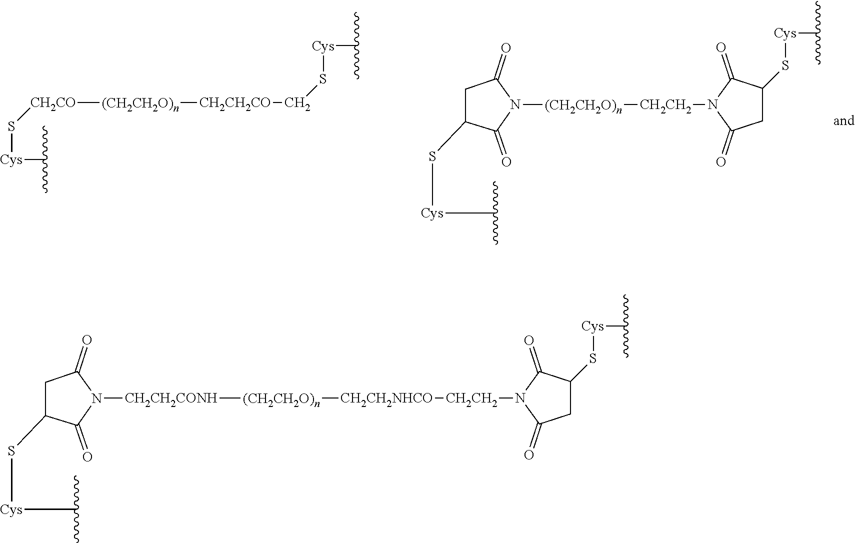

wherein n is 1-20 (e.g., 2 to 12, 2 to 8, or 3 to 6).

### Peptidic Domains

Any convenient peptidic domains can be utilized in the subject compounds. A variety of small protein domains are utilized in phage display screening that can be adapted for use in methods of mirror image screening against target proteins as described herein. A small peptidic domain of interest can consist of a single chain polypeptide sequence of 25 to 80 amino acid residues, such as 30 to 70 residues, 40 to 70 residues, 40 to 60 residues, 45 to 60 residues, 50 to 60 residues, or 52 to 58 residues. The peptidic domain can have a molecular weight (MW) of 1 to 20 kilodaltons (kDa), such as 2 to 15 kDa, 2 to 10 kDa, 2 to 8 kDa, 3 to 8 kDa or 4 to 6 kDa. In some embodiments, a D-peptidic domain consists essentially of a single chain polypeptide sequence of 30 to 80 residues (e.g., 40 to 70, 45 to 60 residues, 50 to 60 residues, or 52 to 58 residues), and has a MW of 1 to 10 kDa (e.g., 2 to 8 kDa, 3 to 8 kDa or 4 to 6 kDa).

The peptidic domain can be a three helix bundle domain. A three helix bundle domain has a structure consisting of two parallel helices and one anti-parallel helix joined by loop regions. Three helix bundle domains of interest include, but are not limited to, GA domains, Z domains and albumin-binding domains (ABD) domains.

Based on the present disclosure, it is understood that several of the amino acid residues of the D-peptidic domain motif which are not located at the target binding surface of the structure can be modified without having a significant detrimental effect on three dimensional structure or the target binding activity of the resulting modified compound. As such, several amino acids modifications/mutations can be incorporated into the subject compounds as needed in order to impart a desirable property on the compound, including but not limited to, increased water solubility, ease of chemical synthesis, cost of synthesis, conjugation site, stability, isoelectric point (pI), aggregation resistance and/or reduced non-specific binding. The positions of the mutations may be selected so as to avoid or minimize any disruption to the specificity determining motif (SDM) or the underlying three dimensional structure of the target binding domain motif that provides for specific binding to the target protein. For example, mutation of solvent exposed positions on the opposite side of the domain structure from the binding surface can be made to introduce desirable variant amino acid residues, e.g., to increase solubility or provide a desirable protein pI. In some embodiments, based on the three dimensional structure of the target binding domain motif, the positions of mutations can be selected to provide for increased stability (e.g., via introduction of variant amino acid(s) into the core packing residues of the structure) or increased binding affinity (e.g., via introduction of variant amino acid(s) in the SDM). In some instances, the compound includes two or more, such as 3 or more, 4 or more, 5 or more, 6 or more, 7 or more, 8 or more, 9 or more, or 10 or more surface mutations at positions that are not part of the binding surface to the target protein.

### Variant GA Domain

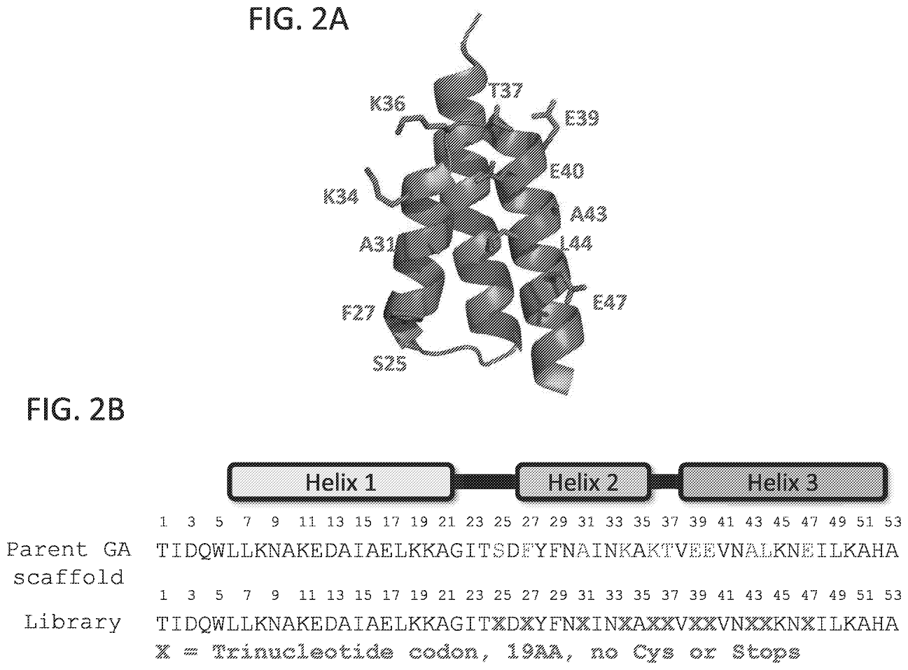

The term “GA domain” refers to a D-peptidic domain having a three-helix bundle tertiary structure that is related to the albumin binding domain of protein G. In the Protein Data Bank (PDB) structure 1tf0 provides an exemplary GA domain structure. FIG. 2A and FIG. 2B include depictions of a native GA domain structure and one exemplary sequence of an unmodified native GA domain. The term “GA domain scaffold” refers to an underlying GA domain sequence which provides a characteristic 3-helix bundle structure and can be adapted for use in the subject compounds. In some embodiments the GA domain scaffold has a consensus sequence defined in Table 1. Table 1 provides a list of exemplary GA domain scaffold sequences which can be adapted for use in the subject compounds. A “variant GA domain” is a GA domain that includes variant amino acids at select positions of the three-helix bundle tertiary structure which together provide for specific binding to a target protein.

A GA domain can be described by the formula:

[Helix 1]-[Linker 1]-[Helix 2]-[Linker 2]-[Helix 3]

where [Helix 1], [Helix 2] and [Helix 3] are helical regions of a characteristic three-helix bundle linked via D-peptidic linkers [Linker 1] and [Linker 2]. In the three-helix bundle, [Helix 1], [Helix 2] and [Helix 3] are linked D-peptidic regions wherein [Helix 2] is configured substantially anti-parallel to a two-helix complex of parallel alpha helices [Helix 1] and [Helix 3]. [Linker 1] and [Linker 3] can each independently include a sequence of 1 to 10 amino acid residues. In some embodiments, [Linker 1] is longer in length than [Linker 3]. The GA domain can be a D-peptidic sequence of between 30 and 90 residues, such as between 30 and 80 residues, between 40 and 70 residues, between 45 and 60 residues, between 45 and 60 residues, or between 45 and 55 residues. In certain instances, the GA domain motif is a D-peptidic sequence of between 35 and 55 residues, such as between 40 and 55 residues, or between 45 and 55 residues. In certain embodiments, the GA domain motif is a D-peptidic sequence of 45, 46, 47, 48, 49, 50, 51, 52 or 53 residues.

GA domains of interest include those described by Jonsson et al. (Engineering of a femtomolar affinity binding protein to human serum albumin, Protein Engineering, Design & Selection, 21(8), 2008, 515-527), the disclosure of which is herein incorporated by reference in its entirety, and which includes a GA domain and phage display library having a scaffold sequence (G148-GA3) with library mutations at positions 25, 27, 31, 34, 36, 37, 39, 40, 43, 44 and 47 of the scaffold. Other GA domains of interest include but are not limited to those described in U.S. Pat. Nos. 6,534,628 and 6,740,734, the disclosures of which are herein incorporated by reference in their entirety.

The variant GA domains of this disclosure can have a specificity-determining motif (SDM) that includes 5 or more variant amino acid residues at positions selected from 25, 27, 30, 31, 34, 36, 37, 39, 40 and 42-48. In some instances, the specificity-determining motif (SDM) further includes a variant amino acid at position 28 of a GA domain.

### Locked Variant GA Domain

This disclosure includes variant GA domain compounds having an interhelix linker or bridge between adjacent residues of helix 1 and helix 3. The term “locked variant GA domain” refers to a variant GA domain that includes a structure stabilizing linker between any two helices of GA domain. Sometimes, the linked adjacent residues are located at the ends of the helices 1 and 3. FIG. 2A shows a ribbon structure of a GA scaffold domain that illustrates the configuration of helices 1-3 in the three-helix bundle. The interhelix linker can be located between amino acid residues at positions 7 (helix 1) and 38 (helix 3) of the domain which are proximate to each other in the three dimensional structure of the domain. Positions 7 and 38 can be considered to be core facing residues located at the ends of helices that are capable of making stabilizing contacts with the hydrophobic core of the structure. The interhelix linker can have a backbone of 3 to 7 atoms in length as measured between the alpha-carbons of the linked amino acid residues. For example a disulfide linkage between two cysteine residues provides a backbone of 4 atoms in length (—CH₂—S—S—CH₂—) between the alpha-carbons of the two cysteine amino acid residues.

A variety of compatible natural and non-naturally occurring amino acid residues can be incorporated at positions 7 and 38 of a GA domain and which are able to be conjugated to each other to provide for the interhelix linker. Compatible residues include, but are not limited to, aspartate or glutamate linked to serine or cysteine via ester or thioester linkage, aspartate or glutamate linked to ornithine or lysine via an amide linkage. As such, the interhelix linker can include one or more groups selected from C₍₁₋₆₎alkyl, substituted C₍₁₋₆₎alkyl, —(CHR)ₙ—CONH—(CHR)ₘ—, and —(CHR)ₙ—S—S—(CHR)ₘ—, wherein each R is independently H, C₍₁₋₆₎alkyl or substituted C₍₁₋₆₎alkyl and n+m=2, 3, 4 or 5. Any convenient non-naturally occurring residues can be utilized to incorporate compatible chemoselective tags at the amino acid residue sidechains of positions 7 and 38, e.g., click chemistry tags such as azide and alkyne tags, which can be conjugated to each other post polypeptide synthesis.

Incorporation of an intradomain linker can provide an increase in stability and/or binding affinity for target protein. In some embodiments, the binding affinity (K_{D}) of the D-peptidic compound for target protein (e.g., PD-1) is 3-fold or more stronger (i.e., a 3-fold lower K_{D}) than a control polypeptide lacking the intradomain linker, such as 5-fold or more stronger, 10-fold or more stronger, 30 fold or more stronger, or even stronger. It is understood that the features of a locked variant GA domain (e.g., as described herein) can be adapated for use in compounds which bind to any convenient target protein. Exemplary locked variant GA domain compounds that specifically bind PD-1 are described below in greater detail.

In some embodiments, a variant GA domain polypeptide can include a N-terminal region from position 1 to about position 6 that can be considered non-overlapping with Helix 2 and Helix 3 because this region is not directly involved in contacts with the adjacent helix 2-loop-helix 3 region of the folded three helix bundle structure. In some embodiments, in the subject D-peptidic compounds, a N-terminal region from positions 1-5 of the GA domain can be optionally retained in the sequence and optimized to provide for a desirable property, such as increased water solubility, stability or affinity. It is understood that the N-terminal region of the variant D-peptidic compounds can be substituted, modified or truncated without significantly adversely affecting the activity of the compound. The N-terminal region can be modified to provide for conjugation or linkage to a molecule of interest (e.g., as described herein), or to another D-peptidic domain or multivalent compound (e.g., as described herein). In some embodiments, the N-terminal residues have a helical propensity that provides for an extended helical structure of Helix 1. Alternatively, the N-terminal region can incorporate helix capping residues that stabilize the N-terminus of helix 1.

### PD-1 Specific Variant GA Domain

This disclosure provides D-peptidic variant GA domain polypeptides that specifically bind PD-1. The polypeoptides can include a specificity-determining motif (SDM) defined by 5 or more variant amino acid residues (e.g., 5, 6, 7, 8, 9, 10 or 11 variant amino acid residues) at positions selected from 25, 27, 31, 34, 36, 37, 39, 40, 43, 44 and 47. It is understood that a variety of underlying GA domain scaffolds can be utilized to provide the characteristic three dimensional structure. For purposes of describing some exemplary PD-1 specific variant GA domain polypeptides of this disclosure, a numbered 53 residue scaffold sequence of FIG. 2B is utilized.

Exemplary PD-1 binding D-peptidic variant GA domain polypeptides include those of Table 2 and described by the sequences of compounds 977296-977299 (SEQ ID NOs: 32-35). In view of the structures and sequence variants described in the present disclosure, it is understood that a number of amino acid substitutions may be made to the sequences of the exemplary compounds while retaining specific binding to PD-1. By selecting positions of the variant GA domain where variability is tolerated without adversely affecting the three dimensional architecture of the GA domain, a number of amino acid substitutions may be incorporated.

Exemplary PD-1 binding D-peptidic variant GA domain polypeptides include those of Table 2 and described by the sequences of compounds 977978-977979 (SEQ ID NOs: 21-22). In view of the structures and sequence variants described in the present disclosure, it is understood that a number of amino acid substitutions may be made to the sequences of the exemplary compounds while retaining specific binding to PD-1. By selecting positions of the variant GA domain where variability is tolerated without adversely affecting the three dimensional architecture of the GA domain, a number of amino acid substitutions may be incorporated.

As such, this disclosure includes a sequence of one of compounds 977296 to 977299 (SEQ ID NOs: 32-35) having 1-10 amino acid substitutions (e.g., 1-8, 1-6 or 1-5, such as 1, 2, 3, 4 or 5 substitutions). The 1-10 amino acid substitutions can be substitutions for amino acids based on physical properties of the amino acid sidechains, e.g., according to Table 5. Sometimes, an amino acid of a sequence of 977296 to 977299 (SEQ ID NOs: 32-35) is substituted with a similar amino acid according to Table 5. In some embodiments, the substitution is for a conservative amino acid substitution or a highly conservative amino acid substitution according to Table 5. This disclosure also includes a sequence of one of compounds 977978-977979 (SEQ ID NOs: 21-22) having 1-10 amino acid substitutions (e.g., 1-8, 1-6 or 1-5, such as 1, 2, 3, 4 or 5 substitutions). The 1-10 amino acid substitutions can be substitutions for amino acids based on physical properties of the amino acid sidechains, e.g., according to Table 5. Sometimes, an amino acid of a sequence of 977978-977979 (SEQ ID NOs: 21-22) is substituted with a similar amino acid according to Table 5. In some embodiments, the substitution is for a conservative amino acid substitution or a highly conservative amino acid substitution according to Table 5.

This disclosure includes PD-1 binding D-peptidic variant GA domain polypeptides described by a sequence having 80% or more sequence identity with a sequence of 977296 to 977299 (SEQ ID NOs: 32-35), such as 85% or more, 87% or more, 89% or more, 91% or more, 93% or more, 94% or more, 96% or more, 98% or more sequence identity. In some embodiments, the variant GA domain polypeptide includes a sequence having 80% or more sequence identity with a sequence of 977296 (SEQ ID NO: 32), such as 85% or more, 87% or more, 89% or more, 91% or more, 93% or more, 94% or more, 96% or more, 98% or more sequence identity. In some embodiments, the variant GA domain polypeptide includes a sequence having 80% or more sequence identity with a sequence of 977297 (SEQ ID NO: 33), such as 85% or more, 87% or more, 89% or more, 91% or more, 93% or more, 94% or more, 96% or more, 98% or more sequence identity. In some embodiments, the variant GA domain polypeptide includes a sequence having 80% or more sequence identity with a sequence of 977298 (SEQ ID NO: 34), such as 85% or more, 87% or more, 89% or more, 91% or more, 93% or more, 94% or more, 96% or more, 98% or more sequence identity. In some embodiments, the variant GA domain polypeptide includes a sequence having 80% or more sequence identity with a sequence of 977299 (SEQ ID NO: 35), such as 85% or more, 87% or more, 89% or more, 91% or more, 93% or more, 94% or more, 96% or more, 98% or more sequence identity.

This disclosure includes PD-1 binding D-peptidic variant GA domain polypeptides described by a sequence having 80% or more sequence identity with a sequence of 977978-977979 (SEQ ID NOs: 21-22), such as 85% or more, 87% or more, 89% or more, 91% or more, 93% or more, 94% or more, 96% or more, 98% or more sequence identity. In some embodiments, the variant GA domain polypeptide includes a sequence having 80% or more sequence identity with a sequence of 977978 (SEQ ID NO: 21), such as 85% or more, 87% or more, 89% or more, 91% or more, 93% or more, 94% or more, 96% or more, 98% or more sequence identity. In some embodiments, the variant GA domain polypeptide includes a sequence having 80% or more sequence identity with a sequence of 977979 (SEQ ID NO: 22), such as 85% or more, 87% or more, 89% or more, 91% or more, 93% or more, 94% or more, 96% or more, 98% or more sequence identity.

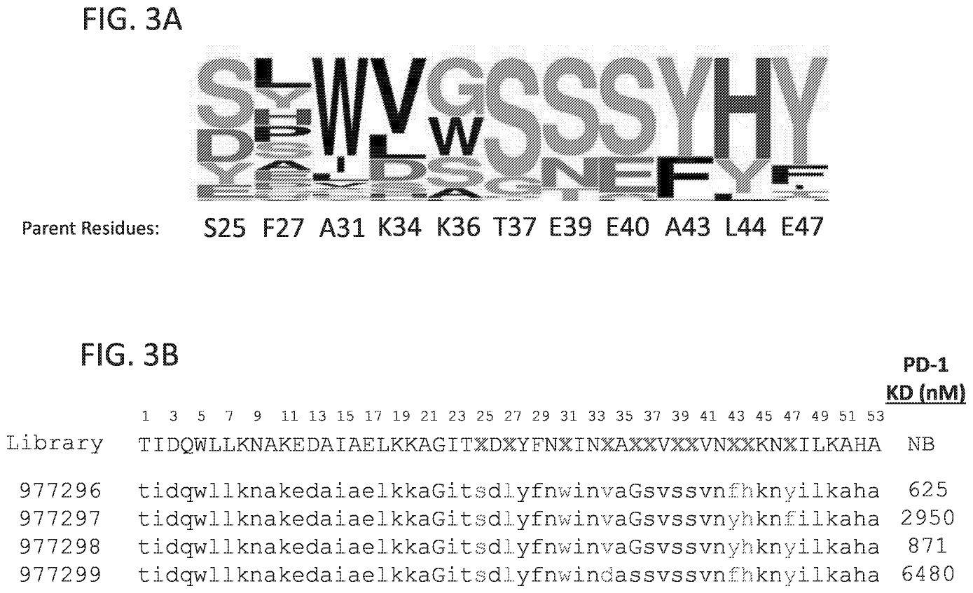

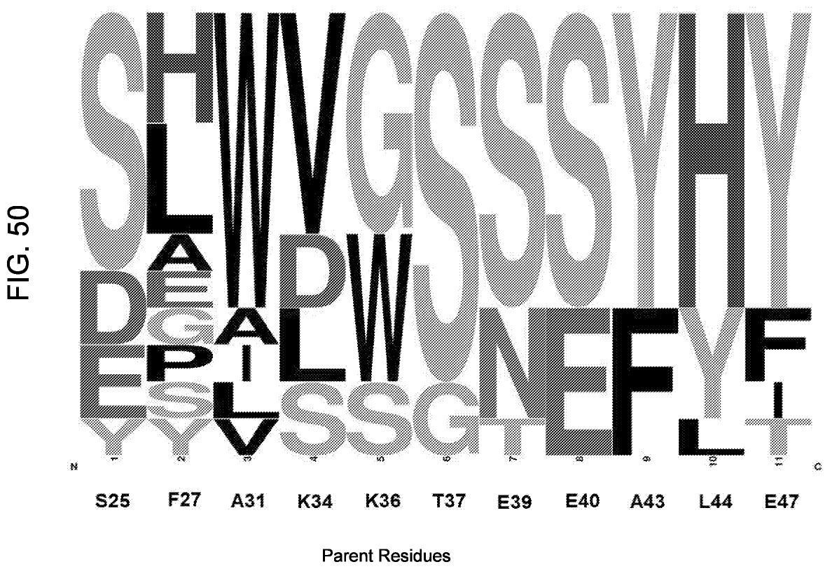

The PD-1 binding D-peptidic variant GA domain polypeptides can have amino acid residues at positions 25, 27, 31, 34, 36, 37, 39, 40, 43, 44 and 47 are consistent with the specificity-determining motif (SDM) defined in FIG. 3A and FIG. 50. In some embodiments, the specificity-determining motif (SDM) is defined by the following sequence motif:

(SEQ ID NO: 67)
s²⁵-l²⁷---w³¹--x³⁴-x³⁶s³⁷-s³⁹s⁴⁰--x⁴³h⁴⁴--x⁴⁷

wherein x³⁴, x³⁶, x⁴³ and x⁴⁷ are each independently any amino acid residue. In certain cases of the SDM:

x³⁴ is selected from v and d;

x³⁶ is selected from G and s;

x⁴³ is selected from f and y; and

x⁴⁷ is selected from f and y.

In certain cases, the specificity-determining motif (SDM) is:

(SEQ ID NO: 69)
s²⁵-l²⁷---w³¹-v³⁴-G³⁶s³⁷-s³⁹s⁴⁰--f⁴³h⁴⁴--y⁴⁷.

In some embodiments, the disclosure provides a D-peptidic compound that specifically binds PD-1, including: a D-peptidic GA domain including: a) a PD-1 specificity-determining motif (SDM) defined by the following amino acid residues: s²⁵-I²⁷-w³¹-x³⁴-x³⁶s³⁷-s³⁹s⁴⁰-x⁴³h⁴⁴-x⁴⁷ (SEQ ID NO: 67) wherein:
x³⁴ is selected from v and d;
x³⁶ is selected from G and s;
x⁴³ is selected from f and y; and
x⁴⁷ is selected from f and y.

In some embodiments, the D-peptidic compound includes a PD-1 SDM defined as having 80% or more (e.g., 90% or more) identity with the SDM residues defined in (a) as shown above (e.g. s²⁵-I²⁷-w³¹-x³⁴-x³⁶s³⁷-s³⁹s⁴⁰-x⁴³h⁴⁴-x⁴⁷ (SEQ ID NO: 67)). In some embodiments, the PD-I SDM is defined as having 1 to 3 amino acid residue substitutions relative to the SDM residues defined in (a) as shown above (e.g. s²⁵-I²⁷-w³¹-x³⁴-x³⁶s³⁷-s³⁹s⁴⁰-x⁴³h⁴⁴-x⁴⁷ (SEQ ID NO: 67)), wherein the 1 to 3 amino acid residue substitutions are selected from: i) a similar amino acid residue substitution according to Table 1; ii) a conservative amino acid residue substitution according to Table 1; iii) a highly conserved amino acid residue substitution according to Table 1; and iv) an amino acid residue substitution according to the motif defined in FIG. 3A and FIG. 50.

In some embodiments, SDM residues defined in (a) as shown above (e.g. s²⁵-I²⁷-w³¹-x³⁴-x³⁶s³⁷-s³⁹s⁴⁰-x⁴³h⁴⁴-x⁴⁷ (SEQ ID NO: 67)) are:

(SEQ ID NO: 68)
s²⁵-l²⁷---w³¹-v³⁴-G³⁶s³⁷-s³⁹s⁴⁰--x⁴³h⁴⁴--y⁴⁷

wherein x⁴³ is selected from f and y.

In some embodiments, the PD-1 SDM is defined by the following residues:

s²⁵-l²⁷---w³¹--v³⁴-G³⁶s³⁷-s³⁹s⁴⁰--f⁴³h⁴⁴--y⁴⁷
or
(SEQ ID NO: 70)
s²⁵-l²⁷---w³¹--v³⁴-G³⁶s³⁷-s³⁹s⁴⁰--y⁴³h⁴⁴--y⁴⁷

In some embodiments, the SDM residues are comprised in a polypeptide including: a) D-peptidic framework residues defined b the following amino acid residues:

(SEQ ID NO: 71)
-d²⁶-y²⁸fn-i³²n-a³⁵--v³⁸--v⁴¹n--k⁴⁵n-.

In some embodiments, the SDM residues are comprised in a polypeptide including b) D-peptidic framework residues having 80% or more (e.g., 90% or more) identity with the residues defined in (a) as shown above (-d²⁶-y²⁸fn-i³²n-a³⁵-v³⁸-v⁴¹n-k⁴⁵n- (SEQ ID NO: 71));

In some embodiments, the SDM residues are comprised in a polypeptide including c) D-peptidic framework residues having 1 to 3 amino acid residue substitutions relative to the residues defined in (a) as shown above (-d²⁶-y²⁸fn-i³²n-a³⁵-v³⁸-v⁴¹n-k⁴⁵n- (SEQ ID NO: 71)), wherein the 1 to 3 amino acid residue substitutions are selected from: i) a similar amino acid residue substitution according to Table 1; ii) a conservative amino acid residue substitution according to Table 1; and iii) a highly conserved amino acid residue substitution according to Table 1.

In some embodiments, the SDM-containing sequence includes 80% or more (e.g., 85% or more, 90% or more, or 95% or more) identity to the amino acid sequence:

(SEQ ID NO: 52)
s²⁵dlyfnwinx³⁴ax³⁶svssvnx⁴³hknx⁴⁷;

wherein:

x³⁴ is selected from v and d;

x³⁶ is selected from G and s;

x⁴³ is selected from f and y; and

x⁴⁷ is selected from f and y.

In some embodiments, a GA domain includes a three-helix bundle of the structural formula:

[Helix 1(#⁶-21>]-[Linker 1(#²²-26>]-[Helix 2^{(#27-35)}]-[Linker 2^{(#36-37)}]-[Helix 3^{(#38-51)}]

wherein: # denotes reference positions of amino acid residues comprised in the D-peptidic GA domain; and Helix 1^{(#6-21)} includes a D-peptidic framework sequence selected from: a) l⁶lknakedaiaelkka²¹ (SEQ ID NO: 53); b) a sequence having 70% or more (e.g., 75% or more, 80% or more, 85% or more, or 90% or more) identity to the amino acid sequence set forth in (a) (e.g. l⁶lknakedaiaelkka²¹ (SEQ ID NO: 53)); and c) a sequence having 1 to 5 amino acid residue substitutions relative to the sequence defined in (a) as shown above (l⁶lknakedaiaelkka²¹ (SEQ ID NO: 53)), wherein the 1 to 5 amino acid residue substitutions are selected from: i) a similar amino acid residue substitution according to Table 1; ii) a conservative amino acid residue substitution according to Table 1; and iii) a highly conserved amino acid residue substitution according to Table 1.

In some embodiments, GA domain includes one or more segments of a D-peptidic framework sequence selected from: a) N-terminal segment: t¹idgw⁵ (SEQ ID NO: 54); Loop 1 segment: G²²it²⁴ (SEQ ID NO: 55); and C-terminal segment: i⁴⁸lkaha⁵³ (SEQ ID NO: 56); or b) one or more segments having 60% or more sequence identity relative to the one or more segments defined in (a) (e.g. N-terminal segment: t¹idgw⁵ (SEQ ID NO: 54); Loop 1 segment: G²²it²⁴ (SEQ ID NO: 55); and C-terminal segment: i⁴⁸lkaha⁵³ (SEQ ID NO: 56)); or c) one or more segments each independently having 0 to 3 amino acid substitutions relative to the segments defined in (a) as shown above (e.g. N-terminal segment: t¹idgw⁵ (SEQ ID NO: 54); Loop 1 segment: G²²it²⁴ (SEQ ID NO: 55); and C-terminal segment: i⁴⁸lkaha⁵³ (SEQ ID NO: 56)), wherein the 0 to 3 amino acid substitutions are selected from: i) a similar amino acid residue substitution according to Table 1; ii) a conservative amino acid residue substitution according to Table 1; and iii) a highly conserved amino acid residue substitution according to Table 1.

In some embodiments, the D-peptidic GA domain includes: (a) a sequence selected from one of compounds 977296 to 977299 (SEQ ID NOs: 32-35); (b) a sequence having 80% or more identity with the sequence defined in (a) (e.g. 977296 to 977299 (SEQ ID NOs: 32-35)); or (c) a sequence having 1 to 10 (e.g., 1 to 6, 1 to 5, 1 to 4, 1 to 3, 1 to 2, 2 or 1) amino acid residue substitution(s) relative to the sequence defined in (a) (e.g. 977296 to 977299 (SEQ ID NOs: 32-35)), wherein the 1 to 10 amino acid substitutions are: i) a similar amino acid residue substitution according to Table 1; ii) a conservative amino acid residue substitution according to Table 1; or iii) a highly conserved amino acid residue substitution according to Table 1.

In some embodiments, the D-peptidic GA domain includes a polypeptide of one of compounds 977296 to 977299 (SEQ ID NOs: 32-35). In some embodiments, the D-peptidic GA domain includes a polypeptide of one of compounds 977978-977979 (SEQ ID NOs: 21-22).

### Variant Z Domain

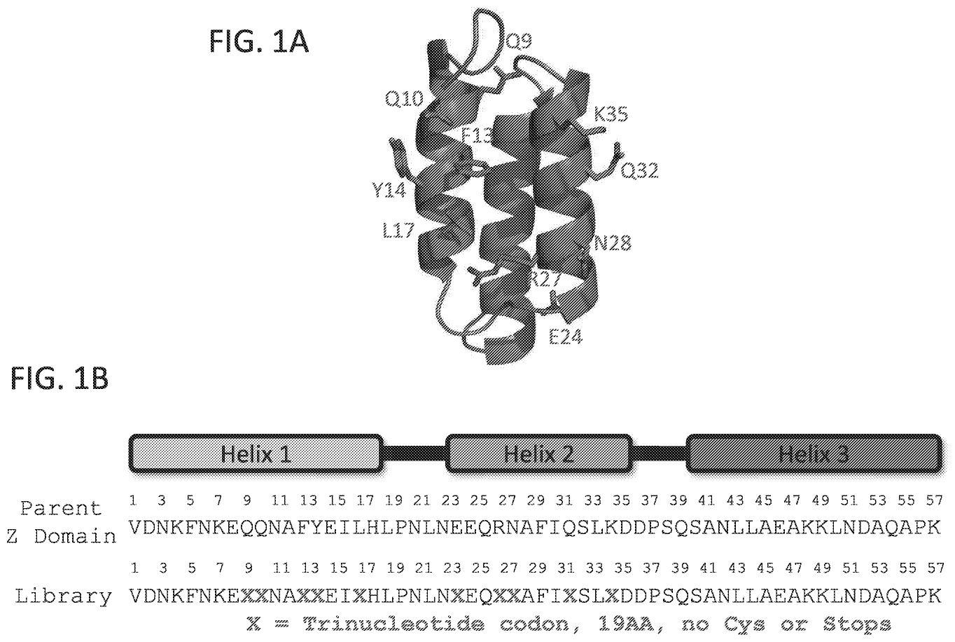

The term “Z domain” refers to a peptidic domain having a three-helix bundle tertiary structure that is related to the immunoglobulin G binding domain of protein A. In the Protein Data Bank (PDB), structure 2spz provides an exemplary Z domain structure. See also, FIG. 1A and FIG. 1B which include depictions of a native Z domain structure and one exemplary sequence of an unmodified native Z domain. The term “Z domain scaffold” refers to an underlying Z domain sequence which provides a characteristic 3-helix bundle structure and can be adapted for use in the subject compounds. In some embodiments, the Z domain scaffold has a consensus sequence defined by one of the sequences of Table 1. Table 1 also provides a list of exemplary Z domain scaffold sequences which can be adapted for use in the subject compounds. A “variant Z domain” is a Z domain including variant amino acids at select positions of the three-helix bundle tertiary structure that provide for specific binding to a target protein. A Z domain motif can be generally described by the formula:

[Helix 3]-[Linker 1]-[Helix 2]-[Linker 2]-[Helix 1]

wherein [Linker 1] and [Linker 2] are independently D-peptidic linking sequences of between 1 and 10 residues and [Helix 1], [Helix 2] and [Helix 3] are as described above for the GA domain.

Z domains of interest include, but are not limited to, those described by Nygren (“Alternative binding proteins: Affibody binding proteins developed from a small three-helix bundle scaffold”, FEBS Journal 275 (2008) 2668-2676), US20160200772, U.S. Pat. No. 9,469,670 and a 33-residue minimized Z domain of protein A described by Tjhung et al. (Front. Microbiol., 28 Apr. 2015), the disclosures of which are herein incorporated by reference in their entirety.

### PD-1 Specific Variant Z Domain

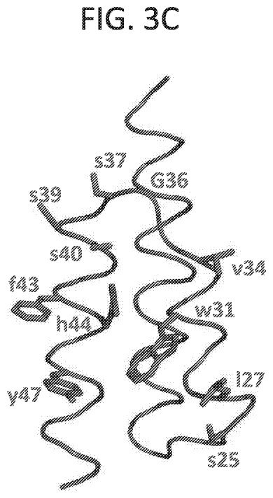

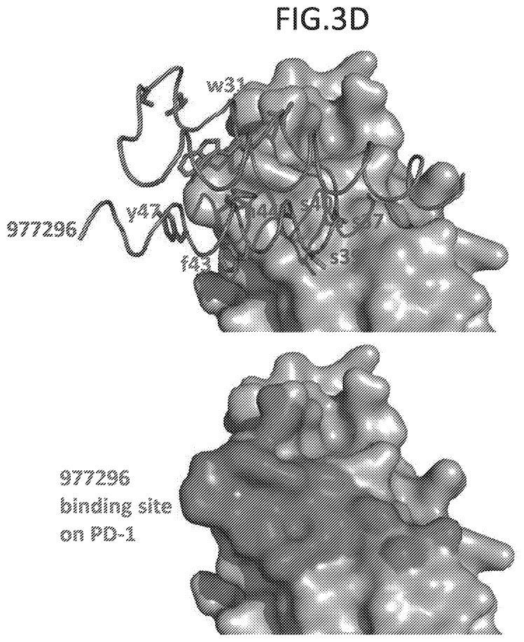

This disclosure provides D-peptidic variant Z domain polypeptides that specifically bind PD-1. The polypeoptides can include a specificity-determining motif (SDM) defined by 5 or more variant amino acid residues (e.g., 5, 6, 7, 8, 9 or 10 variant amino acid residues) located at positions 9, 10, 13, 14, 17, 24, 27, 28, 32 and/or 35 of a Z domain polypeptide. It is understood that a variety of underlying Z domain scaffolds can be utilized to provide the characteristic three dimensional structure. For purposes of describing some exemplary PD-1 specific variant Z domain polypeptides of this disclosure, a numbered 57 residue scaffold sequence of FIG. 4B is utilized.

Exemplary PD-1 binding D-peptidic variant Z domain polypeptides include those of Table 2 and described by the sequences of compounds 978060 to 978065, and 981195 to 981197 (SEQ ID NOs: 36-44). In view of the structures and sequence variants described in the present disclosure, it is understood that a number of amino acid substitutions may be made to the sequences of the exemplary compounds while retaining specific binding to PD-1. By selecting positions of the variant Z domain where variability is tolerated without adversely affecting the three dimensional architecture of the Z domain, a number of amino acid substitutions may be incorporated. Additional exemplary PD-1 binding D-peptidic variant Z domain polypeptides include those of Table 2 and described by the sequences of compounds 979259 to 979262 and 979264 to 979269 (SEQ ID NOs: 24-33). In view of the structures and sequence variants described in the present disclosure, it is understood that a number of amino acid substitutions may be made to the sequences of the exemplary compounds while retaining specific binding to PD-1. By selecting positions of the variant Z domain where variability is tolerated without adversely affecting the three dimensional architecture of the Z domain, a number of amino acid substitutions may be incorporated.

As such, this disclosure includes a sequence of 978060 to 978065 and 981195 to 981197 (SEQ ID NOs: 36-44) having 1-10 amino acid substitutions (e.g., 1-8, 1-6 or 1-5 substitutions, such as 1, 2, 3, 4 or 5 amino acid substitutions). The 1-10 amino acid substitutions can be substitutions for amino acids based on physical properties of the amino acid sidechains, e.g., according to Table 5. Sometimes, an amino acid of a sequence of 978060 to 978065 and 981195 to 981197 (SEQ ID NOs: 36-44) is substituted with a similar amino acid according to Table 5. In some embodiments, the substitution is for a conservative amino acid substitution or a highly conservative amino acid substitution according to Table 5. This disclosure also includes a sequence of 979259 to 979262 and 979264 to 979269 (SEQ ID NOs: 24-33) having 1-10 amino acid substitutions (e.g., 1-8, 1-6 or 1-5 substitutions, such as 1, 2, 3, 4 or 5 amino acid substitutions). The 1-10 amino acid substitutions can be substitutions for amino acids based on physical properties of the amino acid sidechains, e.g., according to Table 5. Sometimes, an amino acid of a sequence of 979259 to 979262 and 979264 to 979269 (SEQ ID NOs: 24-33) is substituted with a similar amino acid according to Table 5. In some embodiments, the substitution is for a conservative amino acid substitution or a highly conservative amino acid substitution according to Table 5.

This disclosure includes PD-1 binding D-peptidic variant Z domain polypeptides described by a sequence having 80% or more sequence identity with a sequence of 978060 to 978065 and 981195 to 981197 (SEQ ID NOs: 36-44), such as 85% or more, 87% or more, 89% or more, 91% or more, 93% or more, 94% or more, 96% or more, 98% or more sequence identity. This disclosure includes PD-1 binding D-peptidic variant Z domain polypeptides described by a sequence having 80% or more sequence identity with a sequence of 979259 to 979262 and 979264 to 979269 (SEQ ID NOs: 24-34). In some embodiments, the D-peptidic variant Z domain polypeptide includes a sequence having 80% or more sequence identity with a sequence of 981195 (SEQ ID NO: 36), such as 85% or more, 87% or more, 89% or more, 91% or more, 93% or more, 94% or more, 96% or more, 98% or more sequence identity, such as 85% or more, 87% or more, 89% or more, 91% or more, 93% or more, 94% or more, 96% or more, 98% or more sequence identity. In some embodiments, the D-peptidic variant Z domain polypeptide includes a sequence having 80% or more sequence identity with a sequence of 978060 (SEQ ID NO: 25), such as 85% or more, 87% or more, 89% or more, 91% or more, 93% or more, 94% or more, 96% or more, 98% or more sequence identity. In some embodiments, the D-peptidic variant Z domain polypeptide includes a sequence having 80% or more sequence identity with a sequence of 978061 (SEQ ID NO: 26), such as 85% or more, 87% or more, 89% or more, 91% or more, 93% or more, 94% or more, 96% or more, 98% or more sequence identity. In some embodiments, the D-peptidic variant Z domain polypeptide includes a sequence having 80% or more sequence identity with a sequence of 978062 (SEQ ID NO: 27), such as 85% or more, 87% or more, 89% or more, 91% or more, 93% or more, 94% or more, 96% or more, 98% or more sequence identity. In some embodiments, the D-peptidic variant Z domain polypeptide includes a sequence having 80% or more sequence identity with a sequence of 978064 (SEQ ID NO: 28), such as 85% or more, 87% or more, 89% or more, 91% or more, 93% or more, 94% or more, 96% or more, 98% or more sequence identity. In some embodiments, the D-peptidic variant Z domain polypeptide includes a sequence having 80% or more sequence identity with a sequence of 978065 (SEQ ID NO: 29), such as 85% or more, 87% or more, 89% or more, 91% or more, 93% or more, 94% or more, 96% or more, 98% or more sequence identity. In some embodiments, the D-peptidic variant Z domain polypeptide includes a sequence having 80% or more sequence identity with a sequence of 981195 (SEQ ID NO: 42), such as 85% or more, 87% or more, 89% or more, 91% or more, 93% or more, 94% or more, 96% or more, 98% or more sequence identity. In some embodiments, the D-peptidic variant Z domain polypeptide includes a sequence having 80% or more sequence identity with a sequence of 981196 (SEQ ID NO: 43), such as 85% or more, 87% or more, 89% or more, 91% or more, 93% or more, 94% or more, 96% or more, 98% or more sequence identity. In some embodiments, the D-peptidic variant Z domain polypeptide includes a sequence having 80% or more sequence identity with a sequence of 981197 (SEQ ID NO: 44), such as 85% or more, 87% or more, 89% or more, 91% or more, 93% or more, 94% or more, 96% or more, 98% or more sequence identity. In some embodiments, the D-peptidic variant Z domain polypeptide includes a sequence having 80% or more sequence identity with a sequence of 979259 (SEQ ID NO: 24), such as 85% or more, 87% or more, 89% or more, 91% or more, 93% or more, 94% or more, 96% or more, 98% or more sequence identity. In some embodiments, the D-peptidic variant Z domain polypeptide includes a sequence having 80% or more sequence identity with a sequence of 979260 (SEQ ID NO: 25), such as 85% or more, 87% or more, 89% or more, 91% or more, 93% or more, 94% or more, 96% or more, 98% or more sequence identity. In some embodiments, the D-peptidic variant Z domain polypeptide includes a sequence having 80% or more sequence identity with a sequence of 979261 (SEQ ID NO: 26), such as 85% or more, 87% or more, 89% or more, 91% or more, 93% or more, 94% or more, 96% or more, 98% or more sequence identity. In some embodiments, the D-peptidic variant Z domain polypeptide includes a sequence having 80% or more sequence identity with a sequence of 979262 (SEQ ID NO: 27), such as 85% or more, 87% or more, 89% or more, 91% or more, 93% or more, 94% or more, 96% or more, 98% or more sequence identity. In some embodiments, the D-peptidic variant Z domain polypeptide includes a sequence having 80% or more sequence identity with a sequence of 979264 (SEQ ID NO: 28), such as 85% or more, 87% or more, 89% or more, 91% or more, 93% or more, 94% or more, 96% or more, 98% or more sequence identity. In some embodiments, the D-peptidic variant Z domain polypeptide includes a sequence having 80% or more sequence identity with a sequence of 979265 (SEQ ID NO: 29), such as 85% or more, 87% or more, 89% or more, 91% or more, 93% or more, 94% or more, 96% or more, 98% or more sequence identity. In some embodiments, the D-peptidic variant Z domain polypeptide includes a sequence having 80% or more sequence identity with a sequence of 979266 (SEQ ID NO: 30), such as 85% or more, 87% or more, 89% or more, 91% or more, 93% or more, 94% or more, 96% or more, 98% or more sequence identity. In some embodiments, the D-peptidic variant Z domain polypeptide includes a sequence having 80% or more sequence identity with a sequence of 979267 (SEQ ID NO: 31), such as 85% or more, 87% or more, 89% or more, 91% or more, 93% or more, 94% or more, 96% or more, 98% or more sequence identity. In some embodiments, the D-peptidic variant Z domain polypeptide includes a sequence having 80% or more sequence identity with a sequence of 979268 (SEQ ID NO: 32), such as 85% or more, 87% or more, 89% or more, 91% or more, 93% or more, 94% or more, 96% or more, 98% or more sequence identity. In some embodiments, the D-peptidic variant Z domain polypeptide includes a sequence having 80% or more sequence identity with a sequence of 979269 (SEQ ID NO: 33), such as 85% or more, 87% or more, 89% or more, 91% or more, 93% or more, 94% or more, 96% or more, 98% or more sequence identity.

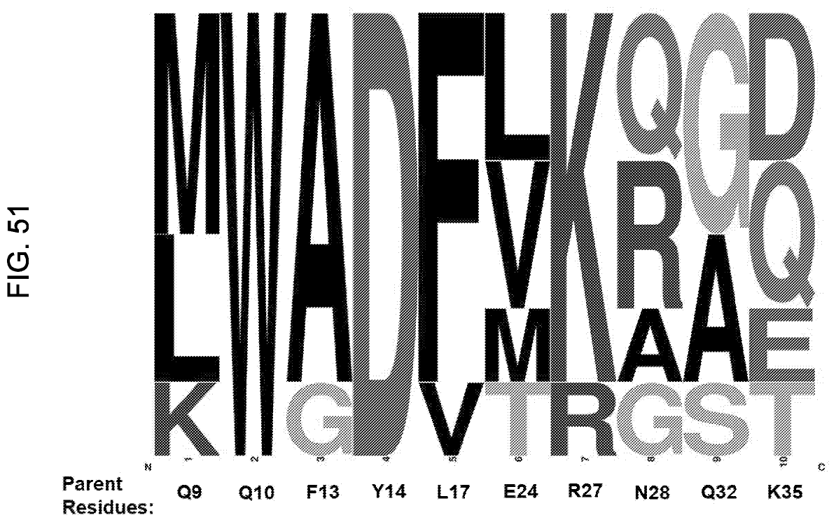

The PD-1 binding D-peptidic variant Z domain polypeptides can have amino acid residues at positions 9, 10, 13, 14, 17, 24, 27, 28, 32 and 35 of a Z domain scaffold that are defined by the specificity-determining motif (SDM) depicted in FIG. 4A and FIG. 51. In some embodiments, the specificity-determining motif (SDM) is defined by the following sequence motif:

| (SEQ ID NO: 72) | |
|---|---|
| | x⁹w¹⁰--x¹³d¹⁴--x¹⁷------x²⁴--x²⁷x²⁸---x³²--x³⁵ |

wherein: x⁹, x¹³, x¹⁷, x²⁴, x²⁷, x²⁸ x³² and x³⁵ are each independently any amino acid residue. In certain cases of the SDM:

x⁹ is selected from k, l and m;

x¹³ is selected from a and G;

x¹⁷ is selected from f and v;

x²⁴ is selected from l, m, t and v;

x²⁷ is selected from k and r;

x²⁸ is selected from a, G, q and r;

x³² is selected from a, G and s; and

x³⁵ is selected from d, e, q and t.

In certain cases, the specificity-determining motif (SDM) is:

m⁹w¹⁰--a¹³d¹⁴--f¹⁷------x²⁴--k²⁷x²⁸---x³²--x³⁵

wherein x²⁴, x²⁸, x³² and x³⁵ are each independently any amino acid residue. Alternatively, the specificity-determining motif (SDM) is: x⁹w¹⁰-x¹³d¹⁴-x¹⁷⁻-t²⁴-x²⁷r²⁸-G³²-q³⁵
wherein x⁹, x¹³, x¹⁷ and x²⁷ are each independently any amino acid residue. In certain cases, the specificity-determining motif (SDM) is: m⁹w¹⁰-a¹³d¹⁴-f¹⁷-t²⁴-k²⁷-r²⁸-G³²-q³⁵.

In some embodiments, D-peptidic Z domain includes: a) a PD-1 specificity-determining motif (SDM) defined by the following amino acid residues:

(SEQ ID NO: 72)
x⁹w¹⁰--x¹³d¹⁴--x¹⁷------x²⁴--x²⁷x²⁸---x³²--x³⁵

wherein:
x⁹ is selected from k, l and m;
x¹³ is selected from a and G;
x¹⁷ is selected from f and v;
x²⁴ is selected from k, l, m, r, t and v;
x²⁷ is selected from k and r;
x²⁸ is selected from a, G, q, r and s;
x³² is selected from a, G and s; and
x³⁵ is selected from d, e, q and t.

In some embodiments, the PD-1 SDM is defined as having 80% or more, or 90% or more identity with the SDM residues defined in (a) as shown above (e.g. x⁹w¹⁰-x¹³d¹⁴-x¹⁷-x²⁴-x²⁷ x²⁸-x³²-x³⁵ (SEQ ID NO: 72)); In some embodiments, the PD-1 SDM is defined as having c) a PD-1 SDM having 1 to 3 amino acid residue substitutions relative to the SDM residues defined in (a) as shown above (e.g. x⁹w¹⁰-x¹³d¹⁴-x¹⁷-x²⁴-x²⁷x²⁸-x³²-x³⁵ (SEQ ID NO: 72)), wherein the 1 to 3 amino acid residue substitutions are selected from: i) a similar amino acid residue substitution according to Table 1; ii) a conservative amino acid residue substitution according to Table 1; iii) a highly conserved amino acid residue substitution according to Table 1; and iv)

an amino acid residue substitution according to the SDM defined in FIG. 4A or FIG. 51.

In some embodiments, the SDM residues defined in (a) as shown above (e.g. x⁹w¹⁰-x¹³d¹⁴-x¹⁷-x²⁴-x²⁷x²⁸-x³²-x³⁵ (SEQ ID NO: 72)) are:

m⁹w¹⁰--x¹³d¹⁴--f¹⁷------x²⁴--k²⁷x²⁸---x³²--x³⁵;
or
m⁹w¹⁰--a¹³d¹⁴--f¹⁷------x²⁴--k²⁷x²⁸---x³²--x³⁵;
or
x⁹w¹⁰--x¹³d¹⁴--x¹⁷------t²⁴--x²⁷r²⁸---G³²--q³⁵

wherein:
x⁹ is selected from k, l and m;
x¹³ is selected from a and G;
x¹⁷ is selected from f and v;
x²⁴ is selected from k, r and t;
x²⁷ is selected from k and r;
x²⁸ is selected from r and s;
x³² is selected from a and G; and
x³⁵ is selected from d and q.

In some embodiments, the SDM residues defined in (a) as shown above (e.g. x⁹w¹⁰-x¹³d¹⁴-x¹⁷-x²⁴-x²⁷x²⁸-x³²-x³⁵ (SEQ ID NO: 72)) are:

m⁹w¹⁰--a¹³d¹⁴--f¹⁷------t²⁴--k²⁷r²⁸---G³²--q³⁵
or
m⁹w¹⁰--G¹³d¹⁴--f¹⁷------r²⁴--k²⁷s²⁸---a³²--d³⁵
or
m⁹w¹⁰--G¹³d¹⁴--f¹⁷------t²⁴--k²⁷r²⁸---G³²--q³⁵
or
m⁹w¹⁰--G¹³d¹⁴--f¹⁷------k²⁴--k²⁷r²⁸---a³²--q³⁵.

In some embodiments, the PD-1 SDM is defined by the following residues:

m⁹w¹⁰--a¹³d¹⁴--f¹⁷------t²⁴--k²⁷r²⁸---G³²--q³⁵

In some embodiments, the PD-1 SDM is defined by the following residues:

m⁹w¹⁰--G¹³d¹⁴--f¹⁷------r²⁴--k²⁷s²⁸---a³²--d³⁵
or
m⁹w¹⁰--G¹³d¹⁴--f¹⁷------t²⁴--k²⁷r²⁸---G³²--q³⁵
or
m⁹w¹⁰--G¹³d¹⁴--f¹⁷------k²⁴--k²⁷r²⁸---a³²--q³⁵.

In some embodiments, the SDM residues are comprised in a polypeptide including: a) D-peptidic framework residues defined by the following amino acid residues: -n¹¹a-e¹⁵i-h¹⁸lpnln-e²⁵q-a²⁹fi-s³³l-. In some embodiments, the D-peptidic framework residues are define by having 80% or more (e.g., 90% or more) identity with the residues defined in (a) as shown above (e.g. -n¹¹a-e¹⁵i-h¹⁸lpnln-e²⁵q-a²⁹fi-s³³l-); or c) peptidic framework residues having 1 to 3 amino acid residue substitutions relative to the residues defined in (a) as shown above (e.g. -n¹¹a-e¹⁵i-h¹⁸lpnln-e²⁵q-a²⁹fi-s³³l-), wherein the 1 to 3 amino acid residue substitutions are selected from: i) a similar amino acid residue substitution according to Table 1; ii) a conservative amino acid residue substitution according to Table 1; and iii) a highly conserved amino acid residue substitution according to Table 1.

In some embodiments, a SDM-containing sequence has 80% or more (e.g., 85% or more, 90% or more, or 95% or more) identity to the amino acid sequence:
x⁹wnax¹³deix¹⁷hlpnlnx²⁴ x²⁷ x²⁸afix³²slx³⁵ (SEQ ID NO: 57), wherein:

x⁹ is selected from k, l and m;

x¹³ is selected from a and G;

x¹⁷ is selected from f and v;

x²⁴ is selected from k, l, m, r, t and v;

x²⁷ is selected from k and r;

x²⁸ is selected from a, G, q, r and s;

x³² is selected from a, G and s; and

x³⁵ is selected from d, e, q and t.

In some embodiments, the D-peptidic Z domain includes a three-helix bundle of the structural formula:

[Helix 1^{(#8-18)}]-[Linker 1^{(#19-24)}]-[Helix 2^{(#25-36)}]-[Linker 2^{(#37-40)}]-[Helix 3^{(#41-54)}]

wherein: # denotes reference positions of amino acid residues comprised in the D-peptidic Z domain; and Helix 3^{(#41-54)} includes a D-peptidic framework sequence selected from: a) s⁴¹anllaeakklnda⁵⁴ (SEQ ID NO: 58); b) a sequence having 70% or more (e.g., 75% or more, 80% or more, 85% or more, or 90% or more) identity to the amino acid sequence set forth in (a); or c) a sequence having 1 to 5 amino acid residue substitutions relative to the sequence defined in (a), wherein the 1 to 5 amino acid residue substitutions are selected from: i) a similar amino acid residue substitution according to Table 1; ii) a conservative amino acid residue substitution according to Table 1; and iii) a highly conserved amino acid residue substitution according to Table 1.

In some embodiments, the D-peptidic Z domain further includes a C-terminal D-peptidic framework sequence having 70% or more (e.g., 75% or more, 80% or more, 85% or more, or 90% or more) identity with the amino acid sequence: d³⁶dpsgsanllaeakklndaqapk⁵⁸ (SEQ ID NO: 59).

In some embodiments, the D-peptidic Z domain further includes an N-terminal D-peptidic framework sequence selected from: a) v¹dnx⁴fnx⁷e⁸ (SEQ ID NO: 60);

wherein:
x⁴ is k, n, r or s; and
x is k or i.

In some embodiments, the D-peptidic Z domain further includes a sequence having 60% or more (e.g., 75% or more, 85% or more) sequence identity relative to the one or more segments defined in (a) as shown above (e.g. v¹dnx⁴fnx⁷e⁸ (SEQ ID NO: 60).

In some embodiments, the N-terminal D-peptidic framework sequence is selected from:

(SEQ ID NO: 61)
v¹dnkfnke⁸;
(SEQ ID NO: 62)
v¹dnnfnie⁸;
(SEQ ID NO: 63)
v¹dnrfnie⁸;
and
(SEQ ID NO: 64)
v¹dnsfnie⁸.

In some embodiments, the D-peptidic Z domain includes: a) a sequence selected from one of compounds 978060 to 978065 (SEQ ID NOs: 36-41), 979259 to 979262 (SEQ ID NOs: 24-27), and 979264 to 979269 (SEQ ID NOs: 28-33), and 981195 to 981197 (SEQ ID NOs: 42-44); b) a sequence having 80% or more identity with the sequence defined in (a); or c) a sequence having 1 to 10 (e.g., 1 to 6, 1 to 5, 1 to 4, 1 to 3, 1 to 2, 2 or 1) amino acid residue substitutions relative to the sequence defined in (a), wherein the 1 to 10 amino acid substitutions are selected from: i) a similar amino acid residue substitution according to Table 1; ii) a conservative amino acid residue substitution according to Table 1; and iii) a highly conserved amino acid residue substitution according to Table 1.

In some embodiments, the D-peptidic Z domain includes a polypeptide of one of compounds 978060 to 978065 and 981195 to 981197 (SEQ ID NOs: 36-41). In some embodiments, the D-peptidic Z domain includes a polypeptide of one of compounds 979259 to 979262 (SEQ ID NOs: 24-27), 979264 to 979269 (SEQ ID NOs: 28-33).

Also provided are D-peptidic compounds that have been optimized for binding affinity and specificity to target protein by affinity maturation, e.g., second, third or fourth or higher generation D-peptidic compounds based on a parent compound that binds to target protein. In some embodiments, the affinity maturation of a subject compound may include holding a fraction of the variant amino acid positions as fixed positions while the remaining variant amino acid positions are varied to select optimal amino acids at each position. A parent D-peptidic compound may be selected as a scaffold for an affinity maturation compound. In some embodiments, a number of affinity maturation compounds are prepared that include mutations at limited subsets of the variant amino acid positions of the parent, while the rest of the variant positions are held as fixed positions. The positions of the mutations may be tiled through the scaffold sequence to produce a series of compounds such that mutations at every variant position are represented and a diverse range of amino acids are substituted at every position (e.g., all 20 naturally occurring amino acids). Mutations that include deletion or insertion of one or more amino acids may also be included at variant positions of the affinity maturation compounds. An affinity maturation compound may be prepared and screened using any convenient method, e.g., phage display library screening, to identify second generation compounds having an improved property, e.g., increased binding affinity for a target molecule, protein folding, protease stability, thermostability, compatibility with a pharmaceutical formulation, etc.

In some embodiments, the affinity maturation of a subject compound may include holding most or all of the variant amino acid positions in the variable regions of the parent compound as fixed positions, and introducing contiguous mutations at positions adjacent to these variable regions. Such mutations may be introduced at positions in the parent compound that were previously considered fixed positions in the original GA scaffold domain. Such mutations may be used to optimize the compound variants for any desirable property, such as protein folding, protease stability, thermostability, compatibility with a pharmaceutical formulation, etc.

### Exemplary Multivalent D-Peptidic Compounds

This disclosure provides multivalent compounds that bind PD-1. The multivalent PD-1 binding compound can be bivalent and include two distinct variant domains connected via a linking component (e.g., as described herein).

In some embodiments, a multivalent D-peptidic compound of the present disclosure includes a first D-peptidic domain that specifically binds a target protein; and a second D-peptidic domain that specifically binds the target protein and is heterologous to the first D-peptidic domain; and a linking component that covalently links the first and second D-peptidic domains. In some embodiments, the second D-peptidic domain specifically binds the target protein at a distinct binding site on the target protein that is non-overlapping with the binding site bound by the first D-peptidic domain. In some embodiments, the linking component covalently links the first and second D-peptidic domains such that the first and second D-peptidic domains are capable of simultaneously binding the target protein.

In some embodiments, the D-peptidic domains are configured as a dimer of a bivalent moiety including first and second D-peptidic domains.

In some embodiments, the target protein is monomeric. In some embodiments, the target protein is dimeric. In some embodiments, the target protein is PD-1.

In some embodiments, the multivalent D-peptidic compound of the present disclosure includes a first D-peptidic domain that is a first three-helix bundle domain capable of specifically binding a first binding site of the target protein; and a second D-peptidic domain that is a second three-helix bundle domain capable of specifically binding a second binding site of the target protein.

In some embodiments, the first and second D-peptidic domains specifically bind to distinct non-overlapping binding sites of the target protein. In some embodiments, the compound is bivalent.

In some embodiments, the first binding site is non-overlapping with the PD-L1 binding site on PD-1. In some embodiments, the first binding site includes the amino acid sidechains S38, P39, A40, T53, S55, L100, P101, N102, R104, D105 and H107 of PD-1.

In some embodiments, the second binding site overlaps at least partially with the PD-L1 binding site on PD-1. In some embodiments, the second binding site includes the amino acid sidechains V64, N66, Y68, M70, T76, K78, Ii26, L128, A132, Q133, I134 and E136 of PD-1.

In some embodiments, the first D-peptidic domain is linked to the second D-peptidic domain via a N-terminal to N-terminal linker. In some embodiments, the N-terminal to N-terminal linker is a (PEG)ₙ bifunctional linker, wherein n is 2-20 (e.g., n is 3-12 or 6-8, such as 3, 4, 5, 6, 7, 8, 9 or 10).

In some embodiments, the first D-peptidic domain is a first three-helix bundle domain capable of specifically binding a first binding site of the target protein; and the second D-peptidic domain is a second three-helix bundle domain capable of specifically binding a second binding site of the target protein.

In some embodiments, the first and second D-peptidic domains are selected from D-peptidic GA domain and D-peptidic Z domain. In some embodiments, the first D-peptidic domain is a D-peptidic GA domain; and the second D-peptidic domain is a D-peptidic Z domain.

In some embodiments, the first D-peptidic domain is a D-peptidic GA domain polypeptide having a specificity-determining motif (SDM) including 5 or more (e.g., 5, 6, 7, 8, 9, 10, 11, 12, 13, 14, 15 or 16) variant amino acid residues at positions selected from 25, 27, 30, 31, 34, 36, 37, 39, 40 and 42-48. In some embodiments, the GA domain includes a polypeptide of the sequence: tidgwllknakedaiaelkkaGitsdlyfnwinvaGsvssvnfhknyilkaha (SEQ ID NO: 32).

In some embodiments, the second D-peptidic domain is a D-peptidic Z domain having a specificity-determining motif (SDM) comprising 5 or more variant amino acid residues (e.g., 6 or more, such as 6, 7, 8, 9 or 10) at positions selected from 9, 10, 13, 14, 17, 24, 27, 28, 32 and 35. In some embodiments, the D-peptidic Z domain includes a polypeptide of the sequence: vdnkfnkemwnaadeifhlpnlnteqkrafiGslqddpsgsanllaeakklndaqapk (SEQ ID NO: 40).

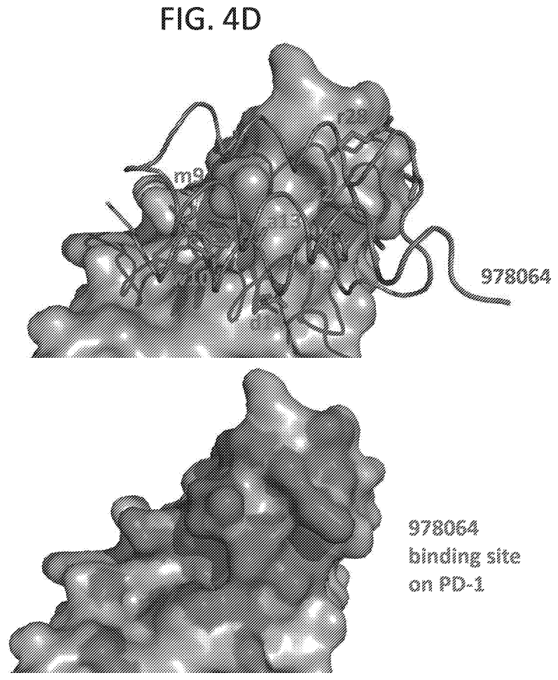

Exemplary single D-peptidic domains that specifically bind PD-1 are disclosed herein that bind to one of two different binding sites on the target protein. FIG. 7A-7B shows the crystal structures of two such single domains simultaneous bound to target PD-1. PD-1 specific variant GA domain polypeptides are described herein that bind at a first binding site of PD-1. In some embodiments, the first binding site is defined by the amino acid sidechains S38, P39, A40, T53, S55, L100, P101, N102, R104, D105 and H107 of PD-1. In some embodiments, PD-1 specific polypeptide is a locked variant GA domain. Any of the subject PD-1 specific D-peptidic variant GA domain polypeptides can be connected via a linking component to a second D-peptidic domain that specifically binds to a second and distinct binding site of the target PD-1. In some case, the second binding site is defined by the amino acid sidechains V64, N66, Y68, M70, T76, K78, 1126, L128, A132, Q133, 1134 and E136 of PD-1. See FIG. 7A showing exemplary Z domain polypeptide 978064 binding at a site distinct from the exemplary GA domain polypeptide 977296. At least one or both of the target binding sites should partially overlap the PD-L1 binding site on the PD-1 target protein in order to provide antagonist activity. See e.g., FIG. 7B.

D-peptidic variant GA domain polypeptides which can be linked to a D-peptidic variant Z domain polypeptide in order to provide a PD-1 binding bivalent compound include, but are not limited to, compounds 977296-977299, 977978-977979, and variants thereof (e.g., as described herein).

D-peptidic variant Z domain polypeptides which can be linked to a D-peptidic variant GA domain polypeptide in order to provide a PD-1 binding bivalent compound include, but are not limited to, compounds 978060-978065, 979259 to 979262, 979264 to 979269, and 981195-981197, and variants thereof (e.g., as described herein).

D-peptidic variant Z domain polypeptides which can be linked to a D-peptidic variant GA domain polypeptide in order to provide a PD-1 binding bivalent compound include, but are not limited to, compounds 978060-978065, 979259 to 979262, 979264 to 979269, and 981195-981197, and variants thereof (e.g., as described herein). For example, Table 3 provides details of exemplary bivalent compounds that bind PD-1 with high affinity, compounds 979820, 979821 979450, 981851, 980861, 982007, and 982864.

In some embodiments, the D-peptidic compound specifically binds the target protein with a binding affinity (KD) 10-fold or more (e.g., 30-fold or more, 100-fold or more, 300-fold or more or 1000-fold or more, as measured by SPR) stronger than each of the binding affinities of the first and second D-peptidic domains alone for the target protein.

In some embodiments, the compound has a binding affinity (KD) for the target protein of 3 nM or less (e.g., 1 nM or less, 300 μM or less, 100 μM or less); and the binding affinities of the first and second D-peptidic domains alone for the target protein are each independently 100 nM or more (e.g., 300 nM or more, 1 uM or more).

In some embodiments, the D-peptidic compound has in vitro antagonist activity (IC50) against the target protein that is at least 10-fold more potent (e.g., at least 30-fold, at least 100-fold, at least 300-fold, etc. as measured by ELISA assay as described herein) than each of the first and second D-peptidic domains alone.

In some embodiments, the first D-peptidic domain consists essentially of a single chain polypeptide sequence of 30 to 80 residues (e.g., 40 to 70, 45 to 60 residues, 50 to 60 residues, or 52 to 58 residues), and has a MW of 1 to 10 kDa (e.g., 2 to 8 kDa, 3 to 8 kDa or 4 to 6 kDa). In some embodiments, the second D-peptidic domain consists essentially of a single chain polypeptide sequence of 30 to 80 residues (e.g., 40 to 70, 45 to 60 residues, 50 to 60 residues, or 52 to 58 residues), and has a MW of 1 to 10 kDa (e.g., 2 to 8 kDa, 3 to 8 kDa or 4 to 6 kDa).

In some embodiments, the multivalent D-peptidic compound includes a linking component. In some embodiments, the linking component is a linker connecting a terminal amino acid residue of the first D-peptidic domain to a terminal amino acid residue of the second D-peptidic domain (e.g., N-terminal to N-terminal linker or C-terminal to C-terminal linker). In some embodiments, the linking component is a linker connecting an amino acid sidechain of the first D-peptidic domain to a terminal amino acid residue of the second D-peptidic domain that are in proximity to each other when the first and second D-peptidic domains are simultaneously bound to the target protein. In some embodiments, the linking component is a linker connecting an amino acid sidechain of the first D-peptidic domain to a proximaln amino acid sidechain of the second D-peptidic domain that is proximal to the amino acid sidechain when the first and second D-peptidic domains are simultaneously bound to the target protein.

In some embodiments, the linking component includes one or more groups selected from amino acid residue, polypeptide, (PEG)ₙ linker (e.g., n is 2-50, 3-50, 4-50, 6-50 or 6-20), modified PEG moiety, C₍₁₋₆₎alkyl linker, substituted C₍₁₋₆₎alkyl linker, —CO(CH₂)ₘCO—, —NR(CH₂)ₚNR—, —CO(CH₂)ₘNR—, —CO(CH₂)ₘO—, —CO(CH₂)ₘS—, and linked chemoselective functional groups (e.g., —CONH—, —OCONH—, click chemistry conjugate such as 1,2,3-triazole, maleimide-thiol conjugate thiosuccinimide, haloacetyl-thiol conjugate thioether, etc.), wherein m is 1 to 6, p is 2-6 and each R is independently H, C₍₁₋₆₎alkyl or substituted C₍₁₋₆₎alkyl.

Linking Components that Link GA Domain and Z Domain

In some embodiments, a multivalent D-peptidic compound that specifically binds PD-1 includes a D-peptidic GA domain capable of specifically binding a first binding site of PD-1; and a D-peptidic Z domain capable of specifically binding a second binding site of PD-1.

In some embodiments, the linking component covalently links the D-peptidic GA and Z domains. In some embodiments, the linking component is configured to link the D-peptidic GA and Z domains whereby the domains are capable of simultaneously binding to PD1. In some embodiments, the linking component is configured to connect the D-peptidic GA and Z domains via sidechain and/or terminal groups that are proximal to each other when the D-peptidic GA and Z domains are simultaneously bound to PD1.

In some embodiments, the linking component includes a linker connecting a terminal of the D-peptidic GA domain to a terminal of the D-peptidic Z domain. In some embodiments, the linker connects the N-terminal residue of the D-peptidic GA domain polypeptide to the N-terminal residue of the D-peptidic Z domain polypeptide.

In some embodiments, the linking component connects a first amino acid sidechain of a residue of the D-peptidic GA domain and a second amino acid sidechain of a residue of the D-peptidic Z domain. In some embodiments, the linking component includes one or more groups selected from amino acid residue, polypeptide, (PEG)ₙ linker (e.g., n is 2-50, 3-50, 4-50, 6-50 or 6-20), modified PEG moiety, C₍₁₋₆₎alkyl linker, substituted C₍₁₋₆₎alkyl linker, —CO(CH₂)ₘCO—, —NR(CH₂)ₚNR—, —CO(CH₂)ₘNR—, —CO(CH₂)ₘO—, —CO(CH₂)ₘS—, and linked chemoselective functional groups (e.g., —CONH—, —OCONH—, click chemistry conjugate such as 1,2,3-triazole, maleimide-thiol conjugate thiosuccinimide, haloacetyl-thiol conjugate thioether, etc.), wherein m is 1 to 6, p is 2-6 and each R is independently H, C₍₁₋₆₎alkyl or substituted C₍₁₋₆₎alkyl.

In some embodiments, the D-peptidic GA domain and the D-peptidic Z domain are conjugated to each other via N-terminal cysteine residues with a bis-maleimide linker or bis-haloacetyl linker, optionally comprising a (PEG)ₙ moiety (e.g., n is 2-12, such as 3-8, e.g., a PEG3, PEG6, or PEG8 containing linker).

In some embodiments, the linking component connecting the D-peptidic GA and Z domains is selected from:

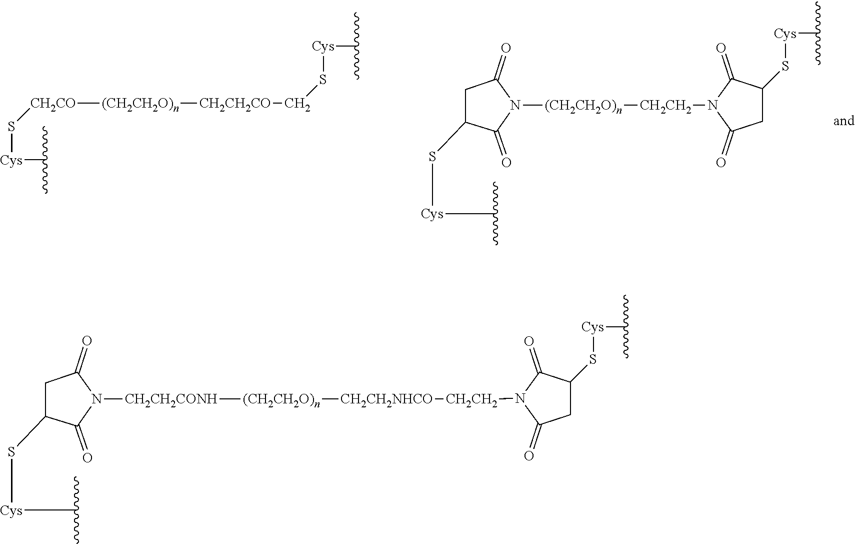

wherein n is 1-20 (e.g., 2 to 12, 2 to 8, or 3 to 6).

### Exemplary Multimeric Multivalent D-Peptidic Compounds

Aspects of this disclosure include multimeric (e.g., dimeric, trimeric or tetrameric, etc) D-peptidic compounds that include any two or more of the subject variant domain polypeptides and/or bivalent compounds described herein.

In some embodiments, the multivalent D-peptidic compound includes a first D-peptidic domain that specifically binds a target protein; a second D-peptidic domain that specifically binds the target protein and is heterologous to the first D-peptidic domain; and a third D-peptidic domain that specifically binds a target protein (e.g., trivalent, tetravalent, etc.).

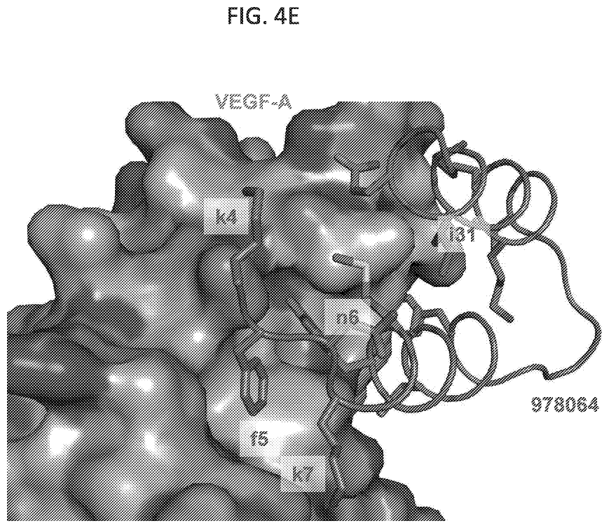

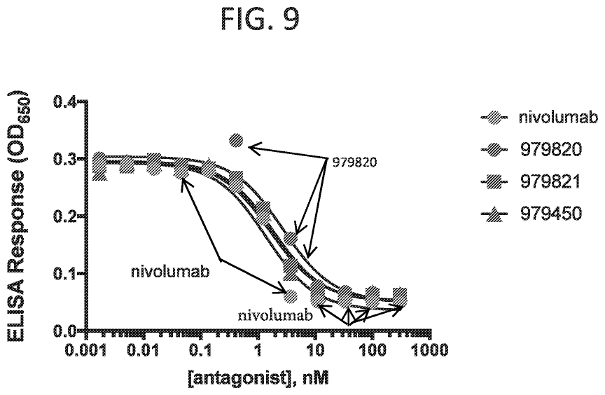

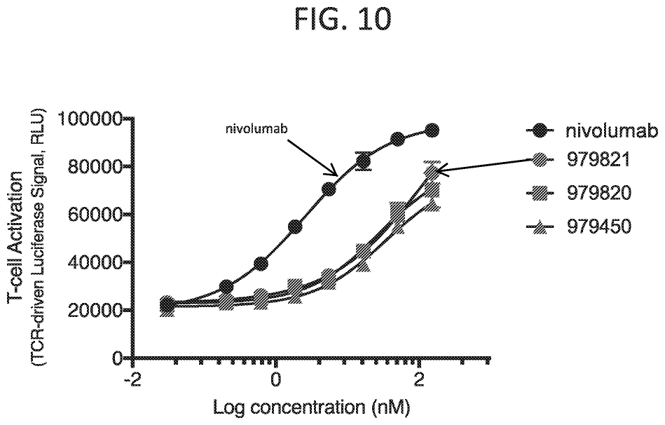

A multimer of the present disclosure can refer to a compound having two or more homologous domains or two or more homologous bivalent compounds. As such, a dimer of a bivalent compound can include two molecules of any one of the bivalent compounds described herein, connected via a linking component. When the target molecule is a PD-1 homodimer, a homologous dimeric compound can provide for binding to analogous sites on each PD-1 target monomer. For example, FIG. 7A shows an overlay of the crystal structures of two molecules of domain 977296 and domain 978064 bound to PD-1. Exemplary sites for incorporating chemical linkages to connect the domains are indicated in FIG. 8A. Exemplary linking components are elaborated in FIGS. 8A and 8C. In some embodiments, dimerization of the multimeric compound (978064+977296) is achieved using a peptidic linker between the C-terminals. For example, Table 3 and FIG. 14A-B show the sequences and configuration of exemplary PD-1 binding dimeric bivalent compounds 978064 and 977296. Any convenient linking groups may be linked to the C-terminal of a polypeptide domain to introduce a dimerizing linking component, either during SPPS or post SPPS (e.g., as described herein).

In some embodiments, the multivalent D-peptidic compound of the present disclosure includes a first D-peptidic domain, a second D-peptidic domain, and third D-peptidic domain that is homologous to the first D-peptidic domain. In some embodiments, the multivalent D-peptidic compound of the present disclosure includes a fourth D-peptidic domain that is homologous to the second D-peptidic domain.

In some embodiments, multimeric multivalent D-peptidic compounds of the present disclosure includes the following polypeptides:

tidgwllknakedaiaelkkaGitsdlyfnwinvaGsvssvnfhknyilkaha (SEQ ID NO: 65); and vdnkfnkemwnaadeifhlpnlnteqkrafiGslqddpsgsanllaeakklndaqapk (SEQ ID NO: 66). In some embodiments, the polypeptides are linked via N-terminal cysteine residues with a bis-maleimide bifunctional linking moiety including PEG3, PEG6 or PEG8. further includes a second GA domain that is homologous to the first GA domain. In some embodiments, the compound further includes a second Z domain that is homologous to the first Z domain.

A multimeric compound of this disclosure can alternatively be heterologous. As such, a multimeric compound can include two or more domains and/or bivalent compounds that target two different target proteins, e.g., a bispecific dimeric compound. In some embodiments, one of the target proteins is PD-1. In certain cases, one of the target proteins is VEGF-A. In certain instances, the multimeric compound can further target a second protein such as CD3. Combinations of target proteins that can be targeted using the subject multimeric compounds include PD-1 and CD3, and VEGF-A and CD3. Sometimes, the compound may be referred to as a D-peptidic bispecific T cell engager.

TABLE 1
Exemplary D-peptidic Domain Scaffolds

| Peptidic | | SEQ ID |
|---|---|---|
| Domain | Sequence | NO |
| Z | vdnkfnkeqqnafyeilhlpnlneeqrnafiqslkddpsqsanllaeakk | 1 |
| Domain | lndaqapk | |
| GA | tidqwllknakedaiaelkkaGitsdfyfnainkaktveevnalkneilk | 2 |
| Domain | aha | |
| GA | ......l⁷..a¹⁰ke.ai.elk.²⁰.Gi.sd.y..³⁰.inkaktve.⁴⁰v.al | 3 |
| consensus | k.eil⁴⁹.... | |
| ALB8-GA | t¹idqwll⁷knakedaiaelkkaGitsdfyfnainkaktveevnalkneil | 4 |
| | kaha⁵³ | |
| ALB1-GA | l⁷knakedaiaelkkaGitsdfyfnainkaktveGanalkneilka⁵¹ | 5 |
| ALB8- | l⁷kltkeeaekalkklGitsefilnqidkatsreGleslvqtikqs⁵¹ | 6 |
| uGA | | |
| ALB1B- | l⁷qeakdkaiqeakanGltsklllknienaktpesaksfaeeliks⁵¹ | 7 |
| uGA | | |
| L3316- | l⁷knakeeaikelkeaGitsdlyfslinkaktveGvealkneilka⁵¹ | 8 |
| GA1 | | |
| L3316- | l⁷knakedaikelkeaGissdiyfdainkaktveGvealkneilka⁵¹ | 9 |
| GA2 | | |
| L3316- | l⁷knakeaaikelkeaGitaeylfnlinkaktveGveslkneilka⁵¹ | 10 |
| GA3 | | |
| L3316- | l⁷knakedaikelkeaGitsdiyfdainkaktieGvealkneilka⁵¹ | 11 |
| GA4 | | |
| G148- | l⁷akakadalkefnkyGv- | 12 |
| GA1 | sdyyknlinnaktveGvkdlqaqvves⁵¹ | |
| G148- | l⁷aeakvlanreldkyGv- | 13 |
| GA2 | sdyhknlinnaktveGvkdlqaqvves⁵¹ | |
| G148- | l⁷aeakvlanreldkyGv- | 14 |
| GA3 | sdyyknlinnaktveGvkalideilaalp⁵³ | |
| DG12- | l⁷dnaknaalkefdryGv- | 15 |
| GA1 | sdyyknlinkaktveGimelqaqvves⁵¹ | |
| DG12- | l⁷seakemaireldanGv- | 16 |
| GA2 | sdfykdkiddaktveGvvalkdlilns⁵¹ | |
| MAG-GA1 | l⁷aklaadtdldldvakiind- | 17 |
| | yttkvenaktaedvkkifee--sq⁵¹ | |
| MAG-GA2 | l⁷akakadaieilkkyGi- | 18 |
| | GdyyiklinnGktaeGvtalkdeil--⁵¹ | |
| ZAG-GA | l⁷leakeaainelkqyGi- | 19 |
| | sdyyvtlinkaktveGvnalkaeilsa⁵¹ | |

TABLE 2
Exemplary Variant D-Peptidic Domain that bind target proteins

| | | | Binding | |
|---|---|---|---|---|
| Compound | Target | | Affinity | SEQ ID |
| # | Protein | Sequence | K_{D} (nM) | NO |
| GA | | tidqwllknakedaiaelkkaGitsdfyfnainkaktve | No | 20 |
| domain | | Helix 1 Helix 2 | binding | |
| wt | | evnalkneilkaha | | |
| | | Helix 3 | | |
| 977296 | PD-1 | tidqwllknakedaiaelkkaGitsdlyfnwinvaGsvs | 1507 | 32 |
| | | svnfhknyilkaha | | |
| 977297 | PD-1 | tidqwllknakedaiaelkkaGitsdlyfnwinvaGsvs | 2950 | 33 |
| | | svnyhknfilkaha | | |
| 977298 | PD-1 | tidqwllknakedaiaelkkaGitsdlyfnwinvaGsvs | 871 | 34 |
| | | svnyhknyilkaha | | |
| 977299 | PD-1 | tidqwllknakedaiaelkkaGitsdlyfnwindassvs | 6480 | 35 |
| | | svnfhknyilkaha | | |
| 977978 | PD-1 | tidqwllknakedaiaelkkaGitcdlyfnwinvaGsvs | 114 | 21 |
| | | svnfhknyilkaha | | |
| 977979 | PD-1 | tidqwllknakedaiaelkkaGitsdlyfnwinvassvs | 536 | 22 |
| | | svnfhknyilkaha | | |
| Z | | vdnkfnkeqqnafyeilhlpnlneeqrnafiqslkddps | No | 23 |
| domain | | Helix 1 Helix 2 | binding | |
| wt | | qsanllaeakklndaqapk | | |
| | | Helix 3 | | |
| 978060 | PD-1 | vdnkfnkekwnaadeifhlpnlnveqkaafissleddps | 1800 | 36 |
| | | qsanllaeakklndaqapk | | |
| 978061 | PD-1 | vdnkfnkelwnaadeifhlpnlnleqkqafiGsldddps | 1340 | 37 |
| | | qsanllaeakklndaqapk | | |
| 978062 | PD-1 | vdnkfnkelwnaadeivhlpnlnleqrrafiasltddps | 4430 | 38 |
| | | qsanllaeakklndaqapk | | |
| 978063 | PD-1 | vdnkfnkemwnaadeifhlpnlnmeqkqafiGsldddps | 3600 | 39 |
| | | qsanllaeakklndaqapk | | |
| 978064 | PD-1 | vdnkfnkemwnaadeifhlpnlnteqkrafiGslqddps | 904 | 40 |
| | | qsanllaeakklndaqapk | | |
| 978065 | PD-1 | vdnkfnkemwnaGdeifhlpnlnveqkGafiaslqddps | 2840 | 41 |
| | | qsanllaeakklndaqapk | | |
| 979259 | PD-1 | vdnkfnkemwnaadeifhlpnlnkiqkraficslqddps | 2070 | 24 |
| | | qsanllaeakklndaqapk | | |
| 979260 | PD-1 | vdnkfnkemwnaadeifhlpnlnkiqkraficslqddps | 372 | 25 |
| | | qsanllaeakklndaqapk | | |
| 979261 | PD-1 | vdnkfnkemwnaadeifhlpnlntvqkraficslqddps | 6 | 26 |
| | | qsanllaeakklndaqapk | | |
| 979262 | PD-1 | vdnkfnkemwnaadeifhlpnlntvqkraflcslqddps | 349 | 27 |
| | | qsanllaeakklndaqapk | | |
| 979264 | PD-1 | vdnkfnkemwnaadeifhlpnlnilqkraficslqqdps | 5 | 28 |
| | | qsanllaeakklndaqapk | | |
| 979265 | PD-1 | vdnkfnkemwnaadeifhlpnlntvqkraficslqqdps | 7.6 | 29 |
| | | qsanllaeakklndaqapk | | |
| 979266 | PD-1 | vdnkfnkemwnaadeifhlpnlntyqkraficslqqdps | 16 | 30 |
| | | qsanllaeakklndaqapk | | |
| 979267 | PD-1 | vdnkfnkemwnaadeifhlpnlnkiqkraficslqqdps | 7.8 | 31 |
| | | qsanllaeakklndaqapk | | |
| 979268 | PD-1 | vdnkfnkemwnaadeifhlpnlnivqkraflcslqqdps | 24 | 32 |
| | | qsanllaeakklndaqapk | | |
| 979269 | PD-1 | vdnkfnkemwnaadeifhlpnlnniqksaficslqqdps | 14 | 33 |
| | | qsanllaeakklndaqapk | | |
| 981195 | PD-1 | vdnnfniemwnaadeifhlpnlnreqksafiasldddps | 391 | 42 |
| | | qsanllaeakklndaqapk | | |
| 981196 | PD-1 | vdnrfniemwnaadeifhlpnlnteqkrafiGslqddps | 229 | 43 |
| | | qsanllaeakklndaqapk | | |
| 981197 | PD-1 | vdns fniemwnaadeifhlpnlnkeqkrafiaslqddps | 278 | 44 |
| | | qsanllaeakklndaqapk | | |

TABLE 3
Exemplary Multivalent D-Peptidic Compounds

| | | | | | Binding | |
|---|---|---|---|---|---|---|
| | | | | | Affinity | SEQ |
| Compound | Target | | Linking | | K_{D} | ID |
| # | Protein | Domain 1 | Component | Domain 2 | (nM) | NO: |
| 979821 | PD-1 | 977296 | Mal-PEG3- | 978064 | 0.29 | 45 |
| | | | Mal | | | |
| | | | N-terminal | | | |
| | | | to N- | | | |
| | | | terminal | | | |
| | | | via | | | |
| | | | cysteine | | | |
| | | | conjugations | | | |
| 979820 | PD-1 | 977296 | Mal-PEG6- | 978064 | 0.41 | 46 |
| | | | Mal | | | |
| | | | N-terminal | | | |
| | | | to N- | | | |
| | | | terminal | | | |
| | | | via | | | |
| | | | cysteine | | | |
| | | | conjugations | | | |
| 979450 | PD-1 | 977296 | Mal-PEG8- | 978064 | 0.59 | 47 |
| | | | Mal | | | |
| | | | N-terminal | | | |
| | | | to N- | | | |
| | | | terminal | | | |
| | | | via | | | |
| | | | cysteine | | | |
| | | | conjugations | | | |
| 981851 | PD-1 | 977979 | Mal-PEG6- | 981196 | 1 | 48 |
| | | | Mal | | | |
| | | | N-terminal | | | |
| | | | to N- | | | |
| | | | terminal | | | |
| | | | via | | | |
| | | | cysteine | | | |
| | | | conjugations | | | |
| 980861 | PD-1 | 977296 | Mal-PEG3- | 2x (979261 | 0.17 | 49 |
| | | | Mal | interdimer | | |
| | | | N-terminal | disulfide | | |
| | | | to N- | | | |
| | | | terminal | | | |
| | | | via | | | |
| | | | cysteine | | | |
| | | | conjugations | | | |
| 982007 | PD-1 | 977296 | PEG3- | 2x (979261 | 0.26 | 50 |
| | | | triazole- | interdimer | | |
| | | | PEG3 | disulfide | | |
| | | | N-terminal | | | |
| | | | to N- | | | |
| | | | terminal | | | |
| | | | via Click | | | |
| | | | conjugation | | | |
| 982864 | PD-1 | 2x (977978 | PEG3- | 2x (979261 | 0.37 | 51 |
| | | interdimer | triazole- | interdimer | | |
| | | disulfide | PEG3 | disulfide | | |
| | | | N-terminal | | | |
| | | | to N- | | | |
| | | | terminal | | | |
| | | | via Click | | | |
| | | | conjugation | | | |

Aspects of the present disclosure include compounds (e.g., as described herein), salts thereof (e.g., pharmaceutically acceptable salts), and/or solvate or hydrate forms thereof. It will be appreciated that all permutations of salts, solvates and hydrates are meant to be encompassed by the present disclosure. In some embodiments, the subject compounds are provided in the form of pharmaceutically acceptable salts. Compounds containing amine and/or nitrogen containing heteraryl groups may be basic in nature and accordingly may react with any number of inorganic and organic acids to form pharmaceutically acceptable acid addition salts. Acids commonly employed to form such salts include inorganic acids such as hydrochloric, hydrobromic, hydriodic, sulfuric and phosphoric acid, as well as organic acids such as para-toluenesulfonic, methanesulfonic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the like salts. In certain specific embodiments, pharmaceutically acceptable acid addition salts include those formed with mineral acids such as hydrochloric acid and hydrobromic acid, and those formed with organic acids such as fumaric acid and maleic acid.

### Compound Properties

The variant D-peptidic domains of the subject multivalent compounds may define a binding surface area of a suitable size for forming protein-protein interactions of high functional affinity (e.g., equilibrium dissociation constant (K_{D})) and specificity (e.g., 300 nM or less, such as 100 nM or less, 30 nM or less, 10 nM or less, 3 nM or less, 1 nM or less, 300 μM or less, or even less). The variant D-peptidic domains may each include a surface area of between 600 and 1800 Å², such as between 800 and 1600 Å², between 1000 and 1400 Å², between 1100 and 1300 Å², or about 1200 Å².

In some embodiments, the multivalent D-peptidic compound specifically binds a target protein with a binding affinity (K_{D}) 10-fold or more stronger, such as 30-fold or more, 100-fold or more, 300-fold or more, 1000-fold or more, or even more, than each of the binding affinities of the first and second D-peptidic domains alone for the target protein. A D-peptidic compound's affinity of a target protein can be determined by any convenient methods, such as using an SPR binding assay or an ELISA binding assay (e.g., as described herein). In certain cases, the multivalent D-peptidic compound has a binding affinity (K_{D}) for the target protein of 3 nM or less, such as 1 nM or less, 300 μM or less, 100 μM or less, and the binding affinities of the first and second D-peptidic domains alone for the target protein are each independently 100 nM or more, such as 200 nM or more, 300 nM or more, 400 nM or more, 500 nM or more, or 1 uM or more. The effective binding affinity of the multivalent D-peptidic compound as a whole may be optimized to provide for a desirable biological potency and/or other property such as in vivo half-life. By selecting individual D-peptidic domains having a particular individual affinities for their target binding site, the overall functional affinity of the multivalent D-peptidic compound can be optimized, as needed.

Potency of the compounds can be assessed using any convenient assays, such as via an ELISA assay measuring IC50 as described in the experimental section herein. In some instances, the subject multivalent compound has in vitro antagonist activity against the target protein that is at least 10-fold more potent, such as at least 30-fold, at least 100-fold, at least 300-fold, at least 1000-fold more potent, than the potency of each of the first and second D-peptidic domains alone.

In certain cases, the target protein is VEGF-A. The subject multivalent compounds may exhibit an affinity (e.g., equilibrium dissociation constant (K_{D})), for VEGF-A of 100 nM or less, such as 30 nM or less, 10 nM or less, 3 nM or less, 1 nM or less, 600 μM or less, 300 μM or less, or even less. In certain cases, the target protein is PD-1. The subject multivalent compounds may exhibit an affinity for PD-1 of 100 nM or less, such as 30 nM or less, 10 nM or less, 3 nM or less, 1 nM or less, 600 μM or less, 300 μM or less, or even less.

The subject D-peptidic compounds may exhibit a specificity for target protein e.g., as determined by comparing the affinity of the compound for the target protein with that for a reference protein (e.g., an albumin protein), where specificity can be a difference in binding affinities by a factor of 10³ or more, such as 10⁴ or more, 10⁵ or more, 10⁶ or more, or even more. In some embodiments, the D-peptidic compounds may be optimized for any desirable property, such as protein folding, proteolytic stability, thermostability, compatibility with a pharmaceutical formulation, etc. Any convenient methods may be used to select the D-peptidic compounds, e.g., structure-activity relationship (SAR) analysis, affinity maturation methods, or phage display methods.

Also provided are D-peptidic compounds that have high thermal stability. In some embodiments, the compounds having high thermal stability have a melting temperature of 50° C. or more, such as 60° C. or more, 70° C. or more, 80° C. or more, or even 90° C. or more. Also provided are D-peptidic compounds that have high protease or proteolytic stability. The subject D-peptidic compounds are resistant to proteases and can have long serum and/or saliva half-lives. Also provided are D-peptidic compounds that have a long in vivo half-life. As used herein, “half-life” refers to the time required for a measured parameter, such the potency, activity and effective concentration of a compound to fall to half of its original level, such as half of its original potency, activity, or effective concentration at time zero. Thus, the parameter, such as potency, activity, or effective concentration of a polypeptide molecule is generally measured over time. For purposes herein, half-life can be measured in vitro or in vivo. In some embodiments, the D-peptidic compound has a half-life of 1 hour or longer, such as 2 hours or longer, 6 hours or longer, 12 hours or longer, 1 day or longer, 2 days or longer, 7 days or longer, or even longer. Stability in human blood may be measured by any convenient method, e.g., by incubating the compound in human EDTA blood or serum for a designated time, quenching a sample of the mixture and analyzing the sample for the amount and/or activity of the compound, e.g., by HPLC-MS, by an activity assay, e.g., as described herein.

Also provided are D-peptidic compounds that have low immunogenicity, e.g., are non-immunogenic. In certain embodiments, the D-peptidic compounds have low immunogenicity compared to an L-peptidic compound. As used herein, low immunogenicity refers to a level of immunogenicity that is 50% or less, such as 40% or less, 30% or less, 20% or less, 10% or less, 5% or less, or 1% or less as compared to a control (e.g., a corresponding L-peptidic compound), as measured according to any convenient assay, such as an immunogenicity assay such as that described by Dintzis et al., “A Comparison of the Immunogenicity of a Pair of Enantiomeric Proteins” Proteins: Structure, Function, and Genetics 16:306-308 (1993).

### Domain Modifications

Any convenient molecules or moieties of interest may be attached to the subject D-peptidic compounds. The molecule of interest may be peptidic or non-peptidic, naturally occurring or synthetic. Molecules of interest suitable for use in conjunction with the subject compounds include, but are not limited to, an additional protein domain, a polypeptide or amino acid residue, a peptide tag, a specific binding moiety, a polymeric moiety such as a polyethylene glycol (PEG), a carbohydrate, a dextran or a polyacrylate, a linker, a half-life extending moiety, a drug, a toxin, a detectable label and a solid support. In some embodiments, the molecule of interest may confer on the resulting D-peptidic compounds enhanced and/or modified properties and functions including, but not limited to, increased water solubility, ease of chemical synthesis, cost, bioconjugation site, stability, isoelectric point (pI), aggregation, reduced non-specific binding and/or specific binding to a second target protein, e.g., as described herein.

In some embodiments of any one of the D-peptidic domain sequences described herein, the polypeptide may be extended to include one or more additional residues at the N-terminal and/or C-terminal of the sequence, such as two or more, three or more, four or more, five or more, 6 or more, or even more additional residues. Such additional residues may be considered part of the D-peptidic domain even though they do not provide a target binding interaction. Any convenient residues may be included at the N-terminal and/or C-terminal of the target binding variant domain to provide for a desirable property or group, such as increased solubility via introduction of a water soluble group, a linkage for conjugation or multimerization, a linkage for connecting the domain to a label or a specific binding moiety.

In some embodiments of any one of the D-peptidic domain sequences described herein, the polypeptide may be truncated to exclude one or more additional residues at the N-terminal and/or C-terminal of the parent sequence, such as 6 or less, 5 or less, 4 or less, 3 or less, 2 or less or one residue.

In some embodiments, the peptidic domain that finds use in the subject multivalent compound is described by formula:

X-L-Z

where X is a peptidic domain (e.g., as described herein); L is an optional linking group; and Z is a molecule of interest, where L is attached to X at any convenient location (e.g., the N-terminal, C-terminal or via the sidechain of a surface residue not involved in binding to the target protein).

The D-peptidic domains and compounds may include one or more molecules of interest, e.g., a N-terminal moiety and/or a C-terminal moiety. In some instances, the molecule of interest is covalently attached via the alpha-amino group of the N-terminal residue, or is covalently attached to the alpha-carboxyl acid group of the C-terminal residue. In other instances, an molecules of interest is attached to the motif via a sidechain group of a residue (e.g., via a c, k, d, e or y residue).

In some embodiments, the D-peptidic compound includes a linking component. In some embodiments, the linking component is a linker connecting a terminal amino acid residue of the first D-peptidic domain to a terminal amino acid residue of a second D-peptidic domain (e.g., N-terminal to N-terminal linker or C-terminal to C-terminal linker). In some embodiments, the linking component is a linker connecting an amino acid sidechain of the first D-peptidic domain to a terminal amino acid residue of the second D-peptidic domain that are in proximity to each other when the first and second D-peptidic domains are simultaneously bound to the target protein.

The molecules of interest may include a polypeptide or a protein domain. Polypeptides and protein domains of interest include, but are not limited to: gD tags, c-Myc epitopes, FLAG tags, His tags, fluorescence proteins (e.g., GFP), beta-galactosidase protein, GST, albumins, immunoglobulins, Fc domains, or similar antibody-like fragments, leucine zipper motifs, a coiled coil domain, a hydrophobic region, a hydrophilic region, a polypeptide comprising a free thiol which forms an intermolecular disulfide bond between two or more multimerization domains, a “protuberance-into-cavity” domain, beta-lactoglobulin, or fragments thereof.

The molecules of interest may include a half-life extending moiety. The term “half-life extending moiety” refers to a pharmaceutically acceptable moiety, domain, or “vehicle” covalently linked or conjugated to the subject compound, that prevents or mitigates activity-diminishing chemical modification of the subject compound, increases half-life or other pharmacokinetic properties (e.g., rate of absorption), reduces toxicity, improves solubility, increases biological activity and/or target selectivity of the subject compound with respect to a target of interest, increases manufacturability, and/or reduces immunogenicity of the subject compound, compared to an unconjugated form of the subject compound.

In certain embodiments, the half-life extending moiety is a polypeptide that binds a serum protein, such as an immunoglobulin (e.g., IgG) or a serum albumin (e.g., human serum albumin (HSA)). Polyethylene glycol is an example of a useful half-life extending moiety. Exemplary half-life extending moieties include a polyalkylene glycol moiety (e.g., PEG), a serum albumin or a fragment thereof, a transferrin receptor or a transferrin-binding portion thereof, and a moiety comprising a binding site for a polypeptide that enhances half-life in vivo, a copolymer of ethylene glycol, a copolymer of propylene glycol, a carboxymethylcellulose, a polyvinyl pyrrolidone, a poly-1,3-dioxolane, a poly-1,3,6-trioxane, an ethylene/maleic anhydride copolymer, a polyaminoacid (e.g., polylysine), a dextran n-vinyl pyrrolidone, a poly n-vinyl pyrrolidone, a propylene glycol homopolymer, a propylene oxide polymer, an ethylene oxide polymer, a polyoxyethylated polyol, a polyvinyl alcohol, a linear or branched glycosylated chain, a polysialic acid, a polyacetal, a long chain fatty acid, a long chain hydrophobic aliphatic group, an immunoglobulin Fc domain (see, e.g., U.S. Pat. No. 6,660,843), an albumin (e.g., human serum albumin; see, e.g., U.S. Pat. No. 6,926,898 and US 2005/0054051; U.S. Pat. No. 6,887,470), a transthyretin (TTR; see, e.g., US 2003/0195154; 2003/0191056), or a thyroxine-binding globulin (TBG).

An extended half-life can also be achieved via a controlled or sustained release dosage form of the subject compounds, e.g., as described by Gilbert S. Banker and Christopher T. Rhodes, Sustained and controlled release drug delivery system. In Modern Pharmaceutics, Fourth Edition, Revised and Expanded, Marcel Dekker, New York, 2002, 11. This can be achieved through a variety of formulations, including liposomes and drug-polymer conjugates.

In certain embodiments, the half-life extending moiety is a fatty acid. Any convenient fatty acids may be used in the subject modified compounds. See e.g., Chae et al., “The fatty acid conjugated exendin-4 analogs for type 2 antidiabetic therapeutics”, J. Control Release. 2010 May 21; 144(1):10-6.

In certain embodiments, the compound is modified to include a specific binding moiety. The specific binding moiety is a moiety that is capable of specifically binding to a second moiety that is complementary to it. In some embodiments, the specific binding moiety binds to the complementary second moiety with an affinity of at least 10⁻⁷ M (e.g., as measured by a K_{D} of 100 nM or less, such as 30 nM or less, I0 nM or less, 3 nM or less, 1 nM or less, 300 μM or less, or 100 μM or even less). Complementary binding moiety pairs of specific binding moieties include, but are not limited to, a ligand and a receptor, an antibody and an antigen, complementary polynucleotides, complementary protein homo- or heterodimers, an aptamer and a small molecule, a polyhistidine tag and nickel, and a chemoselective reactive group (e.g., a thiol) and an electrophilic group (e.g., with which the reactive thiol group can undergo a Michael addition). The specific binding pairs may include analogs, derivatives and fragments of the original specific binding member. For example, an antibody directed to a protein antigen may also recognize peptide fragments, chemically synthesized, labeled protein, derivatized protein, etc. so long as an epitope is present. Protein domains of interest that find use as specific binding moieties include, but are not limited to, Fc domains, or similar antibody-like fragments, leucine zipper motifs, a coiled coil domain, a hydrophobic region, a hydrophilic region, a polypeptide comprising a free thiol which forms an intermolecular disulfide bond between two or more multimerization domains, or a “protuberance-into-cavity” domain (see e.g., WO 94/10308; U.S. Pat. No. 5,731,168, Lovejoy et al. (1993), Science 259: 1288-1293; Harbury et al. (1993), Science 262: 1401-05; Harbury et al. (1994), Nature 371:80-83; Hakansson et al. (1999), Structure 7: 255-64.

In certain embodiments, the molecule of interest is a linked specific binding moiety that specifically binds a target protein. The linked specific binding moiety can be an antibody, an antibody fragment, an aptamer or a second D-peptidic binding domain. The linked specific binding moiety can specifically bind any convenient target protein, e.g., a target protein that is desirable to target in conjunction with PD-1 in the subject methods of treatment. Target proteins of interest include, but are not limited to, PDGF (e.g., PDGF-B), VEGF-A, VEGF-B, VEGF-C, VEGF-D, EGF, EGFR, Her2, PD-L1, OX-40 and LAG3. In certain instances, the linked specific binding moiety is a second D-peptidic binding domain that targets PDGF-B.

In certain embodiments, the specific binding moiety is an affinity tag such as a biotin moiety. Exemplary biotin moieties include biotin, desthiobiotin, oxybiotin, 2′-iminobiotin, diaminobiotin, biotin sulfoxide, biocytin, etc. In some embodiments, the biotin moiety is capable of specifically binding with high affinity to a chromatography support that contains immobilized avidin, neutravidin or streptavidin. Biotin moieties can bind to streptavidin with an affinity of at least 10⁻⁸M. In some embodiments, a monomeric avidin support may be used to specifically bind a biotin-containing compound with moderate affinity thereby allowing bound compounds to be later eluted competitively from the support (e.g., with a 2 mM biotin solution) after non-biotinylated polypeptides have been washed away. In certain instances, the biotin moiety is capable of binding to an avidin, neutravidin or streptavidin in solution to form a multimeric compound, e.g., a dimeric, or tetrameric complex of D-peptidic compounds with the avidin, neutravidin or streptavidin. A biotin moiety may also include a linker, e.g., -LC-biotin, -LC-LC-Biotin, -SLC-Biotin or -PEGₙ-Biotin where n is 3-12 (commercially available from Pierce Biotechnology).

In certain embodiments, the compound is modified to include a detectable label. Examples of detectable labels include labels that permit both the direct and indirect measurement of the presence of the subject D-peptidic compound. Examples of labels that permit direct measurement of the compound include radiolabels, fluorophores, dyes, beads, nanoparticles (e.g., quantum dots), chemiluminescers, colloidal particles, paramagnetic labels and the like. Radiolabels may include radioisotopes, such as ³⁵S, ¹⁴C, ¹²⁵I, ³H, ⁶⁴Cu and ¹³¹I. The subject compounds can be labeled with the radioisotope using any convenient techniques, such as those described in Current Protocols in Immunology, Volumes 1 and 2, Coligen et al., Ed. Wiley-Interscience, New York, N.Y., Pubs. (1991), and radioactivity can be measured using scintillation counting or positron emission. Examples of detectable labels which permit indirect measurement of the presence of the modified compound include enzymes where a substrate may provide for a colored or fluorescent product. For example, the compound may include a covalently bound enzyme capable of providing a detectable product signal after addition of suitable substrate. Instead of covalently binding the enzyme to the compound, the compound may include a first member of specific binding pair which specifically binds with a second member of the specific binding pair that is conjugated to the enzyme, e.g. the compound may be covalently bound to biotin and the enzyme conjugate to streptavidin. Examples of suitable enzymes for use in conjugates include horseradish peroxidase, alkaline phosphatase, malate dehydrogenase and the like. Where not commercially available, such enzyme conjugates may be readily produced by any convenient techniques.

In certain embodiments, the detectable label is a fluorophore. The term “fluorophore” refers to a molecule that, when excited with light having a selected wavelength, emits light of a different wavelength, which may emit light immediately or with a delay after excitation. Fluorophores, include, without limitation, fluorescein dyes, e.g., 5-carboxyfluorescein (5-FAM), 6-carboxyfluorescein (6-FAM), 2′,4′,1,4-tetrachlorofluorescein (TET), 2′,4′,5′,7′,1,4-hexachlorofluorescein (HEX), and 2′,7′-dimethoxy-4′,5′-dichloro-6-carboxyfluorescein (JOE); cyanine dyes, e.g. Cy3, CY5, Cy5.5, QUASAR™ dyes etc.; dansyl derivatives; rhodamine dyes e. g. 6-carboxytetramethylrhodamine (TAMRA), CAL FLUOR dyes, tetrapropano-6-carboxyrhodamine (ROX). BODIPY fluorophores, ALEXA dyes, Oregon Green, pyrene, perylene, benzopyrene, squarine dyes, coumarin dyes, luminescent transition metal and lanthanide complexes and the like. The term fluorophore includes excimers and exciplexes of such dyes.

In some embodiments, the compound includes a detectable label, such as a radiolabel. In certain embodiments, the radiolabel suitable for use in PET, SPECT and/or MR imaging. In certain embodiments, the radiolabel is a PET imaging label. In certain cases, the compound is radiolabeled with ¹⁸F, ⁶⁴Cu, ⁶⁸Ga, ¹¹¹In, ⁹⁹mTc or ⁸⁶Y.

The detectable label may be attached to the D-peptidic compound at any convenient position and via any convenient chemistry. Methods and materials of interest include, but are not limited to those described by U.S. Pat. No. 8,545,809; Meares et al., 1984, Ace Chem Res 17:202-209; Scheinberg et al., 1982, Science 215:1511-13; Miller et al., 2008, Angew Chem Int Ed 47:8998-9033; Shirrmacher et al., 2007, Bioconj Chem 18:2085-89; Hohne et al., 2008, Bioconj Chem 19:1871-79; Ting et al., 2008, Fluorine Chem 129:349-58, the labeling method of Poethko et al. (J. Nucl. Med. 2004; 45: 892-902) in which 4-[18F]fluorobenzaldehyde is first synthesized and purified (Wilson et al, J. Labeled Compounds and Radiopharm. 1990; XXVIII: 1189-1199) and then conjugated to a peptide, labeling with succinimidyl [18F]fluorobenzoate (SFB) (e.g., Vaidyanathan et al., 1992, Int. J. Rad. Appl. Instrum. B 19:275), other acyl compounds (Tada et al., 1989, Labeled Compd. Radiopharm. XXVII:1317; Wester et al., 1996, Nucl. Med. Biol. 23:365; Guhlke et al., 1994, Nucl. Med. Biol 21:819), or click chemistry adducts (Li et al., 2007, Bioconj Chem. 18:1987).

Any convenient synthetic methods or bioconjugation methods may be utilized in preparing the subject modified D-peptidic domains and compounds. In certain cases, the detectable label is connected to the compound via an optional linker. In certain embodiments, the detectable label is connected to the N-terminal of a domain or the compound. In certain embodiments, the detectable label is connected to the C-terminal of a domain or the compound. In certain embodiments, the detectable label is connected to a non-terminal residue of a domain or the compound, e.g., via a side chain moiety. In certain embodiments, the detectable label is connected to the N-terminal D-peptidic extension moiety of a domain or the compound via an optional linker. In some embodiments, the N-terminal D-peptidic extension moiety is modified to include a reactive functional group which is capable of reacting with a compatible functional group of a radiolabel containing moiety. Any convenient reactive functional groups, chemistries and radiolabel containing moieties may be utilized to attach a detectable label to the compound, including but not limited to, click chemistry, an azide, an alkyne, a cyclooctyne, copper-free click chemistry, a nitrone, a chelating group (e.g., selected from DOTA, TETA, NOTA, NODA, (tert-Butyl)₂NODA, NETA, C-NETA, L-NETA, S-NETA, NODA-MPAA, and NODA-MPAEM), a propargyl-glycine residue, etc.

In certain instances, the molecule of interest is a second active agent, e.g., an active agent or drug that finds use in conjunction with targeting the target protein in the subject methods of treatment. In certain instances, the molecule of interest is a small molecule, a chemotherapeutic, an antibody, an antibody fragment, an aptamer, or a L-protein. In some embodiments, the compound is modified to include a moiety that is useful as a pharmaceutical (e.g., a protein, nucleic acid, organic small molecule, etc.). Exemplary pharmaceutical proteins include, e.g., cytokines, antibodies, chemokines, growth factors, interleukins, cell-surface proteins, extracellular domains, cell surface receptors, cytotoxins, etc. Exemplary small molecule pharmaceuticals include small molecule cytotoxins or therapeutic agents. Any convenient therapeutic or diagnostic agent (e.g., as described herein) can be conjugated to a D-peptidic compound. A variety of therapeutic agents including, but not limited to, anti-cancer agents, antiproliferative agents, cytotoxic agents and chemotherapeutic agents are described below in the section entitled Combination Therapies, any one of which can be adapted for use in the subject modified compounds. Exemplary chemotherapeutic agents of interest include, for example, Gemcitabine, Docetaxel, Bleomycin, Erlotinib, Gefitinib, Lapatinib, Imatinib, Dasatinib, Nilotinib, Bosutinib, Crizotinib, Ceritinib, Trametinib, Bevacizumab, Sunitinib, Sorafenib, Trastuzumab, Ado-trastuzumab emtansine, Rituximab, Ipilimumab, Rapamycin, Temsirolimus, Everolimus, Methotrexate, Doxorubicin, Abraxane, Folfirinox, Cisplatin, Carboplatin, 5-fluorouracil, Teysumo, Paclitaxel, Prednisone, Levothyroxine, Pemetrexed, navitoclax, ABT-199, nivolumab. Any exemplary cytotoxic agents that find use in ADC can be adapted for use in the subject modified D-peptidic compounds. Cytotoic agents of interest include, but are not limited to, auristatins (e.g., MMAE, MMAF), maytansines, dolastatins, calicheamicins, duocarmycins, pyrrolobenzodiazepines (PBDs), centanamycin (ML-970; indolecarboxamide), doxorubicin, a-Amanitin, and derivatives and analogs thereofIn certain embodiments, the compound may include a cell penetrating peptide (e.g., tat). The cell penetrating peptide may facilitate cellular uptake of the molecule. Any convenient tag polypeptides and their respective antibodies may be used. Examples include poly-histidine (poly-his) or poly-histidine-glycine (poly-his-gly) tags; the flu HA tag polypeptide and its antibody 12CA5 [Field et al., Mol. Cell. Biol. 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology 6:1204-1210 (1988)]; the KT3 epitope peptide [Martin et al., Science 255:192-194 (1992)]; tubulin epitope peptide [Skinner et al., J. Biol. Chem. 266:15163-15166 (1991)]; and the T7 gene 10 protein peptide tag [Lutz-Freyermuth et al., Proc. Natl. Acad. Sci. U.S.A. 87:6393-6397 (1990)].

The molecules of interest may be attached to the subject modified compounds via any convenient method. In some embodiments, a molecule of interest is attached via covalent conjugation to a terminal amino acid residue, e.g., at the amino terminus or at the carboxylic acid terminus. The molecule of interest may be attached to the D-peptidic domain via a single bond or a suitable linker, e.g., a PEG linker, a peptidic linker including one or more amino acids, or a saturated hydrocarbon linker. A variety of linkers (e.g., as described herein) find use in the subject modified compounds. Any convenient reagents and methods may be used to include a molecule of interest in a subject domains, for example, conjugation methods as described in G. T. Hermanson, “Bioconjugate Techniques” Academic Press, 2nd Ed., 2008, solid phase peptide synthesis methods, or fusion protein expression methods. Functional groups that may be used in covalently bonding the domain, via an optional linker, to produce the modified compound include: hydroxyl, sulfhydryl, amino, and the like. Certain moieties on the molecules of interest and/or GA domain motif may be protected using convenient blocking groups, see, e.g. Green & Wuts, Protective Groups in Organic Synthesis (John Wiley & Sons) 3rd Ed. (1999). The particular molecule of interest and site of attachment to the domain may be chosen so as not to substantially adversely interfere with the desired binding activity for the target protein.

The molecule of interest may be peptidic. It is understood that a molecule of interest may further include one or more non-peptidic groups including, but not limited to, a biotin moiety and/or a linker. Any convenient protein domains may be adapted and utilized as molecules of interest in the subject modified peptidic compounds. Protein domains of interest include, but are not limited to, any convenient serum protein, serum albumin (e.g., human serum albumin; see, e.g., U.S. Pat. No. 6,926,898 and US 2005/0054051; U.S. Pat. No. 6,887,470), a transferrin receptor or a transferrin-binding portion thereof, immunoglobulin (e.g., IgG), an immunoglobulin Fc domain (see, e.g., U.S. Pat. No. 6,660,843), a transthyretin (TTR; see, e.g., US 2003/0195154; 2003/0191056), a thyroxine-binding globulin (TBG), or a fragment thereof.

A multimerizing group is any convenient group that is capable of forming a multimer (e.g., a dimer, a trimer, or a dendrimer), e.g., by mediating binding between two or more compounds (e.g., directly or indirectly via a multivalent binding moiety), or by connecting two or more compounds via a covalent linkage. In some embodiments, the multimerizing group Z is a chemoselective reactive functional group that conjugates to a compatible function group on a second D-peptidic compound. In other cases, the multimerizing group is a specific binding moiety (e.g., biotin or a peptide tag) that specifically binds to a multivalent binding moiety (e.g., a streptavidin or an antibody). In some embodiments, the compound includes a multimerizing group and is a monomer that has not yet been multimerized.

Chemoselective reactive functional groups for inclusion in the subject D-peptidic compounds, include, but are not limited to: an azido group, an alkynyl group, a phosphine group, a cysteine residue, a C-terminal thioester, aryl azides, maleimides, carbodiimides, N-hydroxysuccinimide (NHS)-esters, hydrazides, PFP-esters, hydroxymethyl phosphines, psoralens, imidoesters, pyridyl disulfides, isocyanates, aminooxy-, aldehyde, keto, chloroacetyl, bromoacetyl, and vinyl sulfones.

### Polynucleotides

Also provided are polynucleotides that encode a sequence corresponding to the subject peptidic compounds as described herein. The polynucleotide can encode a L-peptidic compound that specifically binds to a D-target protein.

In some embodiments, the polynucleotide encodes a peptidic compound that includes between 25 and 80 residues, between 30 and 80 residues, between 30 and 70 residues, between 40 and 70 residues, between 45 and 60 residues, between 45 and 60 residues, or between 45 and 55 residues. In certain instances, the polynucleotide encodes a peptidic compound sequence of between 35 and 55 residues, such as between 40 and 55 residues, or between 45 and 55 residues.

In certain embodiments, the polynucleotide encodes a peptidic compound sequence of 45, 46, 47, 48, 49, 50, 51, 52 or 53 residues.

In certain embodiments, the polynucleotide is a replicable expression vector that includes a nucleic acid sequence encoding a L-peptidic compound that may be expressed in a protein expression system. In certain embodiments, the polynucleotide is a replicable expression vector that includes a nucleic acid sequence encoding a gene fusion, where the gene fusion encodes a fusion protein including the L-peptidic compound fused to all or a portion of a viral coat protein.

In certain embodiments, the subject polynucleotides are capable of being expressed and displayed in a cell-based or cell-free display system. Any convenient display methods may be used to display L-peptidic compounds encoded by the subject polynucleotides, such as cell-based display techniques and cell-free display techniques. In certain embodiments, cell-based display techniques include phage display, bacterial display, yeast display and mammalian cell display. In certain embodiments, cell-free display techniques include mRNA display and ribosome display.

### Methods

### PD-1 Methods

Aspects of this disclosure include D-peptidic compounds that specifically bind to programmed cell death protein 1 (PD-1) and methods of using same. The herein-described compounds may be employed in a variety of methods. One such method includes contacting a subject compound with a PD-1 target protein under conditions suitable for binding of PD-1 to produce a complex. In some embodiments, the method includes administering a D-peptidic compound to a subject, where the compound binds to PD-1 in the subject.

The PD-1 specific D-peptidic compounds find use in the treatment of a cancer or for inhibiting tumor growth or progression in a subject in need thereof. In some embodiments, the cancer is, for example without limitation, gastric cancer, sarcoma, lymphoma, Hodgkin's lymphoma, leukemia, head and neck cancer, thymic cancer, epithelial cancer, salivary cancer, liver cancer, stomach cancer, thyroid cancer, lung cancer (including, for example, non-small-cell lung carcinoma), ovarian cancer, breast cancer, prostate cancer, esophageal cancer, pancreatic cancer, glioma, leukemia, multiple myeloma, renal cell carcinoma, bladder cancer, cervical cancer, chonocarcinoma, colon cancer, oral cancer, skin cancer, and melanoma.

In another aspect, the present disclosure provides a method for enhancing the immune response or therapeutic effect of a drug or agent for the treatment of a cancer in a mammal, particularly a human, e.g., by activating T cells. In some embodiments, the subject compounds are capable of negatively regulating PD-1-associated immune responses. In particular embodiments, PD-1 specific D-peptidic compounds are used to treat or prevent immune disorders by virtue of increasing or reducing the T cell response, e.g., mediated by TcR/CD28. Disorders susceptible to treatment with compositions of the invention include but are not limited to rheumatoid arthritis, multiple sclerosis, inflammatory bowel disease, Crohn's disease, systemic lupus erythematosis, type I diabetes, transplant rejection, graft-versus-host disease, hyperproliferative immune disorders, cancer, and infectious diseases.

A subject compound may inhibit at least one activity of its PD-1 target in the range of 10% to 100%, e.g., by 10% or more, 20% or more, 30% or more, 40% or more, 50% or more, 60% or more, 70% or more, 80% or more, or 90% or more. In certain assays, a subject compound may inhibit its PD-1 target with an IC₅₀ of 1×10⁻⁵ M or less (e.g., 1×10⁶ M or less, 1×10⁻⁷ M or less, 1×10⁻⁸ M or less, 1×10⁻⁹ M or less, 1×10⁻¹⁰ M or less, or 1×10⁻¹¹ M or less). In certain assays, a subject compound may inhibit its PD-1 target with an IC₂₀ of 1×10⁶ M or less (e.g., 500 nM or less, 200 nM or less, 100 nM or less, 30 nM or less, 10 nM or less, 3 nM or less, or nM or less). In certain assays, a subject compound may inhibit its PD-1 target with an IC₁₀ of 1×10⁻⁶ M or less (e.g., 500 nM or less, 200 nM or less, 100 nM or less, 30 nM or less, 10 nM or less, 3 nM or less, or 1 nM or less). In assays in which a mouse is employed, a subject compound may have an ED₅₀ of less than 1 μg/mouse (e.g., 1 ng/mouse to about 1 μg/mouse).

In some embodiments, the subject method is an in vitro method that includes contacting a sample with a subject compound that specifically binds with high affinity to a target molecule. In certain embodiments, the sample is suspected of containing the target molecule and the subject method further includes evaluating whether the compound specifically binds to the target molecule. In certain embodiments, the target molecule is a naturally occurring L-protein and the compound is D-peptidic. In certain embodiments, the subject compound is a modified compound that includes a label, e.g., a fluorescent label, and the subject method further includes detecting the label, if present, in the sample, e.g., using optical detection. In certain embodiments, the compound is modified with a support, such that any sample that does not bind to the compound may be removed (e.g., by washing). The specifically bound target protein, if present, may then be detected using any convenient means, such as, using the binding of a labeled target specific probe or using a fluorescent protein reactive reagent. In another embodiment of the subject method, the sample is known to contain the target protein. In certain embodiments, the target PD-1 protein is a synthetic D-protein and the compound is L-peptidic. In certain embodiments, the target PD-1 protein is a L-protein and the compound is D-peptidic.

In certain embodiments, a subject compound may be contacted with a cell in the presence of PD-1, and a PD-1 response phenotype of the cell monitored. Exemplary PD-1 assays include assays using isolated protein in cell free systems, in vitro using cultured cells or in vivo assays. Exemplary PD-1 assays include, but are not limited to a receptor tyrosine kinase inhibition assay (see, e.g., Cancer Research Jun. 15, 2006; 66:6025-6032), an in vitro HUVEC proliferation assay (FASEB Journal 2006; 20: 2027-2035; Wells et al., Biochemistry 1998, 37, 17754-17764), an in vivo solid tumor disease assay (U.S. Pat. No. 6,811,779) and an in vivo angiogenesis assay (FASEB Journal 2006; 20: 2027-2035). The descriptions of these assays are hereby incorporated by reference. The protocols that may be employed in these methods are numerous and include, but are not limited to cell-free assays, e.g., binding assays; cellular assays in which a cellular phenotype is measured, e.g., gene expression assays; and in vivo assays that involve a particular animal (which, in certain embodiments may be an animal model for a condition related to the target).

In some embodiments, the subject method is in vivo and includes administering to a subject a D-peptidic compound that specifically binds with high affinity to a target molecule. In certain embodiments, the compound is administered as a pharmaceutical preparation. A variety of subjects are treatable according to the subject methods. Generally such subjects are “mammals” or “mammalian,” where these terms are used broadly to describe organisms which are within the class mammalia, including the orders carnivore (e.g., dogs and cats), rodentia (e.g., mice, guinea pigs and rats), and primates (e.g., humans, chimpanzees and monkeys). In some embodiments, the subject is human. The subject can be a subject in need of prevention of treatment of a disease or condition associated with angiogenesis in a subject (e.g., as described herein).

The term “treating” or “treatment” as used herein means the treating or treatment of a disease or medical condition in a patient, such as a mammal (such as a human) that includes: (a) preventing the disease or medical condition from occurring, such as, prophylactic treatment of a subject; (b) ameliorating the disease or medical condition, such as, eliminating or causing regression of the disease or medical condition in a patient; (c) suppressing the disease or medical condition, for example by, slowing or arresting the development of the disease or medical condition in a patient; or (d) alleviating a symptom of the disease or medical condition in a patient. As such, treatment also includes situations where the pathological condition, or at least symptoms associated therewith, are completely inhibited, e.g., prevented from happening, or stopped, e.g., terminated, such that the subject no longer suffers from the pathological condition, or at least the symptoms that characterize the pathological condition. Treatment may also manifest in the form of a modulation of a surrogate marker of the disease condition, e.g., as described above.

In certain embodiments, the subject methods include administering a compound, such as a PD-1 binding compound, and then detecting the compound after it has bound to its target protein. In some methods, the same compound can serve as both a therapeutic and a diagnostic compound. The PD-1 binding compounds of the present disclosure are therapeutically useful for treating any disease or condition which is improved, ameliorated, inhibited or prevented by removal, inhibition, or reduction of a PD-1 protein, or a fragment thereof.

In some embodiments, the subject method is a method of treating a subject suffering from a disease condition, the method including administering to the subject an effective amount of a subject compound that specifically binds with high affinity to a PD-1 protein so that the subject is treated for the disease condition.

In some embodiments, the subject method is a method of inhibiting tumor growth in a subject, the method comprising administering to a subject an effective amount of a subject compound that specifically binds with high affinity to the PD-1 protein. In certain embodiments, the tumor is a solid tumor. In certain embodiments, the tumor is a non-solid tumor.

The amount of compound administered can be determined using any convenient methods to be an amount sufficient to produce the desired effect in association with a pharmaceutically acceptable diluent, carrier or vehicle. The specifications for the unit dosage forms of the present disclosure will depend on the particular compound employed and the effect to be achieved, and the pharmacodynamics associated with each compound in the subject.

In some embodiments, a single dose of the subject compound is administered. In other embodiments, multiple doses of the subject compound are administered. Where multiple doses are administered over a period of time, the D-peptidic compound is administered twice daily (qid), daily (qd), every other day (qod), every third day, three times per week (tiw), or twice per week (biw) over a period of time. For example, a compound is administered qid, qd, qod, tiw, or biw over a period of from one day to about 2 years or more. For example, a compound is administered at any of the aforementioned frequencies for one week, two weeks, one month, two months, six months, one year, or two years, or more, depending on various factors.

Any of a variety of methods can be used to determine whether a treatment method is effective. For example, a biological sample obtained from an individual who has been treated with a subject method can be assayed for the presence and/or extent of angiogenesis. Assessment of the effectiveness of the methods of treatment on the subject can include assessment of the subject before, during and/or after treatment, using any convenient methods. Aspects of the subject methods further include a step of assessing the therapeutic response of the subject to the treatment.

In some embodiments, the method includes assessing the condition of the subject, including diagnosing or assessing one or more symptoms of the subject which are associated with the disease or condition of interest being treated (e.g., as described herein). In some embodiments, the method includes obtaining a biological sample from the subject and assaying the sample, e.g., for the presence of angiogenesis that is associated with the disease or condition of interest (e.g., as described herein). The sample can be a cellular sample. In some embodiments, the sample is a biopsy. The assessment step(s) of the subject method can be performed at one or more times before, during and/or after administration of the subject compounds, using any convenient methods.

In some embodiments, a subject compound or a salt thereof, e.g., as defined herein, finds use in medicine, particularly in the in vivo diagnosis or imaging, for example by PET, of a disease or condition associated with angiogenesis or cancer. In certain embodiments, the compound is a modified compound that includes a detectable label, and the method further includes detecting the label in the subject. The selection of the label depends on the means of detection. Any convenient labeling and detection systems may be used in the subject methods, see e.g., Baker, “The whole picture,” Nature, 463, 2010, p977-980. In certain embodiments, the compound includes a fluorescent label suitable for optical detection. In certain embodiments, the compound includes a radiolabel for detection using positron emission tomography (PET) or single photon emission computed tomography (SPECT). In some embodiments, the compound includes a paramagnetic label suitable for tomographic detection. The subject compound may be labeled, as described above, although in some methods, the compound is unlabeled and a secondary labeling agent is used for imaging. In certain embodiments, the subject methods include diagnosis of a disease condition in a subject by comparing the number, size, and/or intensity of labeled loci, to corresponding baseline values. The base line values can represent the mean levels in a population of undiseased subjects, or previous levels determined in the same subject.

In some embodiments, radiolabeled compounds may be administered to subjects for PET imaging in amounts sufficient to yield the desired signal. In certain instances, the radionuclide dosage is of 0.01 to 100 mCi, such as 0.1 to 50 mCi, or 1 to 20 mCi, which is sufficient per 70 kg bodyweight. The radiolabeled compounds may therefore be formulated for administration using any convenient physiologically acceptable carriers or excipients. For example, the compounds, optionally with the addition of pharmaceutically acceptable excipients, may be suspended or dissolved in an aqueous medium, with the resulting solution or suspension then being sterilized. Also provided is the use of a radiolabeled compound or a salt thereof as described herein for the manufacture of a radiopharmaceutical for use in a method of in vivo imaging, e.g., PET imaging, such as imaging of a disease or condition associated with angiogenesis; involving administration of the radiopharmaceutical to a human or animal body and generation of an image of at least part of said body.

In some embodiments, the method is a method of monitoring the effect of treatment of a human or animal body with a drug, e.g., a cytotoxic agent, to combat a condition associated with angiogenesis e.g., cancer, said method including administering to said body a radiolabelled compound or a salt thereof and detecting the uptake of the compound by cell receptors, such as endothelial cell receptors, e.g., alpha.v.beta.3 receptors, the administration and detection optionally being effected repeatedly, e.g. before, during and after treatment with said drug.

In some embodiments, the method is a method for in vivo diagnosis or imaging of a disease or condition associated with angiogenesis including administering to a subject a D-peptidic compound and imaging at least a part of the subject. In certain embodiments, the imaging comprises PET imaging and the administering comprises administering the compound to the vascular system of the subject. In some instances, the method further includes detecting uptake of the compound by cell receptors. In certain instances, the target is PD-1 and the subject is human. In certain embodiments, the method includes administering a therapeutic antibody, e.g., bevacizumab (Avastin) or nivolumab, to the subject, wherein the disease or condition is a condition associated with cancer.

The subject methods may be diagnostic methods for detecting the expression of a target protein in specific cells, tissues, or serum, in vitro or in vivo. In some embodiments, the subject method is a method for in vivo imaging of a target protein in a subject. The methods may include administering the compound to a subject presenting with symptoms of a disease condition related to a target protein. In some embodiments, the subject is asymptomatic. The subject methods may further include monitoring disease progression and/or response to treatment in subjects who have been previously diagnosed with the disease.

The subject PD-1 binding compounds may be used as affinity purification agents. In this process, the compounds are immobilized on a solid phase such a Sephadex resin or filter paper, using any convenient methods. The subject PD-1 binding compound is contacted with a sample containing the PD-1 protein (or fragment thereof) to be purified, and thereafter the support is washed with a suitable solvent that will remove substantially all the material in the sample except the PD-1 protein, which is bound to the immobilized compound. Finally, the support is washed with another suitable solvent, such as glycine buffer, pH 5.0 that will release the PD-1 protein from the immobilized compound.

The subject PD-1 binding compounds may also be useful in diagnostic assays for PD-1 protein, e.g., detecting its expression in specific cells, tissues, or serum. Such diagnostic methods may be useful in cancer diagnosis. For diagnostic applications, the subject compound may be modified as described above.

### Combination Therapies

In some embodiments, the subject compounds may be administered in combination with one or more additional active agents or therapies. Any convenient agents may be utilized, including compounds useful for treating diseases that are targeted by the subject methods. The terms “agent,” “compound,” and “drug” are used interchangeably herein. Additional active agents or therapies include, but are not limited to, a small molecule, an antibody, an antibody fragment, an aptamer, a L-protein, a second target-binding molecule such as a second D-peptidic compound, a chemotherapeutic agent, surgery, catheter devices, and radiation. Combination therapy includes administration of a single pharmaceutical dosage formulation which contains the subject compound and one or more additional agents; as well as administration of the subject compound and one or more additional agent(s) in its own separate pharmaceutical dosage formulation. For example, a subject compound and a cytotoxic agent, a chemotherapeutic agent or a growth inhibitory agent can be administered to the patient together in a single dosage composition such as a combined formulation, or each agent can be administered in a separate dosage formulation. Where separate dosage formulations are used, the subject compound and one or more additional agents can be administered concurrently, or at separately staggered times, e.g., sequentially.

The terms “co-administration” and “in combination with” include the administration of two or more therapeutic agents (e.g., a D-peptidic compound and a second agent) either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent (e.g., a D-peptidic compound) can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

“Concomitant administration” of a known therapeutic drug with a pharmaceutical composition of the present disclosure means administration of the D-peptidic compound and second agent at such time that both the known drug and the composition of the present disclosure will have a therapeutic effect. Such concomitant administration may involve concurrent (i.e. at the same time), prior, or subsequent administration of the drug with respect to the administration of a subject D-peptidic compound. Routes of administration of the two agents may vary, where representative routes of administration are described in greater detail below. A person of ordinary skill in the art would have no difficulty determining the appropriate timing, sequence and dosages of administration for particular drugs and compounds of the present disclosure.

In some embodiments, the compounds (e.g., a subject D-peptidic compound and a second agent) are administered to the subject within twenty-four hours of each other, such as within 12 hours of each other, within 6 hours of each other, within 3 hours of each other, or within 1 hour of each other. In certain embodiments, the compounds are administered within 1 hour of each other. In certain embodiments, the compounds are administered substantially simultaneously. By administered substantially simultaneously is meant that the compounds are administered to the subject within about 10 minutes or less of each other, such as 5 minutes or less, or 1 minute or less of each other.

Also provided are pharmaceutical preparations of the subject compounds and the second active agent. In pharmaceutical dosage forms, the compounds may be administered in the form of their pharmaceutically acceptable salts, or they may also be used alone or in appropriate association, as well as in combination, with other pharmaceutically active compounds.

Dosage levels of the order of from about 0.01 mg to about 140 mg/kg of body weight per day are useful in representative embodiments, or alternatively about 0.5 mg to about 7 g per patient per day. Those of skill will readily appreciate that dose levels can vary as a function of the specific compound, the severity of the symptoms and the susceptibility of the subject to side effects. Dosages for a given compound are readily determinable by those of skill in the art by a variety of means.

The amount of active ingredient that may be combined with the carrier materials to produce a single dosage form will vary depending upon the host treated and the particular mode of administration. For example, a formulation intended for the oral administration of humans may contain from 0.5 mg to 5 g of active agent compounded with an appropriate and convenient amount of carrier material which may vary from about 5 to about 95 percent of the total composition. Dosage unit forms will generally contain between from about 1 mg to about 500 mg of an active ingredient, such as 25 mg, 50 mg, 100 mg, 200 mg, 300 mg, 400 mg, 500 mg, 600 mg, 800 mg, or 1000 mg.

It will be understood, however, that the specific dose level for any particular patient will depend upon a variety of factors including the age, body weight, general health, sex, diet, time of administration, route of administration, rate of excretion, drug combination and the severity of the particular disease undergoing therapy.

Any convenient second agents can find use in the subject methods. In some embodiments, the second active agent specifically binds a target protein selected from platelet-derived growth factor (PDGF), VEGF-A, VEGF-B, VEGF-C, VEGF-D, EGF, EGFR, Her2, PD-L1, OX-40, LAG3, Ang2, IL-1, IL-6 and IL-17. Second active agents of interest include, but are not limited to, pegpleranib (Fovista), ranibizumab (Lucentis), trastuzumab (Herceptin), bevacizumab (Avastin), aflibercept (Eylea), nivolumab (Opdivo), atezolizumab, durvalumab, gefitinib, erlotinib and pembrolizumab (Keytruda).

For the treatment of cancer, the subject compounds can be administered in combination with a chemotherapeutic agent selected from the group consisting of taxanes, nucleoside analogs, steroids, anthracyclines, thyroid hormone replacement drugs, thymidylate-targeted drugs, Chimeric Antigen Receptor/T cell therapies, Chimeric Antigen Receptor/NK cell therapies, apoptosis regulator inhibitors (e.g., B cell CLL/lymphoma 2 (BCL-2) BCL-2-like 1 (BCL-XL) inhibitors), CARP-1/CCARI (Cell division cycle and apoptosis regulator 1) inhibitors, colony-stimulating factor-1 receptor (CSF1R) inhibitors, CD47 inhibitors, cancer vaccine (e.g., a Th17-inducing dendritic cell vaccine) and other cell therapies. Specific chemotherapeutic agents include, for example, Gemcitabine, Docetaxel, Bleomycin, Erlotinib, Gefitinib, Lapatinib, Imatinib, Dasatinib, Nilotinib, Bosutinib, Crizotinib, Ceritinib, Trametinib, Bevacizumab, nivolumab, Sunitinib, Sorafenib, Trastuzumab, Ado-trastuzumab emtansine, Rituximab, Ipilimumab, Rapamycin, Temsirolimus, Everolimus, Methotrexate, Doxorubicin, Abraxane, Folfirinox, Cisplatin, Carboplatin, 5-fluorouracil, Teysumo, Paclitaxel, Prednisone, Levothyroxine, Pemetrexed, navitoclax, ABT-199.

For the treatment of cancer (e.g., melanoma, non-small cell lung cancer or a lymphoma such as Hodgkin's lymphoma), the subject compounds can be administered in combination with an immune checkpoint inhibitor. Any convenient checkpoint inhibitors can be utilized, including but not limited to, cytotoxic T-lymphocyte-associated antigen 4 (CTLA-4) inhibitors, and programmed death ligand 1 PD-L1 inhibitors. Exemplary checkpoint inhibitors of interest include, but are not limited to, ipilimumab, pembrolizumab and nivolumab. In certain embodiments, for treatment of cancer and/or inflammatory disease, the subject compounds can be administered in combination with a colony-stimulating factor-1 receptor (CSF1R) inhibitors. CSF1R inhibitors of interest include, but are not limited to, emactuzumab.

Any convenient cancer vaccine therapies and agents can be used in combination with the subject immunomodulatory polypeptide compositions and methods. For treatment of cancer, e.g., ovarian cancer, the subject compounds can be administered in combination with a vaccination therapy, e.g., a dendritic cell (DC) vaccination agent that promotes Th1/Th17 immunity. Th17 cell infiltration correlates with markedly prolonged overall survival among ovarian cancer patients. In some embodiments, the immunomodulatory polypeptide finds use as adjuvant treatment in combination with Th17-inducing vaccination.

Also of interest are agents that are CARP-1/CCARI (Cell division cycle and apoptosis regulator 1) inhibitors, including but not limited to those described by Rishi et al., Journal of Biomedical Nanotechnology, Volume 11, Number 9, September 2015, pp. 1608-1627(20), and CD47 inhibitors, including, but not limited to, anti-CD47 antibody agents such as Hu5F9-G4.

### Pharmaceutical Compositions

Also provided are pharmaceutical compositions that include a subject compound (either alone or in the presence of one or more additional active agents) present in a pharmaceutically acceptable vehicle. The term “pharmaceutically acceptable” means approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a mammal, the compounds and compositions of the invention and pharmaceutically acceptable vehicles, excipients, or diluents may be sterile. In some instances, an aqueous medium is employed as a vehicle when the compound of the invention is administered intravenously, such as water, saline solutions, and aqueous dextrose and glycerol solutions.

Pharmaceutical compositions can take the form of capsules, tablets, pills, pellets, lozenges, powders, granules, syrups, elixirs, solutions, suspensions, emulsions, suppositories, or sustained-release formulations thereof, or any other form suitable for administration to a mammal. In some instances, the pharmaceutical compositions are formulated for administration in accordance with routine procedures as a pharmaceutical composition adapted for oral or intravenous administration to humans. Examples of suitable pharmaceutical vehicles and methods for formulation thereof are described in Remington: The Science and Practice of Pharmacy, Alfonso R. Gennaro ed., Mack Publishing Co. Easton, Pa., 19th ed., 1995, Chapters 86, 87, 88, 91, and 92, incorporated herein by reference.

The choice of excipient will be determined in part by the particular compound, as well as by the particular method used to administer the composition. Accordingly, there is a wide variety of suitable formulations of the pharmaceutical composition of the present invention.

Administration of compounds of the present disclosure may be systemic or local. In certain embodiments administration to a mammal will result in systemic release of a compound of the invention (for example, into the bloodstream). Methods of administration may include enteral routes, such as oral, buccal, sublingual, and rectal; topical administration, such as transdermal and intradermal; and parenteral administration. Suitable parenteral routes include injection via a hypodermic needle or catheter, for example, intravenous, intramuscular, subcutaneous, intradermal, intraperitoneal, intraarterial, intraventricular, intrathecal, and intracameral injection and non-injection routes, such as intravaginal, rectal, or nasal administration. In certain embodiments, the compounds and compositions of the invention are administered orally. In certain embodiments, it may be desirable to administer one or more compounds of the invention locally to the area in need of treatment. This may be achieved, for example, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

The subject compounds can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

In some embodiments, formulations suitable for oral administration can include (a) liquid solutions, such as an effective amount of the compound dissolved in diluents, such as water, or saline; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can include the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are described herein.

The subject formulations can be made into aerosol formulations to be administered via inhalation. These aerosol formulations can be placed into pressurized acceptable propellants, such as dichlorodifluoromethane, propane, nitrogen, and the like. They may also be formulated as pharmaceuticals for non-pressured preparations such as for use in a nebulizer or an atomizer.

In some embodiments, formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Formulations suitable for topical administration may be presented as creams, gels, pastes, or foams, containing, in addition to the active ingredient, such carriers as are appropriate. In some embodiments the topical formulation contains one or more components selected from a structuring agent, a thickener or gelling agent, and an emollient or lubricant. Frequently employed structuring agents include long chain alcohols, such as stearyl alcohol, and glyceryl ethers or esters and oligo(ethylene oxide) ethers or esters thereof. Thickeners and gelling agents include, for example, polymers of acrylic or methacrylic acid and esters thereof, polyacrylamides, and naturally occurring thickeners such as agar, carrageenan, gelatin, and guar gum. Examples of emollients include triglyceride esters, fatty acid esters and amides, waxes such as beeswax, spermaceti, or carnauba wax, phospholipids such as lecithin, and sterols and fatty acid esters thereof. The topical formulations may further include other components, e.g., astringents, fragrances, pigments, skin penetration enhancing agents, sunscreens (e.g., sunblocking agents), etc.

A compound of the present disclosure may also be formulated for oral administration. For an oral pharmaceutical formulation, suitable excipients include pharmaceutical grades of carriers such as mannitol, lactose, glucose, sucrose, starch, cellulose, gelatin, magnesium stearate, sodium saccharine, and/or magnesium carbonate. For use in oral liquid formulations, the composition may be prepared as a solution, suspension, emulsion, or syrup, being supplied either in solid or liquid form suitable for hydration in an aqueous carrier, such as, for example, aqueous saline, aqueous dextrose, glycerol, or ethanol, preferably water or normal saline. If desired, the composition may also contain minor amounts of non-toxic auxiliary substances such as wetting agents, emulsifying agents, or buffers. A compound of the invention may also be incorporated into existing nutraceutical formulations, such as are available conventionally, which may also include an herbal extract.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may include the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Dose levels can vary as a function of the specific compound, the nature of the delivery vehicle, and the like. Desired dosages for a given compound are readily determinable by a variety of means.

The dose administered to an animal, particularly a human, in the context of the present invention should be sufficient to effect a prophylactic or therapeutic response in the animal over a reasonable time frame, e.g., as described in greater detail below. Dosage will depend on a variety of factors including the strength of the particular compound employed, the condition of the animal, and the body weight of the animal, as well as the severity of the illness and the stage of the disease. The size of the dose will also be determined by the existence, nature, and extent of any adverse side-effects that might accompany the administration of a particular compound.

In pharmaceutical dosage forms, the compounds may be administered in the form of a free base, their pharmaceutically acceptable salts, or they may also be used alone or in appropriate association, as well as in combination, with other pharmaceutically active compounds.

In some embodiments, a pharmaceutical composition includes a subject compound that specifically binds with high affinity to a target protein, and a pharmaceutically acceptable vehicle. In certain embodiments, the target protein is a PD-1 protein and the subject compound is a PD-1 antagonist.

### Kits

Also provided are kits that include compounds of the present disclosure. Kits of the present disclosure may include one or more dosages of the compound, and optionally one or more dosages of one or more additional active agents. Conveniently, the formulations may be provided in a unit dosage format. In such kits, in addition to the containers containing the formulation(s), e.g. unit doses, is an informational package insert describing the use of the subject formulations in the methods of the invention, e.g., instructions for using the subject unit doses to treat cellular conditions associated with pathogenic angiogenesis. The term kit refers to a packaged active agent or agents. In some embodiments, the subject system or kit includes a dose of a subject compound (e.g., as described herein) and a dose of a second active agent (e.g., as described herein) in amounts effective to treat a subject for a disease or condition associated with angiogenesis (e.g., as described herein).

In addition to the above-mentioned components, a subject kit may further include instructions for using the components of the kit, e.g., to practice the subject method. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, Hard Disk Drive (HDD), portable flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In some embodiments, a kit includes a first dosage of a subject pharmaceutical composition and a second dosage of a subject pharmaceutical composition. In certain embodiments, the kit further includes a second angiogenesis modulatory agent.

### Utility

The compounds of the invention, e.g., as described above, find use in a variety of applications. Applications of interest include, but are not limited to: therapeutic applications, research applications, and screening applications. Each of these different applications are now reviewed in greater details below.

### Therapeutic Applications

The subject compounds find use in a variety of therapeutic applications. Therapeutic applications of interest include those applications in which the activity of the target is the cause or a compounding factor in disease progression. As such, the subject compounds find use in the treatment of a variety of different conditions in which the modulation of target activity in the host is desired.

The subject compounds are useful for treating a disorder relating to its target, e.g., PD-1. Examples of disease conditions which may be treated with compounds of the disclosure are described herein.

In one embodiment, the present disclosure provides a method of treating a subject for a PD-1-related condition. The method generally involves administering a subject compound to a subject having a PD-1 related disorder in an amount effective to treat at least one symptom of the PD-1 related disorder.

In some embodiments, the subject multimeric compounds are D-peptidic bispecific T cell engagers that find use in any convenient immunotherapeutic applications where antibody based BiTEs find use, including a variety of cancers, such as B cell malignancy, CLL, B-ALL, Leukemia, Lymphoma or solid tumors. Solid tumors of interest include, but are not limited to, solid tumors are selected from breast cancer, prostate cancer, bladder cancer, soft tissue sarcoma, lymphomas, esophageal cancer, uterine cancer, bone cancer, adrenal gland cancer, lung cancer, thyroid cancer, colon cancer, glioma, liver cancer, pancreatic cancer, renal cancer, cervical cancer, testicular cancer, head and neck cancer, ovarian cancer, neuroblastoma and melanoma. In some embodiments, the D-peptidic bispecific T cell engagers include a first monomer that binds to a T cell-specific molecule, usually CD3, and a second monomer that binds to a tumor-associated antigen.

### Research Applications

The subject compounds and methods find use in a variety of research applications. The subject compounds and methods may be used to analyze the roles of target proteins in modulating various biological processes, including but not limited to angiogenesis, inflammation, cellular growth, metabolism, regulation of transcription and regulation of phosphorylation. Other target protein binding molecules such as antibodies have been similarly useful in similar areas of biological research. See e.g., Sidhu and Fellhouse, “Synthetic therapeutic antibodies,” Nature Chemical Biology, 2006, 2(12), 682-688. Such methods can be readily modified for use in a variety of research applications of the subject compounds and methods.

### Diagnostic Applications

The subject compounds and methods find use in a variety of diagnostic applications, including but not limited to, the development of clinical diagnostics, e.g., in vitro diagnostics or in vivo tumor imaging agents. Such applications are useful in diagnosing or confirming diagnosis of a disease condition, or susceptibility thereto. The methods are also useful for monitoring disease progression and/or response to treatment in patients who have been previously diagnosed with the disease.

Diagnostic applications of interest include diagnosis of disease conditions, such as those conditions described above, including but not limited to: cancer, inhibition of angiogenesis and metastasis, osteoarthritis pain, chronic lower back pain, cancer-related pain, age-related macular degeneration (AMD), diabetic macular edema (DME), ideopathic pulmonary fibrosis (IPF) and graft survival of transplanted corneas. In some methods, the same compound can serve as both a treatment and diagnostic reagent.

Other target protein binding molecules, such as aptamers and antibodies, have also found use in the development of clinical diagnostics. Such methods can be readily modified for use in a variety of diagnostics applications of the subject compounds and methods, see for example, Jayasena, “Aptamers: An Emerging Class of Molecules That Rival Antibodies in Diagnostics,” Clinical Chemistry, 1999, 45, 1628-1650.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Definitions

The term “peptidic” refers to a compound, or unit thereof, that is composed primarily of amino acid residues linked together as a polypeptide, or a peptidomimetic compound, or unit thereof, that is capable of mimicking the biological action of a parent polypeptide. A “peptidomimetic” compound is a bioisostere of a parent peptide sequence that contains one or more organic structural elements which mimic at least part of an amino acid residue of the parent peptide and provides a compound having broadly similar biological properties as the parent peptide. Peptidomimetic compounds can have similar target biological activity as compared to a parent peptide compound while providing desirable physical and/or non-target biological properties, such as resistance to proteolytic degradation or increased bioavailability. The terms peptide and polypeptide are used interchangeably herein. The structural elements of a peptidomimetic compound include organic groups designed to mimic a component of a peptide backbone or to mimic an amino acid sidechain. A peptidomimetic generally includes a backbone having a configuration of sidechain groups that mimics those found in a parent polypeptide sequence, and can include sidechain groups not found among the known 20 proteinogenic amino acids, substitutions of the amide bond hydrogen moiety by methyl groups (N-methylation) or other alkyl groups, replacement of a peptide bond with a chemical group or bond that is resistant to chemical or enzymatic treatments, non-peptide-based linkers used to effect cyclization between the ends or internal portions of the molecule, N- and C-terminal modifications, and conjugation with a non-peptidic extension (such as polyethylene glycol, lipids, carbohydrates, nucleosides, nucleotides, nucleoside bases, various small molecules, or phosphate or sulfate groups). A peptidic compound that is composed primarily of amino acid residues can be based on a parent polypeptide sequence having a number of amino acid residues (e.g., 5 or less) replaced with peptidomimetic moiety or peptidomimetic monomer units that mimic amino acid residues. In some embodiments, a peptidic compound that is composed primarily of amino acid residues has 2 residues or less per 10 amino acid residues of a parent polypeptide sequence replaced with a peptidomimetic moiety. Any convenient peptidomimetic groups and chemistries can be utilized in the subject D-peptidic compounds. Any convenient peptidomimetic groups can be utilized in the subject D-peptidic compounds. The term peptidic is meant to include modified peptide compounds where a non-proteinaceous moiety has been covalently linked to the compound (e.g., at a terminal of the compound), compounds that include an N-terminal modification and compounds that include a C-terminal modification.

The term “analog” of an amino acid residue refers to a residue having a sidechain group that is a structural and/or functional analog of the sidechain group of the reference amino acid residue. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as F, Cl or Br, etc.), deletion of a group, substitution of a covalent bond (single bond for double bond, etc.), or combinations thereof. For example, amino acid analogs may include a-hydroxy acids, and a-amino acids, and the like. In some embodiments, an analog of an amino acid residue is a substituted version of the amino acid. The term “substituted version” of an amino acid residue refers to a residue having a sidechain group that includes one or more additional substituents on the sidechain group that are not present in the sidechain of the reference amino acid residue.

The term “avidity” refers to the accumulated strength of multiple affinities of individual non-covalent binding interactions, such as between a protein receptor and its ligand, and is sometimes referred to as functional affinity. Avidity is distinct from affinity, which describes the strength of a single interaction. However, because individual binding events increase the likelihood of other interactions to occur (i.e. increase the local concentration of each binding partner in proximity to the binding site), avidity should not be thought of as the mere sum of its constituent affinities but as the combined effect of all affinities participating in the biomolecular interaction. Avidity can be applied to protein-protein interactions in which multiple target binding sites simultaneously interact with their protein ligands, sometimes in multimerized structures. Individually, each binding interaction may be readily broken; however, when many binding interactions are present at the same time, transient unbinding of a single site does not allow the molecule to diffuse away, and binding of that weak interaction is likely to be restored.

The terms “linker”, “linkage” and “linking group” are used interchangeably and refer to a linking moiety that covalently connects two or more compounds. In some embodiments, the linker is divalent. In certain cases, the linker is a branched or trivalent linking group. In some embodiments, the linker has a linear or branched backbone of 200 atoms or less (such as 100 atoms or less, 80 atoms or less, 60 atoms or less, 50 atoms or less, 40 atoms or less, 30 atoms or less, or even 20 atoms or less) in length. A linking moiety may be a covalent bond that connects two groups or a linear or branched chain of between 1 and 200 atoms in length, for example of about 1, 2, 3, 4, 5, 6, 8, 10, 12, 14, 16, 18, 20, 30, 40, 50, 100, 150 or 200 carbon atoms in length, where the linker may be linear, branched, cyclic or a single atom. In certain cases, one, two, three, four or five or more carbon atoms of a linker backbone may be optionally substituted with a sulfur, nitrogen or oxygen heteroatom. In certain instances, when the linker includes a PEG group, every third atom of that segment of the linker backbone is substituted with an oxygen. The bonds between backbone atoms may be saturated or unsaturated, usually not more than one, two, or three unsaturated bonds will be present in a linker backbone. The linker may include one or more substituent groups, for example an alkyl, aryl or alkenyl group. A linker may include, without limitations, oligo(ethylene glycol), ethers, thioethers, disulfide, amides, carbonates, carbamates, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable. A linker may be peptidic, e.g., a linking sequence of residues.

The terms “polypeptide,” “peptide,” and “protein” are used interchangeably to refer to a polymeric form of amino acids of any length. Unless specifically indicated otherwise, “polypeptide,” “peptide,” and “protein” can include naturally occurring amino acids in L-form, or a D-enantiomer thereof, chemically or biochemically modified or derivatized amino acids. A polypeptide may be of any convenient length, e.g., 2 or more amino acids, 4 or more amino acids, 10 or more amino acids, 20 or more amino acids, 30 or more amino acids, 40 or more amino acids, 50 or more amino acids, 60 or more amino acids, 100 or more amino acids, 300 or more amino acids, 500 or more or 1000 or more amino acids. In some embodiments, the term “peptide” can be used to refer to a smaller polypeptide, e.g., 20 or less amino acids, such as 10 or less amino acids, and the term “protein” can be used to refer to a larger polypeptide, e.g., 30 or more amino acids, such as 40 or more amino acids, that is capable of folding to produce a three dimensional structure.

For the polypeptide sequences and motifs depicted herein, unless noted otherwise, capital letter codes refer to L-amino acid residues and small letter codes refer to D-amino acid residues. The amino acid residue glycine is represented as G or Gly. “a” is alanine. “c” is cysteine. “d” is aspartic acid. “e” is glutamic acid. “f” is phenylalanine. “h” is histidine. “i” is isoleucine. “k” is lysine. “1” is leucine. “m” is methionine. “n” is asparagine. “o” is ornithine. “p” is proline. “q” is glutamine. “r” is arginine. “s” is serine. “t” is threonine. “v” is valine. “w” is tryptophan. “y” is tyrosine. It is understood that for any of the sequences and motifs described herein, e.g., sequences defining a D-peptidic compound that specifically binds PD-1, a mirror image compound is also encompassed which specifically binds to the mirror image of PD-1. The present disclosure is meant to encompass both versions of the subject compounds, e.g., L-peptidic compounds that specifically bind D-PD-1 and D-peptidic compounds that specifically bind L-PD-1. It is understood that D-PD-1 protein may be targeted primarily in a variety of in vitro applications, while L-PD-1 protein may be targeted for a variety of in vitro and/or in vivo applications.

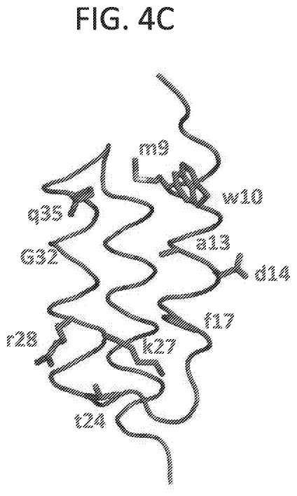

The terms “scaffold” and “scaffold domain” are used interchangeably and refer to a reference D-peptidic framework motif from which a subject D-peptidic compound arose, or against which the subject D-peptidic compound is able to be compared, e.g., via a sequence or structural alignment method. The structural motif of a scaffold domain can be based on a naturally occurring protein domain structure. For a particular protein domain structural motif, several related underlying sequences may be available, any one of which can provide for the particular three-dimensional structure of the scaffold domain. A scaffold domain can be defined in terms of a characteristic consensus sequence motif. FIG. 6 shows one possible consensus sequence for a GA scaffold domain based on an alignment and comparison of 16 related naturally occurring protein domain sequences which provide for the three-helix bundle structural motif of a GA scaffold domain.

A compound that “specifically binds” to an epitope or binding site of a target protein is a term well understood in the art, and methods to determine such specific or preferential binding are also well known in the art. A compound exhibits “specific binding” if it associates more frequently, more rapidly, with greater duration and/or with greater affinity with a particular cell or substance (target protein) than it does with alternative cells or substances. A D-peptidic compound “specifically binds” to a target if it binds with greater affinity, avidity, more readily, and/or with greater duration than it binds to other substances. For example, a compound that specifically or preferentially binds to a PD-1 epitope or site is an antibody that binds this epitope or site with greater affinity, avidity, more readily, and/or with greater duration than it binds to other PD-1 epitopes or non-PD-1 epitopes. It is also understood by reading this definition that, for example, a compound that specifically or preferentially binds to a first target may or may not specifically or preferentially bind to a second target. As such, “specific binding” does not necessarily require (although it can include) exclusive binding. Generally, but not necessarily, reference to binding means specific binding.

A “specificity determining motif” refers to an arrangement of variant amino acids incorporated at particular locations of a variant scaffold domain that provides for specific binding of the variant domain to a target protein. The motif can encompass continuous and/or a discontinuous sequences of residues. The motif can encompass variant amino acids located at one face of the compound structure and which are capable of contacting the target protein, or can encompass variant residues which do not provide contacts with the target but rather provide for a modification to the natural domain structure that enhances binding to the target. The motif may be considered to be incorporated into, or integrated with, an underlying scaffold domain structure or sequence, e.g., a three helix bundle of a naturally occurring GA or Z domain.

As used herein, the terms “variant amino acid” and “variant residue” are used interchangeably to refer to the particular residues of a subject compound which are modified or mutated by comparison to an underlying scaffold domain. The variant residues encompass those residues that were selected (e.g., via mirror image screening, affinity maturation and/or point mutation(s)) to provide for a desirable domain motif structure that specific binds to the target. When a compound includes amino acid mutations or modifications at particular positions by comparison to a scaffold domain, the amino acid residues of the D-peptidic compound located at those particular positions are referred to as “variant amino acids.” Such variant amino acids may confer on the resulting D-peptidic compounds different functions, such as specific binding to a target protein, increased water solubility, ease of chemical synthesis, metabolic stability, etc. Aspects of the present disclosure include D-peptidic compounds that were selected from a phage display library based on a GA scaffold domain and further developed (e.g., via additional affinity maturation and/or point mutations), and as such include several variant amino acids integrated with a GA scaffold domain.

The term “helix-terminating residue” refers to an amino acid residue that has a high free energy penalty for forming a helix structure relative to an analogous alanine residue. In some embodiments, a high free energy helix penalty is referred to as a helix propensity value and is 0.5 kcal/mol or greater as defined by the method of Pace and Scholtz where higher values indicate increased penalty (“A Helix Propensity Scale Based on Experimental Studies of Peptides and Proteins”, Biophysical Journal Volume 75 July 1998 422-427). In some embodiments, a helix-terminating residue is a naturally occurring residue that has a helix propensity value of 0.5 or more (kcal/mol), such as 0.55 or more, 0.60 or more, 0.65 or more or 0.70 or more. For example, proline has a helix propensity value of 3.16 kcal/mol and glycine has a helix propensity value of 1.00 kcal/mol, as shown in Table 1. The helix propensity values of non-naturally occurring helix-terminating residues may be estimated by using the value of the closest naturally occurring residue having a sidechain group that is a structural analog.

TABLE 4
Naturally occurring amino
acid alpha-helical propensities

| | | Helix |
|---|---|---|
| | | propensity |
| | | value |
| 3-Letter | 1-Letter | (kcal/mol)* |
| Ala | A | 0 |
| Arg | R | 0.21 |
| Asn | N | 0.65 |
| Asp | D | 0.69 |
| Cys | C | 0.68 |
| Glu | E | 0.40 |
| Gln | Q | 0.39 |
| Gly | G | 1.00 |
| His | H | 0.61 |
| Ile | I | 0.41 |
| Leu | L | 0.21 |
| Lys | K | 0.26 |
| Met | M | 0.24 |
| Phe | F | 0.54 |
| Pro | P | 3.16 |
| Ser | S | 0.50 |
| Thr | T | 0.66 |
| Trp | W | 0.49 |
| Tyr | Y | 0.53 |
| Val | V | 0.61 |

*Estimated differences in free energy, estimated in kcal/mol per residue in an alpha-helical configuration, relative to Alanine arbitrarily set as zero. Higher numbers (more positive free energies) are less favored. In some embodiments, deviations from these average numbers are possible, depending on the identities of the neighboring residues.

As used herein, “similar,” “conservative,” and “highly conservative” amino acid substitutions are defined as shown in Table 5, below. The determination of whether an amino acid residue substitution is similar, conservative, or highly conservative can be based on the side chain of the amino acid residue and not the polypeptide backbone.

TABLE 5
Classification of Amino Acid Substitutions

| | | | Highly |
|---|---|---|---|
| Amino Acid | Similar | Conservative | Conservative |
| in Subject | Amino Acid | Amino Acid | Amino Acid |
| Polypeptide | Substitutions | Substitutions | Substitutions |
| Glycine (G) | A, S, N | A | n/a |
| Alanine (A) | S, G, T, V, C, P, Q | S, G, T | S |
| Serine (S) | T, A, N, G, Q | T, A, N | T, A |
| Threonine (T) | S, A, V, N, M | S, A, V, N | S |
| Cysteine (C) | A, S, T, V, I | A | n/a |
| Proline (P) | A, S, T, K | A | n/a |
| Methionine (M) | L, I, V, F | L, I, V | L, I |
| Valine (V) | I, L, M, T, A | I, L, M | I |
| Leucine (L) | M, I, V, F, T, A | M, I, V, F | M, I |
| Isoleucine (I) | V, L, M, F, T, C | V, L, M, F | V, L, M |
| Phenylalanine (F) | W, Y, L, M, I, V | W, L | n/a |
| Tyrosine (Y) | F, W, H, L, I | F, W | F |
| Tryptophan (W) | F, L, V | F | n/a |
| Asparagine (N) | Q | Q | Q |
| Glutamine (Q) | N | N | N |
| Aspartic Acid (D) | E | E | E |
| Glutamic Acid (E) | D | D | D |
| Histidine (H) | R, K | R, K | R, K |
| Lysine (K) | R, H, O | R, H, O | R, O |
| Arginine (R) | K, H, O | K, H, O | K, O |
| Ornithine (O) | R, H, K | R, H, K | K, R |

The term “stable” refers to a compound that is able to maintain a folded state under physiological conditions at a certain temperature, such that it retains at least one of its normal functional activities, for example binding to a target protein. The stability of the compound can be determined using standard methods. For example, the “thermostability” of a compound can be determined by measuring the thermal melt (“Tm”) temperature. The Tm is the temperature in degrees Celsius at which half of the compound becomes unfolded. In some instances, the higher the Tm, the more stable the compound.

The term “a target protein” refers to all members of the target family, and fragments and enantiomers thereof, and protein mimics thereof. The target proteins of interest that are described herein are intended to include all members of the target family, and fragments and enantiomers thereof, and protein mimics thereof, unless explicitly described otherwise. The target protein may be any protein of interest, such as a therapeutic or diagnostic target. The term “target protein” is intended to include recombinant and synthetic molecules, which can be prepared using any convenient recombinant expression methods or using any convenient synthetic methods, or purchased commercially, as well as fusion proteins containing a target molecule, as well as synthetic L- or D-proteins.

The term “VEGF” or its non-abbreviated form “vascular endothelial growth factor”, as used herein, refers to the protein products encoded by the VEGF gene. The term VEGF includes all members of the VEGF family, such as, VEGF-A, VEGF-B, VEGF-C, VEGF-D, VEGF-E, and fragments and enantiomers thereof. The term VEGF is intended to include recombinant and synthetic VEGF molecules, which can be prepared using any convenient recombinant expression methods or using any convenient synthetic methods, or purchased commercially (e.g. R & D Systems, Catalog No. 210-TA, Minneapolis, Minn.), as well as fusion proteins containing a VEGF molecule, as well as synthetic L- or D-proteins. VEGF is involved in both vasculogenesis (the de novo formation of the embryonic circulatory system) and angiogenesis (the growth of blood vessels from pre-existing vasculature) and can also be involved in the growth of lymphatic vessels in a process known as lymphangiogenesis. Members of the VEGF family stimulate cellular responses by binding to tyrosine kinase receptors (the VEGFRs) on the cell surface, causing them to dimerize and become activated through transphosphorylation. The VEGF receptors have an extracellular portion containing 7 immunoglobulin-like domains, a single transmembrane spanning region and an intracellular portion containing a split tyrosine-kinase domain. VEGF-A binds to VEGFR-1 (Flt-1) and VEGFR-2 (KDR/Flk-1). VEGFR-2 appears to mediate several of the cellular responses to VEGF. VEGF, its biological activities, and its receptors are well studied and are described in Matsumoto et al. (VEGF receptor signal transduction Sci STKE. 2001:RE21 and Marti et al (Angiogenesis in ischemic disease. Thromb Haemost. 1999 Suppl 1:44-52). Amino acid sequences of exemplary VEGFs are found in the NCBI's Genbank database and a full description of VEGF proteins and their roles in various diseases and conditions is found in NCBI's Online Mendelian Inheritance in Man database.

### Exemplary Embodiments

Aspects of the present disclosure are embodied in the clauses and exemplary embodiments set forth below.

Clause 1. A multivalent D-peptidic compound, comprising:
(a) a first D-peptidic domain that specifically binds a target protein; and
(b) a second D-peptidic domain that specifically binds the target protein at a distinct binding site on the target protein that is non-overlapping with the binding site bound by the first D-peptidic domain; and
(c) a linking component that covalently links the first and second D-peptidic domains such that the first and second D-peptidic domains are capable of simultaneously binding the target protein.
   Clause 2. The D-peptidic compound of clause 1, wherein:

the first D-peptidic domain is a first three-helix bundle domain capable of specifically binding a first binding site of the target protein; and

the second D-peptidic domain is a second three-helix bundle domain capable of specifically binding a second binding site of the target protein.

Clause 3. The D-peptidic compound of clause 1, wherein the first and second D-peptidic domains are selected from D-peptidic GA domain and D-peptidic Z domain.
Clause 4. The D-peptidic compound of any one of clauses 1-3, wherein:

the first D-peptidic domain is a D-peptidic GA domain; and the second D-peptidic domain is a D-peptidic Z domain.

Clause 5. The D-peptidic compound of any one of clauses 1-4, wherein the compound is bivalent.
Clause 6. The D-peptidic compound of any one of clauses 1-4, wherein the compound further comprises a third D-peptidic domain that specifically binds a target protein (e.g., trivalent, tetravalent, etc.).
Clause 7. The D-peptidic compound of any one of clauses 1-6, that specifically binds the target protein with a binding affinity (K_{D}) 10-fold or more (e.g., 30-fold or more, 100-fold or more, 300-fold or more or 1000-fold or more, as measured by SPR) stronger than each of the binding affinities of the first and second D-peptidic domains alone for the target protein.
Clause 8. The D-peptidic compound of clause 7, wherein:

the compound has a binding affinity (K_{D}) for the target protein of 3 nM or less (e.g., 1 nM or less, 300 μM or less, 100 μM or less); and

the binding affinities of the first and second D-peptidic domains alone for the target protein are each independently 100 nM or more (e.g., 300 nM or more, 1 uM or more).

Clause 9. The D-peptidic compound of clause 7 or 8, having in vitro antagonist activity (IC₅₀) against the target protein that is at least 10-fold more potent (e.g., at least 30-fold, at least 100-fold, at least 300-fold, etc. as measured by ELISA assay as described herein) than each of the first and second D-peptidic domains alone.
Clause 10. The D-peptidic compound of any one of clauses 1-9, wherein the first D-peptidic domain consists essentially of a single chain polypeptide sequence of 30 to 80 residues (e.g., 40 to 70, 45 to 60 residues, 50 to 60 residues, or 52 to 58 residues), and has a MW of 1 to 10 kDa (e.g., 2 to 8 kDa, 3 to 8 kDa or 4 to 6 kDa).
Clause 11. The D-peptidic compound of any one of clauses 1-10, wherein the second D-peptidic domain consists essentially of a single chain polypeptide sequence of 30 to 80 residues (e.g., 40 to 70, 45 to 60 residues, 50 to 60 residues, or 52 to 58 residues), and has a MW of 1 to 10 kDa (e.g., 2 to 8 kDa, 3 to 8 kDa or 4 to 6 kDa).
Clause 12. The D-peptidic compound of any one of clauses 1-11, wherein the linking component is a linker connecting a terminal amino acid residue of the first D-peptidic domain to a terminal amino acid residue of the second D-peptidic domain (e.g., N-terminal to N-terminal linker or C-terminal to C-terminal linker).
Clause 13. The D-peptidic compound of clause 12, wherein the linking component is a linker connecting an amino acid sidechain of the first D-peptidic domain to a terminal amino acid residue of the second D-peptidic domain that are in proximity to each other when the first and second D-peptidic domains are simultaneously bound to the target protein.
Clause 14. The D-peptidic compound of clause 13, wherein the linking component is a linker connecting an amino acid sidechain of the first D-peptidic domain to a proximal amino acid sidechain of the second D-peptidic domain when the first and second D-peptidic domains are simultaneously bound to the target protein.
Clause 15. The D-peptidic compound of any one of clauses 1-14, wherein the linking component comprises one or more groups selected from amino acid residue, polypeptide, (PEG)˜linker (e.g., n is 2-50, 3-50, 4-50, 6-50 or 6-20), modified PEG moiety, C₍₁₋₆₎alkyl linker, substituted C₍₁₋₆₎alkyl linker, —CO(CH₂)ₘCO—, —NR(CH₂)ₚNR—, —CO(CH₂)ₘNR—, —CO(CH₂)ₘO—, —CO(CH₂)ₘS—, and linked chemoselective functional groups (e.g., —CONH—, —OCONH—, click chemistry conjugate such as 1,2,3-triazole, maleimide-thiol conjugate thiosuccinimide, haloacetyl-thiol conjugate thioether, etc.), wherein m is 1 to 6, p is 2-6 and each R is independently H, C₍₁₋₆₎alkyl or substituted C₍₁₋₆₎alkyl.
Clause 16. The D-peptidic compound of any one of clauses 1-15, wherein the target protein is monomeric.
Clause 17. The D-peptidic compound of any one of clauses 1-16, wherein the target protein is dimeric.
Clause 18. The D-peptidic compound of clause 16 or 17, wherein the compound further comprises a third D-peptidic domain that is homologous to the first D-peptidic domain.
Clause 19. The D-peptidic compound of clause 18, wherein the compound further comprises a fourth D-peptidic domain that is homologous to the second D-peptidic domain.
Clause 20. The D-peptidic compound of clause 19, wherein the D-peptidic domains are configured as a dimer of a bivalent moiety comprising first and second D-peptidic domains.
Clause 21. The D-peptidic compound of any one of clauses 1-20, wherein the target protein is PD1.
Clause 22. The D-peptidic compound of clause 2, wherein:

the target protein is PD1;

the first binding site is non-overlapping with the PD-L1 binding site on PD-1; and

the second binding site overlaps at least partially with the PD-L1 binding site on PD-1.

Clause 23. The D-peptidic compound of clause 22, wherein the first binding site comprises the amino acid sidechains S38, P39, A40, T53, S55, L100, P101, N102, R104, D105 and H107 of PD-1.
Clause 24. The D-peptidic compound of clause 22 or 23, wherein the second binding site comprises the amino acid sidechains V64, N66, Y68, M70, T76, K78, 1126, L128, A132, Q133, 1134 and E136 of PD-1.
Clause 25. The D-peptidic compound of any one of clauses 21-24, wherein the first D-peptidic domain is linked to the second D-peptidic domain via a N-terminal to N-terminal linker.
Clause 26. The D-peptidic compound of clause 25, wherein the N-terminal to N-terminal linker is a (PEG)ₙ bifunctional linker, wherein n is 2-20 (e.g., n is 3-12 or 6-8, such as 3, 4, 5, 6, 7, 8, 9 or 10).
Clause 27. The D-peptidic compound of any one of clauses 1-26, wherein the first D-peptidic domain is a D-peptidic GA domain polypeptide having a specificity-determining motif (SDM) comprising 5 or more (e.g., 5, 6, 7, 8, 9, 10, 11, 12, 13, 14, 15 or 16) variant amino acid residues at positions selected from 25, 27, 30, 31, 34, 36, 37, 39, 40 and 42-48.
Clause 28. The D-peptidic compound of any one of clauses 1-27, wherein the second D-peptidic domain is a D-peptidic Z domain having a specificity-determining motif (SDM) comprising 5 or more variant amino acid residues (e.g., 6 or more, such as 6, 7, 8, 9 or 10) at positions selected from 9, 10, 13, 14, 17, 24, 27, 28, 32 and 35.
Clause 29. The multivalent D-peptidic compound of clause 21 that specifically binds PD-1, comprising:

(a) a D-peptidic GA domain capable of specifically binding a first binding site of PD-1; and

(b) a D-peptidic Z domain capable of specifically binding a second binding site of PD-1.

Clause 30. The D-peptidic compound of clause 29, wherein the linking component covalently links the D-peptidic GA and Z domains.
Clause 31. The D-peptidic compound of clause 30, wherein the linking component is configured to link the D-peptidic GA and Z domains whereby the domains are capable of simultaneously binding to PD1.
Clause 32. The D-peptidic compound of clause 31, wherein the linking component is configured to connect the D-peptidic GA and Z domains via sidechain and/or terminal groups that are proximal to each other when the D-peptidic GA and Z domains are simultaneously bound to PD1.
Clause 33. The D-peptidic compound of any one of clauses 29-32, wherein the linking component comprises a linker connecting a terminal of the D-peptidic GA domain to a terminal of the D-peptidic Z domain.
Clause 34. The D-peptidic compound of clause 29, wherein the linker connects the N-terminal residue of the D-peptidic GA domain polypeptide to the N-terminal residue of the D-peptidic Z domain polypeptide.
Clause 35. The D-peptidic compound of any one of clauses 30-34, wherein the linking component connects a first amino acid sidechain of a residue of the D-peptidic GA domain and a second amino acid sidechain of a residue of the D-peptidic Z domain.
Clause 36. The D-peptidic compound of any one of clauses 30-35, wherein the linking component comprises one or more groups selected from amino acid residue, polypeptide, (PEG)˜linker (e.g., n is 2-50, 3-50, 4-50, 6-50 or 6-20), modified PEG moiety, C₍₁₋₆₎alkyl linker, substituted C₍₁₋₆₎alkyl linker, —CO(CH₂)ₘCO—, —NR(CH₂)ₚNR—, —CO(CH₂)ₘNR—, —CO(CH₂)ₘO—, —CO(CH₂)ₘS—, and linked chemoselective functional groups (e.g., —CONH—, —OCONH—, click chemistry conjugate such as 1,2,3-triazole, maleimide-thiol conjugate thiosuccinimide, haloacetyl-thiol conjugate thioether, etc.), wherein m is 1 to 6, p is 2-6 and each R is independently H, C₍₁₋₆₎alkyl or substituted C₍₁₋₆₎alkyl.
Clause 37. The D-peptidic compound of any one of clauses 30-36, wherein the D-peptidic GA domain and the D-peptidic Z domain are conjugated to each other via N-terminal cysteine residues with a bis-maleimide linker or bis-haloacetyl linker, optionally comprising a (PEG)ₙ moiety (e.g., n is 2-12, such as 3-8, e.g., a PEG3, PEG6, or PEG8 containing linker).
Clause 38. The D-peptidic compound of clause 37, wherein the linking component connecting the D-peptidic GA and Z domains is selected from:

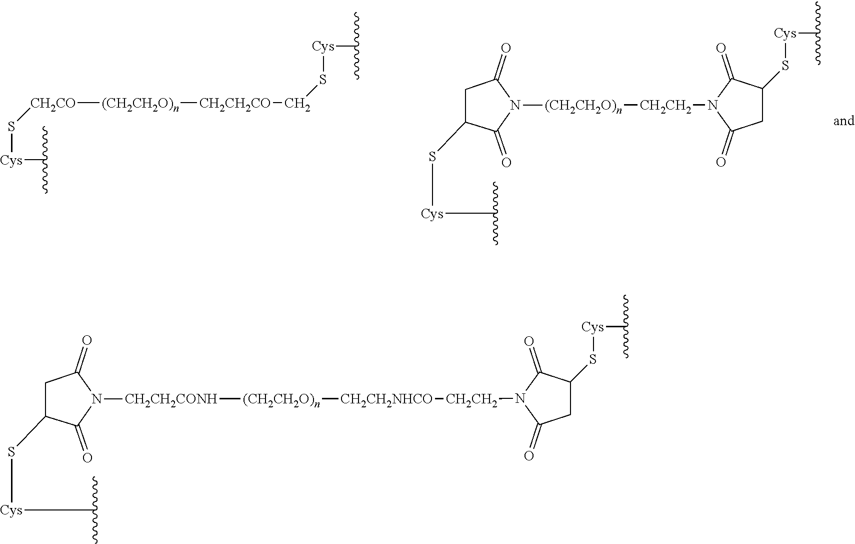

wherein n is 1-20 (e.g., 2 to 12, 2 to 8, or 3 to 6).
Clause 39. The D-peptidic compound of any one of clauses 30-38, wherein the D-peptidic GA domain is according to any one of clauses 48-56.
Clause 40. The D-peptidic compound of clause 39, wherein the D-peptidic GA domain comprises a polypeptide of the sequence:

tidgwllknakedaiaelkkaGitsdlyfnwinvaGsvssvnfhknyilkaha (SEQ ID NO: 32).

Clause 41. The D-peptidic compound of any one of clauses 30-40, wherein the D-peptidic Z domain is according to any one of clauses 57-69.
Clause 42. The D-peptidic compound of clause 41, wherein the D-peptidic Z domain comprises a polypeptide of the sequence:

vdnkfnkemwnaadeifhlpnlnteqkrafiGslqddpsgsanllaeakklndaqapk (SEQ ID NO: 40).

Clause 43. The D-peptidic compound of clause 42, comprising the following polypeptides:

tidgwllknakedaiaelkkaGitsdlyfnwinvaGsvssvnfhknyilkaha (SEQ ID NO: 65); and

vdnkfnkemwnaadeifhlpnlnteqkrafiGslqddpsgsanllaeakklndaqapk (SEQ ID NO: 66);

wherein the polypeptides are linked via the N-terminal cysteine residues with a bis-maleimide bifunctional linking moiety comprising PEG3, PEG6 or PEG8.
Clause 44. The D-peptidic compound of any one of clauses 30-43, wherein the compound further comprises a second GA domain that is homologous to the first GA domain.
Clause 45. The D-peptidic compound of any one of clauses 30-44, wherein the compound further comprises a second Z domain that is homologous to the first Z domain.
Clause 46. A D-peptidic compound that specifically binds PD-1, comprising:

a D-peptidic GA domain comprising:

a) a PD-1 specificity-determining motif (SDM) defined by the following amino acid residues:

| (SEQ ID NO: 67) | |
|---|---|
| | s²⁵-l²⁷---w³¹--x³⁴-x³⁶s³⁷-s³⁹s⁴⁰--x⁴³h⁴⁴--x⁴⁷ |

wherein:
x³⁴ is selected from v and d;
x³⁶ is selected from G and s;
x⁴³ is selected from f and y; and
x⁴⁷ is selected from f and y; or

b) a PD-1 SDM having 80% or more (e.g., 90% or more) identity with the SDM residues defined in (a); or

c) a PD-1 SDM having 1 to 3 amino acid residue substitutions relative to the SDM residues defined in (a), wherein the 1 to 3 amino acid residue substitutions are selected from:
i) a similar amino acid residue substitution according to Table 1;
ii) a conservative amino acid residue substitution according to Table 1;
iii) a highly conserved amino acid residue substitution according to Table 1; and
iv) an amino acid residue substitution according to the motif defined in FIG. 3A or FIG. 50A.
   Clause 47. The D-peptidic compound of clause 46, wherein the SDM residues defined in (a) are:

| (SEQ ID NO: 68) | |
|---|---|
| | s²⁵-l²⁷---w³¹--v³⁴-G³⁶s³⁷-s³⁹s⁴⁰--x⁴³h⁴⁴--y⁴⁷ |

wherein x⁴³ is selected from f and y.

Clause 48. The D-peptidic compound of clause 47, wherein the PD-1 SDM is defined by the following residues:

| (SEQ ID NO: 69) | |
|---|---|
| | s²⁵-l²⁷---w³¹--v³⁴-G³⁶s³⁷-s³⁹s⁴⁰--f⁴³h⁴⁴--y⁴⁷ |
| | or |
| (SEQ ID NO: 70) | |
| | s²⁵-l²⁷---w³¹--v³⁴-G³⁶s³⁷-s³⁹s⁴⁰--y⁴³h⁴⁴--y⁴⁷. |

Clause 49. The D-peptidic compound of any one of clauses 46-48, wherein the SDM residues are comprised in a polypeptide comprising:

a) peptidic framework residues defined by the following amino acid residues:

| (SEQ ID NO: 71) | |
|---|---|
| | -d²⁶-y²⁸fn-i³²n-a³⁵--v³⁸--v⁴¹n-k⁴⁵n-; |

b) peptidic framework residues having 80% or more (e.g., 90% or more) identity with the residues defined in (a); or

c) peptidic framework residues having 1 to 3 amino acid residue substitutions relative to the residues defined in (a), wherein the 1 to 3 amino acid residue substitutions are selected from:
i) a similar amino acid residue substitution according to Table 1;
ii) a conservative amino acid residue substitution according to Table 1; and
iii) a highly conserved amino acid residue substitution according to Table 1.
   Clause 50. The D-peptidic compound of any one of clauses 46-49, comprising a SDM-containing sequence having 80% or more (e.g., 85% or more, 90% or more, or 95% or more) identity to the amino acid sequence:

(SEQ ID NO: 52)
s²⁵dlyfnwinx³⁴ax³⁶svssvnx⁴³hknx⁴⁷;

wherein:

x³⁴ is selected from v and d;

x³⁶ is selected from G and s;

x⁴³ is selected from f and y; and

x⁴⁷ is selected from f and y.

Clause 51. The D-peptidic compound of any one of clauses 46-50, wherein the D-peptidic GA domain comprises a three-helix bundle of the structural formula:

[Helix 1^{(#6-21)}]-[Linker 1^{(#22-26)}]-[Helix 2^{(#27-35)}]-[Linker 2^{(#36-37)}]-[Helix 3^{(#38-51)}]

wherein:

# denotes reference positions of amino acid residues comprised in the D-peptidic GA domain; and

Helix 1^{(#6-21)} comprises a peptidic framework sequence selected from: a)

| (SEQ ID NO: 53) | |
|---|---|
| | l⁶lknakedaiaelkka²¹; |

b) a sequence having 70% or more (e.g., 75% or more, 80% or more, 85% or more, or 90% or more) identity to the amino acid sequence set forth in (a); and

c) a sequence having 1 to 5 amino acid residue substitutions relative to the sequence defined in (a), wherein the 1 to 5 amino acid residue substitutions are selected from:
i) a similar amino acid residue substitution according to Table 1;
ii) a conservative amino acid residue substitution according to Table 1; and
iii) a highly conserved amino acid residue substitution according to Table 1.
   Clause 52. The D-peptidic compound of clause 51, wherein the D-peptidic GA domain further comprises one or more segments of a peptidic framework sequence selected from: a)

| | N-terminal segment: |
|---|---|
| (SEQ ID NO: 54) | |
| | t¹idqw⁵; |
| | Loop 1 segment: |
| (SEQ ID NO: 55) | |
| | G²²it²⁴; |
| | and |
| | C-terminal segment: |
| (SEQ ID NO: 56) | |
| | i⁴⁸lkaha⁵³; |
| | or |

b) one or more segments having 60% or more sequence identity relative to the one or more segments defined in (a); or

c) one or more segments each independently having 0 to 3 amino acid substitutions relative to the segments defined in (a), wherein the 0 to 3 amino acid substitutions are selected from:
i) a similar amino acid residue substitution according to Table 1;
ii) a conservative amino acid residue substitution according to Table 1; and
iii) a highly conserved amino acid residue substitution according to Table 1.
   Clause 53. The D-peptidic compound of any one of clauses 46-52, wherein the D-peptidic GA domain comprises:

(a) a sequence selected from one of compounds 977296 to 977299 (SEQ ID NOs: 32-35);

(b) a sequence having 80% or more identity with the sequence defined in (a); or

(c) a sequence having 1 to 10 (e.g., 1 to 6, 1 to 5, 1 to 4, 1 to 3, 1 to 2, 2 or 1) amino acid residue substitution(s) relative to the sequence defined in (a), wherein the 1 to 10 amino acid substitutions are:
i) a similar amino acid residue substitution according to Table 1;
ii) a conservative amino acid residue substitution according to Table 1; or
iii) a highly conserved amino acid residue substitution according to Table 1.
   Clause 54. The D-peptidic compound of clause 53, wherein the D-peptidic GA domain comprises a polypeptide of one of compounds 977296 to 977299 (SEQ ID NOs: 32-35).
   Clause 55. The D-peptidic compound of any one of clauses 46-54, wherein the compound is dimeric.
   Clause 56. The D-peptidic compound of any one of clauses 46-54, further comprising a second D-peptidic GA domain that is homologous to the first D-peptidic GA domain.
   Clause 57. A D-peptidic compound that specifically binds PD-1, comprising:

a D-peptidic Z domain comprising:

a) a PD-1 specificity-determining motif (SDM) defined by the following amino acid residues:

| (SEQ ID NO: 72) | |
|---|---|
| | x⁹w¹⁰--x¹³d¹⁴--x¹⁷------x²⁴--x²⁷x²⁸---x³²--x³⁵ |

wherein:
x⁹ is selected from k, l and m;
x¹³ is selected from a and G;
x¹⁷ is selected from f and v;
x²⁴ is selected from k, l, m, r, t and v;
x²⁷ is selected from k and r;
x²⁸ is selected from a, G, q, r and s;
x³² is selected from a, G and s; and
x³¹ is selected from d, e, q and t;

b) a PD-1 SDM having 80% or more, or 90% or more identity with the SDM residues defined in (a); or

c) a PD-1 SDM having 1 to 3 amino acid residue substitutions relative to the SDM residues defined in (a), wherein the 1 to 3 amino acid residue substitutions are selected from:
i) a similar amino acid residue substitution according to Table 1;
ii) a conservative amino acid residue substitution according to Table 1;
iii) a highly conserved amino acid residue substitution according to Table 1; and
iv) an amino acid residue substitution according to the SDM defined in FIG. 4A or FIG. 51.
   Clause 58. The D-peptidic compound of clause 57, wherein the SDM residues defined in (a) are:

| (SEQ ID NO: 73) | |
|---|---|
| | m⁹w¹⁰--x¹³d¹⁴--f¹⁷------x²⁴--k²⁷x²⁸---x³²--x³⁵ |
| | or |
| (SEQ ID NO: 74) | |
| | m⁹w¹⁰--a¹³d¹⁴--f¹⁷------x²⁴--k²⁷x²⁸---x³²--x³⁵ |
| | or |
| (SEQ ID NO: 75) | |
| | x⁹w¹⁰--x¹³d¹⁴--x¹⁷------t²⁴--x²⁷r²⁸---G³²--q³⁵ |

wherein:
x⁹ is selected from k, l and m;
x¹³ is selected from a and G;
x¹⁷ is selected from f and v;
x²⁴ is selected from k, r and t;
x²⁷ is selected from k and r;
x²⁸ is selected from r and s;
x³² is selected from a and G; and
x³⁵ is selected from d and q.
   Clause 59. The D-peptidic compound of clause 57 or 58, wherein the SDM residues defined in (a) are:

| (SEQ ID NO: 76) | |
|---|---|
| | m⁹w¹⁰--a¹³d¹⁴--f¹⁷------t²⁴--k²⁷r²⁸---G³²--q³⁵ |
| | or |
| (SEQ ID NO: 77) | |
| | m⁹w¹⁰--G¹³d¹⁴--f¹⁷------r²⁴--k²⁷s²⁸---a³²--d³⁵ |
| | or |
| (SEQ ID NO: 78) | |
| | m⁹w¹⁰--G¹³d¹⁴--f¹⁷------t²⁴--k²⁷r²⁸---G³²--q³⁵ |
| | or |
| (SEQ ID NO: 79) | |
| | m⁹w¹⁰--G¹³d¹⁴--f¹⁷------k²⁴--k²⁷r²⁸---a³²--q³⁵. |

Clause 60. The D-peptidic compound of clause 59, wherein the PD-1 SDM is defined by the following residues:

| (SEQ ID NO: 80) | |
|---|---|
| | m⁹w¹⁰--a¹³d¹⁴--f¹⁷------t²⁴--k²⁷r²⁸---G³²--q³⁵ |

Clause 61. The D-peptidic compound of clause 59, wherein the PD-1 SDM is defined by the following residues:

| (SEQ ID NO: 81) | |
|---|---|
| | m⁹w¹⁰--G¹³d¹⁴--f¹⁷------r²⁴--k²⁷s²⁸---a³²--d³⁵ |
| | or |
| (SEQ ID NO: 82) | |
| | m⁹w¹⁰--G¹³d¹⁴--f¹⁷------t²⁴--k²⁷r²⁸---G³²--q³⁵ |
| | or |
| (SEQ ID NO: 83) | |
| | m⁹w¹⁰--G¹³d¹⁴--f¹⁷------k²⁴--k²⁷r²⁸---a³²--q³⁵. |

Clause 62. The D-peptidic compound of any one of clauses 57-61, wherein the SDM residues are comprised in a polypeptide comprising:

a) peptidic framework residues defined by the following amino acid residues:

| (SEQ ID NO: 84) | |
|---|---|
| | --n¹¹a--e¹⁵i-h¹⁸lpnln-e²5q--a²⁹fi-s³³l-; |

b) peptidic framework residues having 80% or more (e.g., 90% or more) identity with the residues defined in (a); or

c) peptidic framework residues having 1 to 3 amino acid residue substitutions relative to the residues defined in (a), wherein the 1 to 3 amino acid residue substitutions are selected from:
i) a similar amino acid residue substitution according to Table 1;
ii) a conservative amino acid residue substitution according to Table 1; and
iii) a highly conserved amino acid residue substitution according to Table 1.
   Clause 63. The D-peptidic compound of any one of clauses 57-62, comprising a SDM-containing sequence having 80% or more (e.g., 85% or more, 90% or more, or 95% or more) identity to the amino acid sequence:

(SEQ ID NO: 57)
x⁹wnax¹³deix¹⁷hlpnlnx²⁴eqx²⁷x²⁸afix³²slx³⁵.

wherein:

x⁹ is selected from k, l and m;

x¹³ is selected from a and G;

x¹⁷ is selected from f and v;

x²⁴ is selected from k, l, m, r, t and v;

x²⁷ is selected from k and r;

x²⁸ is selected from a, G, q, r and s;

x³² is selected from a, G and s; and

x³⁵ is selected from d, e, q and t.

Clause 64. The D-peptidic compound of any one of clauses 57-63, wherein the D-peptidic Z domain comprises a three-helix bundle of the structural formula:

[Helix 1^{(#8-18)}]-[Linker 1^{(#19-24)}]-[Helix 2^{(#25-36)}]-[Linker 2^{(#37-40)}]-[Helix 3^{(#41-54)}]

wherein:

# denotes reference positions of amino acid residues comprised in the D-peptidic Z domain; and

Helix 3^{(#41-54)} comprises a peptidic framework sequence selected from: a)

a)
(SEQ ID NO: 58)
s⁴¹anllaeakklnda⁵⁴;

b) a sequence having 70% or more (e.g., 75% or more, 80% or more, 85% or more, or 90% or more) identity to the amino acid sequence set forth in (a); or

c) a sequence having 1 to 5 amino acid residue substitutions relative to the sequence defined in (a), wherein the 1 to 5 amino acid residue substitutions are selected from:
i) a similar amino acid residue substitution according to Table 1;
ii) a conservative amino acid residue substitution according to Table 1; and
iii) a highly conserved amino acid residue substitution according to Table 1.
   Clause 65. The D-peptidic compound of any one of clauses 57-64, wherein the D-peptidic Z domain further comprises a C-terminal peptidic framework sequence having 70% or more (e.g., 75% or more, 80% or more, 85% or more, or 90% or more) identity with the amino acid sequence:

| (SEQ ID NO: 59) | |
|---|---|
| | d³⁶dpsqsanllaeakklndaqapk⁵⁸. |

Clause 66. The D-peptidic compound of any one of clauses 57-65, wherein the D-peptidic Z domain further comprises an N-terminal peptidic framework sequence selected from: a)

a)
(SEQ ID NO: 60)
v¹dnx⁴fnx⁷e8;

wherein:
x⁴ is k, n, r or s; and
x⁷ is k or i; or

b) a sequence having 60% or more (e.g., 75% or more, 85% or more) sequence identity relative to the one or more segments defined in (a).

Clause 67. The D-peptidic compound of clause 66, wherein the N-terminal peptidic framework sequence is selected from:

| (SEQ ID NO: 61) | |
|---|---|
| | v¹dnkfnke⁸; |
| (SEQ ID NO: 62) | |
| | v¹dnnfnie⁸; |
| (SEQ ID NO: 63) | |
| | v¹dnrfnie⁸; |
| | and |
| (SEQ ID NO: 64) | |
| | v¹dnsfnie⁸. |

Clause 68. The D-peptidic compound of any one of clauses 57-67, wherein the D-peptidic Z domain comprises:

a) a sequence selected from one of compounds 978060 to 978065 (SEQ ID NOs: 36-41), 981195 to 981197 (SEQ ID NOs: 42-44), 979259 to 979262 (SEQ ID NOs: 24-27), and 979264 to 979269 (SEQ ID NOs: 28-33);

b) a sequence having 80% or more identity with the sequence defined in (a); or

c) a sequence having 1 to 10 (e.g., 1 to 6, 1 to 5, 1 to 4, 1 to 3, 1 to 2, 2 or 1) amino acid residue substitutions relative to the sequence defined in (a), wherein the 1 to 10 amino acid substitutions are selected from:
i) a similar amino acid residue substitution according to Table 1;
ii) a conservative amino acid residue substitution according to Table 1; and
iii) a highly conserved amino acid residue substitution according to Table 1.
   Clause 69. The D-peptidic compound of clause 68, wherein the D-peptidic Z domain comprises a polypeptide of one of compounds 978060 to 978065 (SEQ ID NOs: 36-41), 981195 to 981197 (SEQ ID NOs: 42-44), 979259 to 979262 (SEQ ID NOs: 24-27), and 979264 to 979269 (SEQ ID NOs: 28-33).
   Clause 70. The D-peptidic compound of any one of clauses 57-69, wherein the compound is dimeric.
   Clause 71. The D-peptidic compound of any one of clauses 57-69, wherein the compound further comprises a second D-peptidic Z domain that is homologous to the first D-peptidic Z domain.
   Clause 72. A pharmaceutical composition, comprising:

the D-peptidic compound according to any one of claims 1-72, or a pharmaceutically acceptable salt thereof; and

a pharmaceutically acceptable excipient.

The following examples are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Example 1: Engineering D-Peptidic Binders to Non-Overlapping Epitopes on PD-1

Programmed cell death protein1 (PD-1) is a highly validated therapeutic target for immune checkpoint blockade in oncology. Antagonists that block the interaction between PD-1 and its ligand PD-L1 have been shown to activate exhausted T-cells within tumors resulting in anti-tumor activity and improved patient survival in oncology. Current anti-PD-1 antibody therapeutics typically have poor tumor penetration and can elicit anti-drug antibody (ADA) responses ultimately limiting their activity in patients. D-proteins that antagonize PD-1 could overcome these limitations with their smaller size and lack of immunogenicity. Here, mirror image phage display was used to engineer bivalent D-peptidic compounds that bind to two distinct sites on the PD-1 target protein.

A prerequisite of mirror image phage display is to synthesize the D-enantiomer of the target for panning. The PD-L1-binding domain of PD-1, residues 25-167, was chemically synthesized from D-amino acids and refolded into its active tertiary structure. Briefly, D-PD-1 was first synthesized as four separate peptide fragments and then ligated using native chemical ligation. The full length product was purified using HPLC, denatured in 8M urea and refolded into its active form. Biotinylated D-PD-1 was used as target bait for panning the GA domain and Z domain phage display libraries (e.g., as described herein).

A new phage display library based on the Z domain scaffold was generated as a pVIII-fusion to M13 phage. Ten positions were selected within the Z domain for randomization using kunkel mutagenesis with trinucleotide codons representing all amino acids except cysteine (FIGS. 1A and 1B).

Phage display libraries based on the GA domain and Z domain scaffolds were generated as pVIII-fusions to M13 phage. Eleven positions within the GA domain scaffold and 10 positions within the Z domain scaffold were selected for randomization using kunkel mutagenesis with trinucleotide codons representing all amino acids except cysteine (FIGS. 1A-1B and 2A-2B). The resulting GA domain and Z domain libraries were panned against refolded D-PD-1 using mirror image phage display methods (e.g., as described herein). Briefly, 3 rounds of panning against biotinylated D-PD-1 were carried out under increasingly stringent wash conditions. After the 3^{rd} rounds, phage binders were transferred to a pIII-fusion phagemid to reduce the copy number on phage particles and an additional 2 rounds of panning were carried out. After the last round of selection on pIII individual phage clones were sequenced and analyzed for consensus motifs.

Selected variant GA domain binders yielded a preferred consensus motif containing W, S, S, S, Y, H, Y at positions 31, 37, 39, 40, 43, 44, and 47 of the GA domain, respectively (FIG. 3A; FIG. 50).

Selected variant Z domain binders yielded a preferred consensus motif containing W, A, D, F, K at positions 10, 13, 14, 17, and 27 of the ZA domain, respectively (FIG. 4A; FIG. 51).

Four representative variant GA domain sequences (FIG. 3B) (SEQ ID NOs: 32-35) and 6 representative variant Z domain sequences (FIG. 4B) (SEQ ID NOs: 36-41) were synthesized as D-peptidic compounds and their binding affinities to natural L-PD-1 were measured using SPR. For the variant GA domain compounds, compound 977296 had the highest L-PD-1 affinity with a measured equilibrium dissociation constant (K_{D}) of 625 nM (FIG. 3B). For the variant Z domain compounds, compound 978064 had the highest L-PD-1 affinity with a measured equilibrium dissociation constant (K_{D}) of 887 nM (FIG. 4B). The data confirms that both scaffolded libraries produced independent D-peptidic compounds that bind to PD-1.

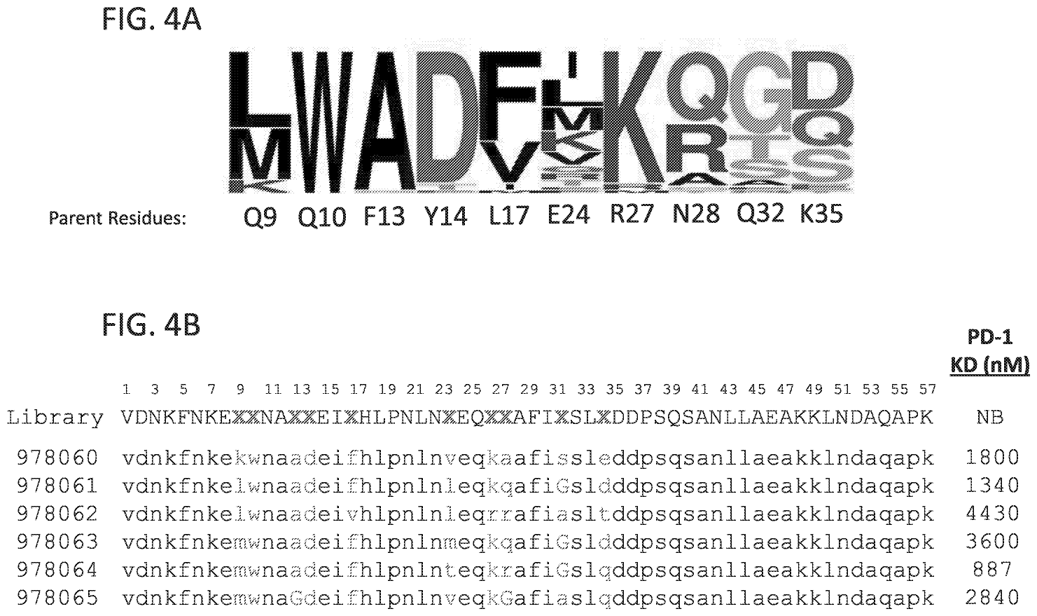

Epitope mapping by SPR was carried out to determine whether compounds 977296 and 978064 bound non-overlapping binding sites on PD-1. Here, biotinylated PD-1-Fc is captured on the SPR chip and 1 μM of 977296 is bound in the first association step in order to saturate its binding site. In a second association step, 1 μM 977296 is mixed with 1 μM 978064 and the change in steady state binding is measured. The sensorgram data displays a significant increase in response units due to 978064 binding, which is above the initial saturating level of 296 alone, indicating simultaneous and additive binding of 977296 and 978064 (see e.g., FIG. 5).

The target blocking activities of compounds 977296 and 978064 were characterized in an ELISA measuring PD-1 binding to its ligand, PD-L1. Here, PD-L1-Fc was coated overnight on a Maxisorp plate at 2 gg/mL in PBS. 2 nM biotinylated-PD-1-Fc was mixed with antagonist titrations and binding of biotinylated-PD-1-Fc to PDL1-Fc was detected with streptavidin-HRP. Compound 978064 could antagonize the interaction with PD-L1 with a measured IC₅₀ of 257 nM, although this is 250-fold weaker than the clinically approved PD-1 antagonist, nivolumab (FIG. 6). Unlike 978064, 977296 showed no detectable inhibition of PD-1 binding to PD-L1, indicating it does not bind an epitope that overlaps with the PD-L1 binding site. These data are consistent with the observation that compounds 977296 and 978064 bind to independent non-overlapping epitopes.

To further characterize the PD-1 binding sites of compounds 977296 and 978064 an X-ray crystal structure of PD-1 in complex with both compounds 977296 and 978064 was solved. Diffraction quality crystals were grown in 0.1 M Bis-Tris, pH 5.5, 0.2 M ammonium sulfate, 25% w/v PEG 3350 using the hanging drop method. The structure was solved by molecular replacement. The crystal structure of the triple complex reveals that compound 978064 directly overlaps with the PD-L1 binding site on PD-1 (FIGS. 7A and 7B), explaining the observed antagonism of 978064. Interestingly, 977296 binds PD-1 on a beta-sheet face opposite that of 978064 and distal to the PD-L1 binding site, explaining the lack of antagonism for 977296. Taken together, these data show the GA domain and Z domain libraries yielded unique D-peptidic binders to distinct binding sites on PD-1, demonstrating the utility of using two different scaffolds to target separate sites, similar to the results obtained with the VEGF-A target protein.

Structure-based affinity maturation methods were used to improve upon the PD-1 binding affinity of compound 978064. Based on the consensus sequence (FIG. 4A; FIG. 51), five residue positions (9, 24, 28, 32 and 35) displayed significant variation (i.e., residues m9, t24, r28, G32 and q35 of compound 978064). Furthermore, in the crystal structure of compound 978064 bound to PD-1 (FIG. 4E) the residues k4, f5, n6, k7 and i31 were close to the surface of PD-1, but were not included in the original library, indicating potential sites for improvement. In total, these 10 sites were selected for soft-randomization using kunkel mutagenesis (see “x” positions in FIG. 4F library). The resulting pIII phage library was panned using similar high-stringency conditions as above to find improved binders to D-PD-1. After the fifth round of selection strong consensus emerged at all sites except K4 (FIG. 4F). Three individual clones were selected to represent the consensus with variation at K4 (variants 981195, 981196 and 981197) (FIG. 4G). These were synthesized as new D-peptidic compounds, and their affinities measured by SPR to be 391 nM, 229 nM and 278 nM, respectively (FIG. 4G).

### Example 2: Bivalent D-Peptidic Antagonists of PD-1

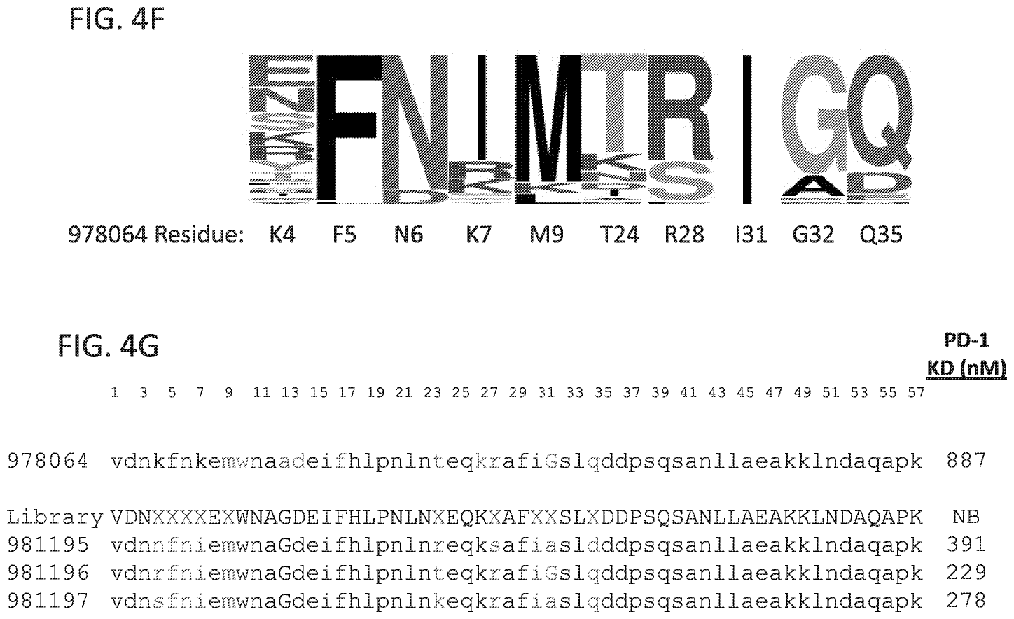

Given that D-peptidic compounds 977296 and 978064 bind PD-1 at non-overlapping binding sites and that compound 978064 directly blocks the PD-L1 binding site, we engineered a chemically linked conjugate of compounds 977296 and 978064 in order to assess the overall effect on binding and antagonistic activity. Both compounds 977296 and 978064 (FIG. 8B) were chemically synthesized with additional N-terminal cysteine residues, which were then conjugated with a series of bis-maleimide PEG linkers (e.g., PEG3, PEG6 or PEG8) (FIG. 8A). The conjugate compounds 979821, 979820, and 979450 exhibited PD-1 binding affinities of 0.29 nM, 0.37 nM and 0.59 nM, respectively, as measured by SPR (FIG. 8B). This represents >1000-fold improvement in affinity for the conjugates over the individual binder components. This is consistent with an avidity effect whereby linking the two independent binders into single heterodimer results in a molecule with higher affinity than either binder alone, a similar effect to that observed for the D-peptidic bivalent compound conjugate antagonists of VEGF-A described above. Importantly, in the PD-1 blocking ELISA, the compound conjugates 979821, 979820, and 979450 exhibited IC₅₀ values of 1.8 nM, 2.7 nM, and 1.6 nM which was similar to nivolumab with a measured IC₅₀ of 1.5 nM (FIG. 9).

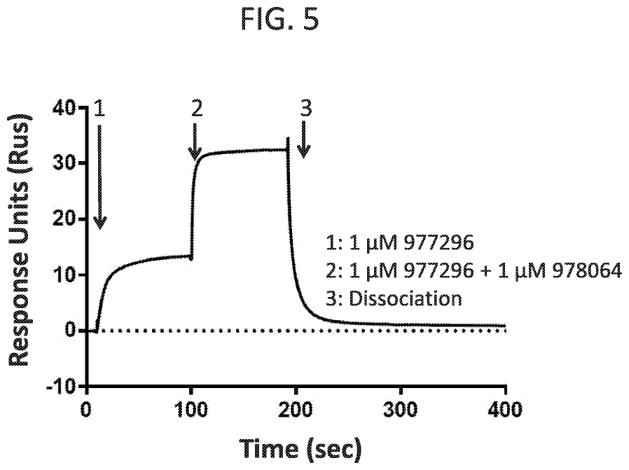

To test for biological activity, an in vitro T-cell activation assay was used to measure blockade of the PD-1/PD-L1 pathway. Here, artificial antigen presenting cells (APCs) overexpressing PD-L1 and engineered T-cells expressing PD-1 will produce luciferase upon activation of T-cell receptor (TCR) signaling. When mixed together, PD-L1 on the APCs interacts with PD-1 on T-cells and prevents TCR signaling leading to suppression of luciferase production. Upon blockade of the PD-1/PD-L1 interaction, TCR signaling is restored and an increase in luciferase production is measured. In this assay, the D-peptidic compound conjugates 979821, 979820, and 979450 exhibited IC50s for T-cell activation of 115 nM, 27 nM, and 34 nM, respectively, approaching that of Opdivo, which had a measured IC50 of 2.5 nM (FIG. 10). Taken together, these results demonstrate that bivalent D-peptidic compound antagonists of PD-1 can activate TCR signaling in a cell-based assay and may find use in therapeutic applications.

### Example 3: A Potent, Non-Immunogenic D-Protein Inhibitor of Programmed Cell Death Protein 1

A synthetic, multivalent D-protein was engineered as a molecular clasp, antagonizing PD-1 and activating T-cells while being non-immunogenic.

Chemical protein synthesis, mirror-image phage display, and structure-guided optimization were used to engineer a fully-synthetic, multivalent D-protein antagonist of programmed cell death protein 1 (PD-1) that blocks association with the PD-1 ligand (PD-L1). Peptide synthesis and native chemical ligation were utilized in constructing PD-1 in both L- and D-enantiomeric forms. Phage panning against D-PD-1 identified two separate proteins that bound non-overlapping epitopes. A co-crystal structure of this PD-1 complex facilitated the design of a multivalent D-protein that potently inhibits PD-1 binding to PD-L1, blocks PD-L1-mediated T-cell exhaustion, and restores cytokine production with activity comparable to nivolumab. In contrast to the antibody, the D-protein was non-immunogenic following repeated subcutaneous immunizations.

Main Text:

Antibodies directed against the immune checkpoint targets PD-1 and PD-L1 have demonstrated remarkable success in treating several different types of cancers (1, 2), and antagonistic antibodies to PD-1 can help overcome T-cell exhaustion and revitalize the immune system to attack tumors (3-5). However, only a small fraction of cancer patients in a subset of indications have shown durable responses after treatment with these immunotherapies (6).

D-proteins represent a therapeutic modality capable of achieving improved tumor bioavailability due to their small size and resistance to proteolysis. Being a fraction of the size of a typical antibody enables better tissue and tumor penetration, while the proteolytic stability of D-proteins protects them from degradation in the protease-rich tumor microenvironment (12, 13). Their resistance to proteases also inhibits their presentation to T cells by the major histocompatibility complex (MHC), rendering them non-immunogenic.

The total chemical synthesis and in vitro folding of human PD-1 in both its L- and D-enantiomeric forms are described herein. Based on this advance, a systematic approach was applied to developing a synthetic, multivalent 19.6 kDa D-protein that inhibits PD-1 signaling with antibody-like affinity and potency. The D-protein antagonist was described herein exhibited picomolar binding affinity for PD-1 and prevented T-cell exhaustion in cell-based assays with activity comparable to nivolumab. In contrast to nivolumab, however, the D-protein did not elicit a serum antibody response, even after repeated subcutaneous dosing in the presence of a strong adjuvant. This study supported a general framework for creating multivalent D-proteins with the ultra-high target affinity, specificity, and potency.

Total Chemical Synthesis and Refolding of PD-1

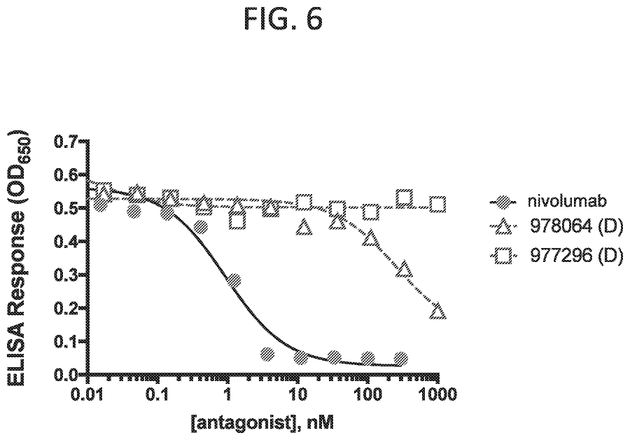

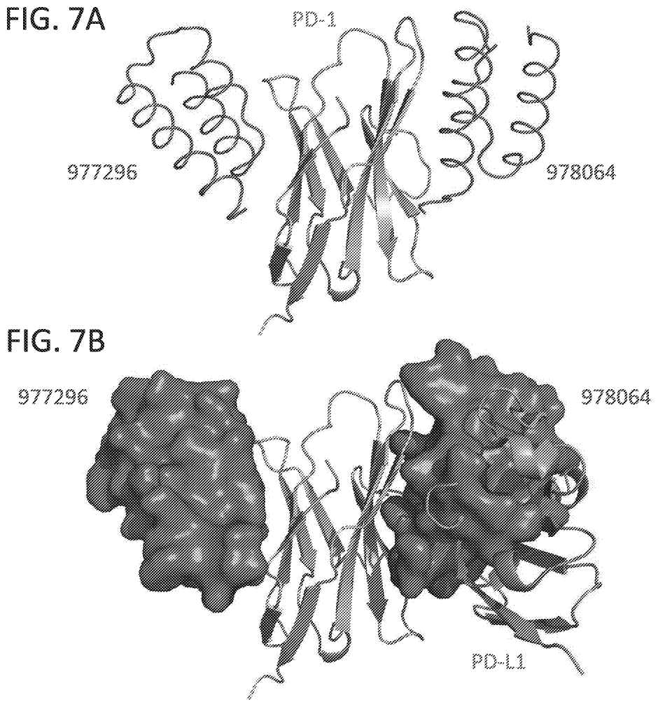

To establish a validated synthetic method for the chemical synthesis of PD-1, the L-enantiomeric form of the protein was first synthesized. Solid phase peptide synthesis (SPPS) using standard Fmoc chemistry was used to prepare each of four different linear polypeptides consisting of 1: D-His¹-to-D-Thr⁵¹, 2: D-Cys⁵²-to-D-Leu⁷⁶, 3: D-Cys⁷⁷-to-D-Leu¹²⁰, 4: D-Cys¹²¹-to D-Lys¹⁶⁷-(PEGs—Biotin) (FIG. 11). Ligations between each of the peptide-hydrazide fragments and the Cys-peptide fragments were performed sequentially until the condensation reactions reached completion, forming native peptide bonds. The ligated polypeptide was then purified by HPLC and characterized by LC-MS (FIG. 12). The purified linear PD-1 protein was then denatured and slowly refolded in an aqueous buffer to allow the native functional structure to form (methods).

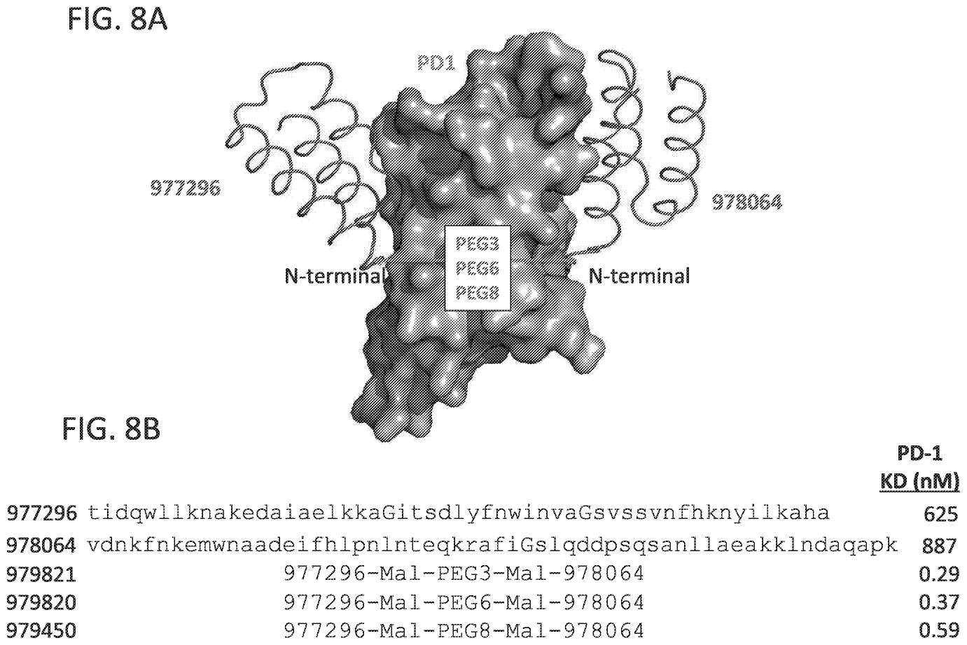

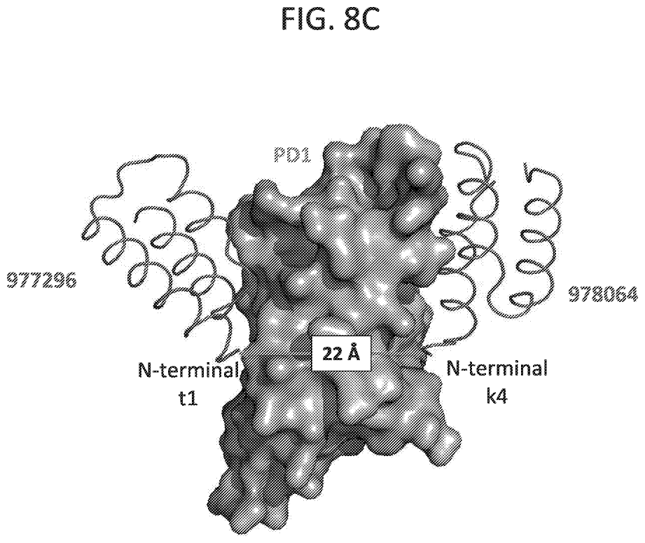

To validate that the synthetic PD-1 protein had folded into its correct tertiary structure, an ELISA assay was performed to measure binding between the refolded PD-1 and the anti-PD-1 antibody nivolumab (FIG. 13A). Dose-dependent binding was observed, with an EC₅₀ value of 0.5 nM, closely matching the reported affinity of 1.6 nM for nivolumab binding to PD-1 (19) indicating that the protein was properly folded. Binding of nivolumab to the synthetic PD-1 was also analyzed by surface plasmon resonance (SPR) and the measured K_{D} of 0.34 nM (FIG. 13B) was consistent with previously reported affinity measurements between PD-1 and nivolumab. Having established a validated method for the total chemical synthesis of PD-1, the same synthetic strategy and refolding methodology was applied using D-amino acids instead of L-amino acids to create the D-enantiomeric form of PD-1.

Multi-Scaffold Mirror-Image Protein Phage Display

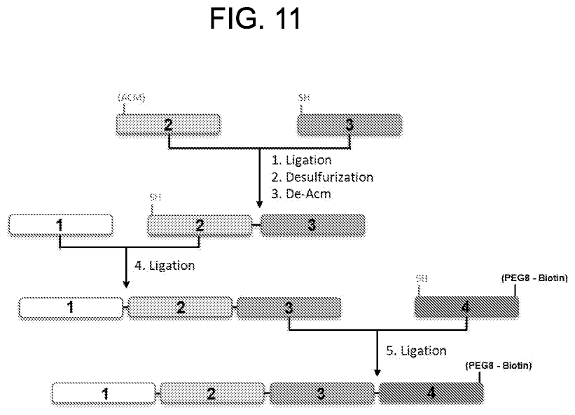

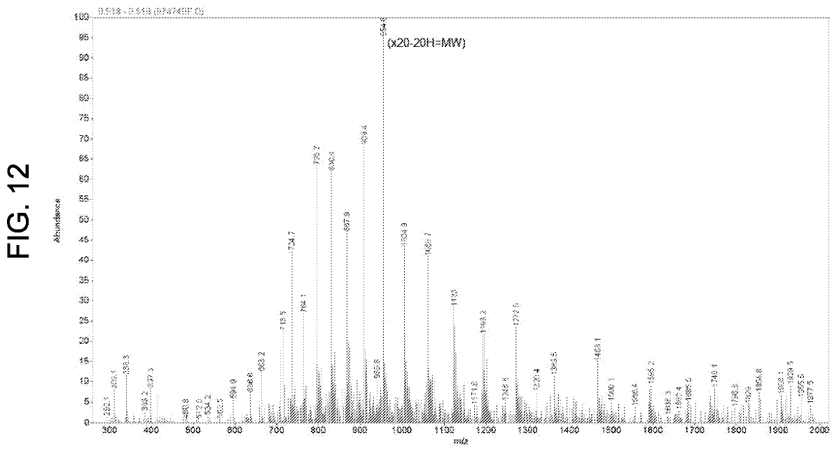

Chemical linkage of proteins binding to different sites on a therapeutic target of interest can create multivalent antagonists with ultra-high affinity (14). To discover small proteins that bind non-overlapping epitopes on PD-1, M13 phage display libraries was utilized based on two different protein scaffolds derived from different IgG Fc-binding and albumin-binding bacterial surface proteins. One phage library displayed variants of the 58-amino acid Z domain protein, while the other phage library displayed variants of the 53-amino acid GA-domain protein (FIG. 14A and FIG. 14B). Despite the fact that both of these proteins have similar 3-helix bundle structures (20, 21), libraries of these two scaffolds were used to identify binders to different epitopes on the same target (14). Each phage library was panned separately against biotinylated D-PD-1 under increasingly stringent target concentrations and wash conditions. After several rounds of selection, both libraries yielded independent, yet convergent hits which were then synthesized as the D-proteins RFX-978064 and RFX-977296 corresponding to the Z- and GA-domains respectively (FIG. 15). Binding of these D-proteins to PD-1 was measured by SPR which revealed kinetic derived equilibrium dissociation constants (K_{D}) of 904 nM for RFX-978064 and 1,507 nM for RFX-977296 (Table in FIG. 16), confirming these D-proteins retained specific binding for the natural L-enantiomeric form of PD-1.

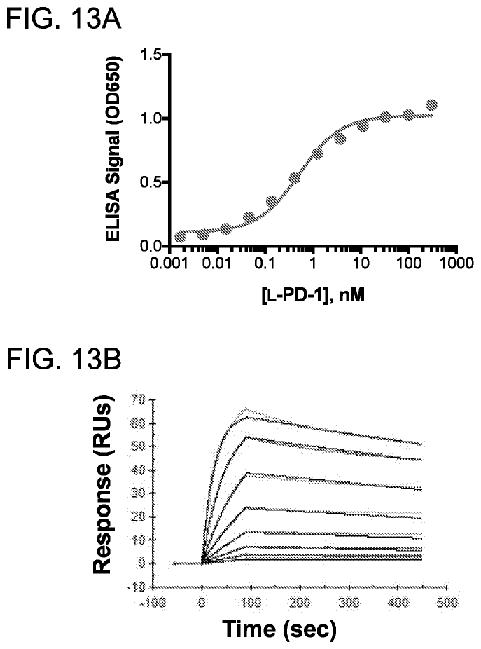

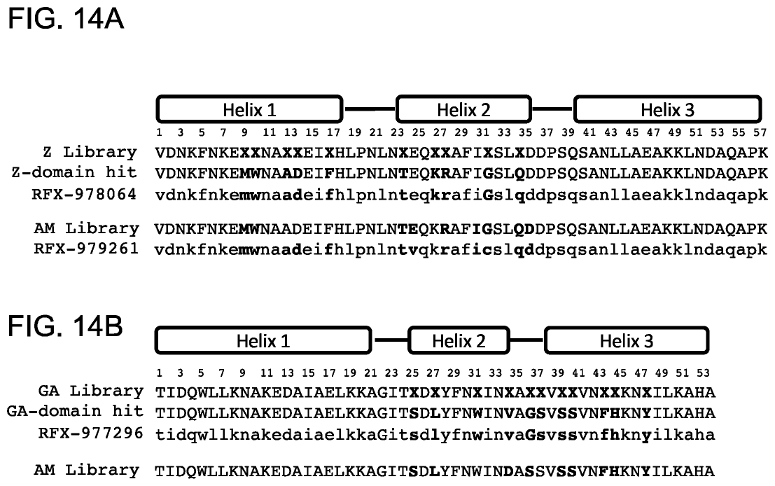

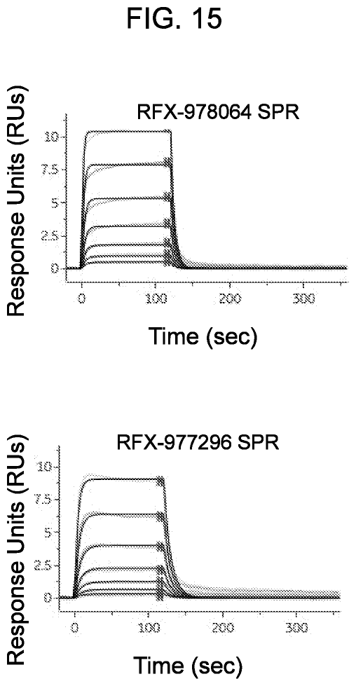

Antagonists of PD-1 signaling must block the PD-L1 ligand from interacting with PD-1 at the T-cell synapse. To assess PD-1 antagonism, a competition ELISA assay was employed measuring the ability of the D-proteins to inhibit PD-1-Fc binding to PD-L1-Fc coated on a microtiter plate. Titrations of RFX-978064 demonstrated dose-dependent inhibition of PD-1 binding to PD-L1 (IC₅₀=234 nM), whereas RFX-977296 failed to block the PD-1/PD-L1 interaction (FIG. 17 and FIG. 18). While RFX-978064 clearly showed inhibitory activity, it was much less active than nivolumab, which had an apparent IC₅₀ of 0.4 nM in this assay. To determine whether RFX-978064 binds to a different epitope than RFX-977296, an epitope mapping experiment was performed using SPR. Here, 1 μM of RFX-977296 was first bound to PD-1-Fc on the chip, followed by an equimolar mixture of 1 μM of RFX-977296 and 1 μM of RFX-978064. The SPR sensorgram showed additive binding with similar amplitudes for RFX-977296 and RFX-978064, indicating these two molecules interact with non-overlapping epitopes on PD-1 (FIG. 19).

Structure-Guided Affinity Maturation of RFX-978064 and RFX-977296

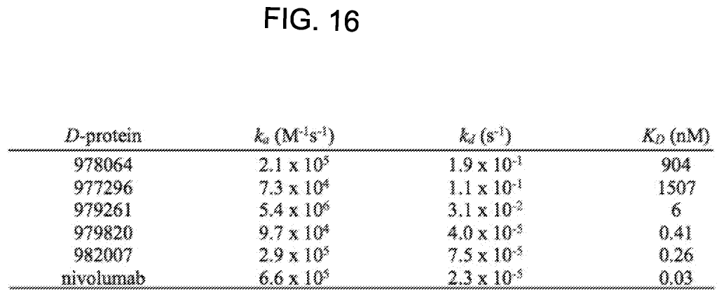

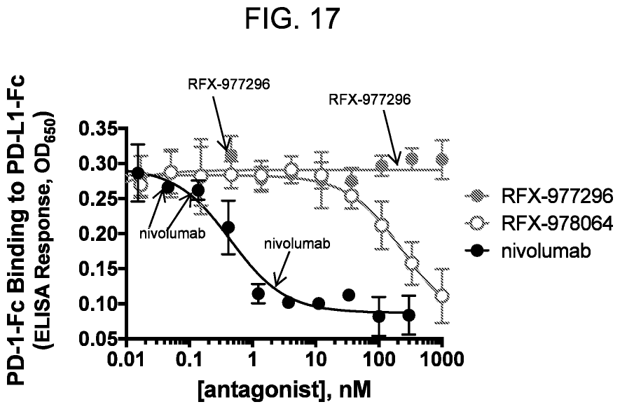

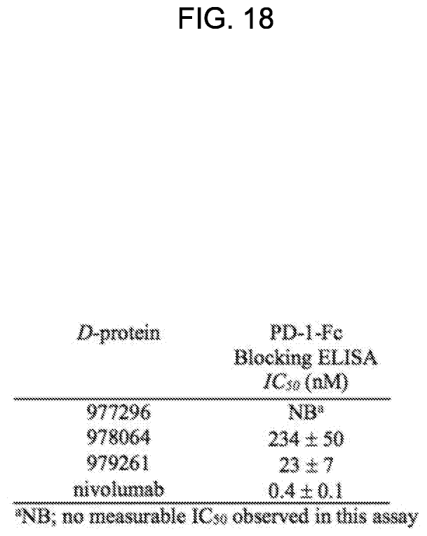

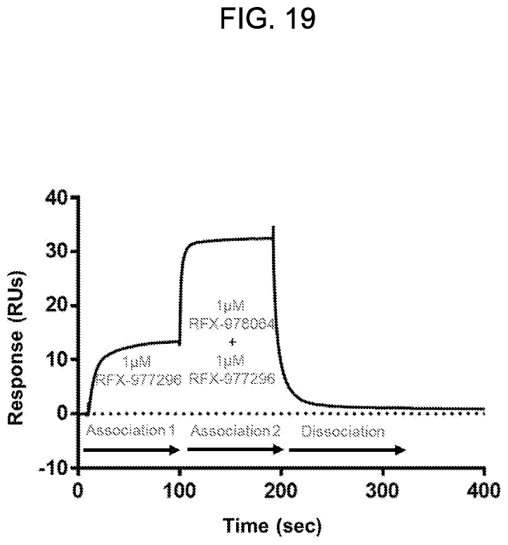

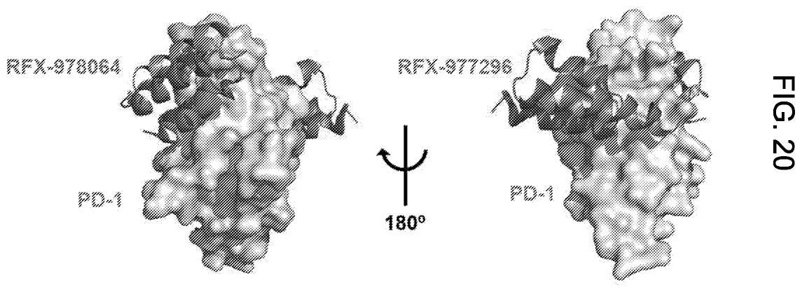

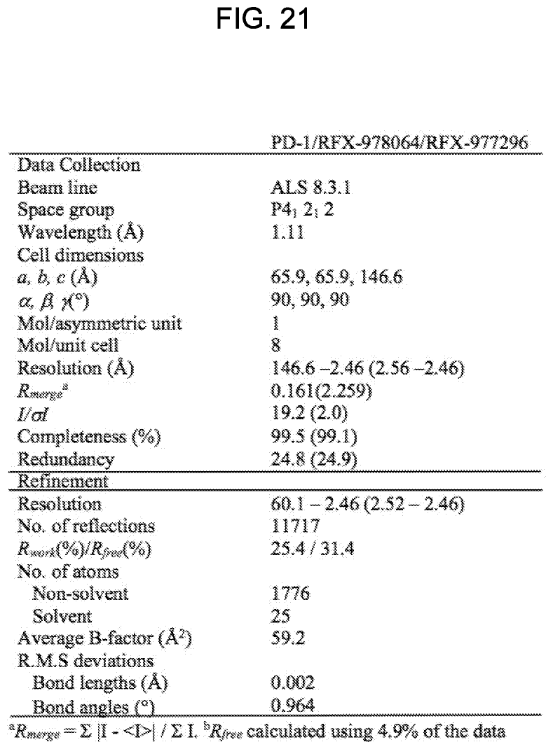

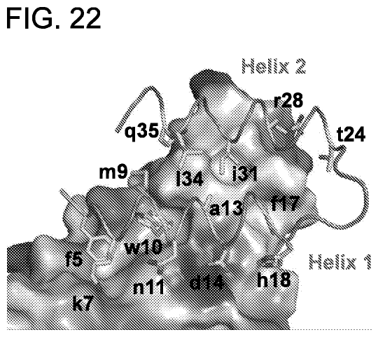

To guide further optimization of the D-proteins, an x-ray crystal structure of PD-1 simultaneously bound by both RFX-978064 and RFX-977296 was solved to a resolution of 2.46 Å (FIG. 20 and FIG. 21). The D-protein RFX-978064 binds PD-1 using a network of hydrophobic contacts (f5, w10, a13, f17, i31, and 134) as well as several polar (n11, d14, t24, and q35) and basic residues (k7, h18, r28) to interact with ˜770 Å² surface area on PD-1 (FIG. 22). An overlay of the structure with a previously solved co-crystal structure of PD-1 and PD-L1 ((22), FIG. 23A and FIG. 23B) highlights the direct overlap of the RFX-978064 and PD-L1 binding sites, in agreement with the competition observed in our ELISA results (FIG. 17). Interestingly, a conserved D-tryptophan (w10) in RFX-978064 is buried in a hydrophobic pocket of PD-1 (FIG. 22), mimicking the interaction formed by Tyrosine-123 of PD-L1 when bound to PD-1 (FIG. 24). In contrast, RFX-977296 binds a smaller epitope surface on the opposite face of the PD-1/PD-L1 interaction site (FIG. 23B), primarily utilizing hydrophobic residues (w31, v34, a35, f43, h44, and y47) in addition to a polar patch of three serines (s³⁷, s³⁹, and s⁴⁰) to interact with 550 Å² of surface area (FIG. 25). This is consistent with the observation that RFX-977296 does not block binding of PD-1 to PD-L1 (FIG. 17).

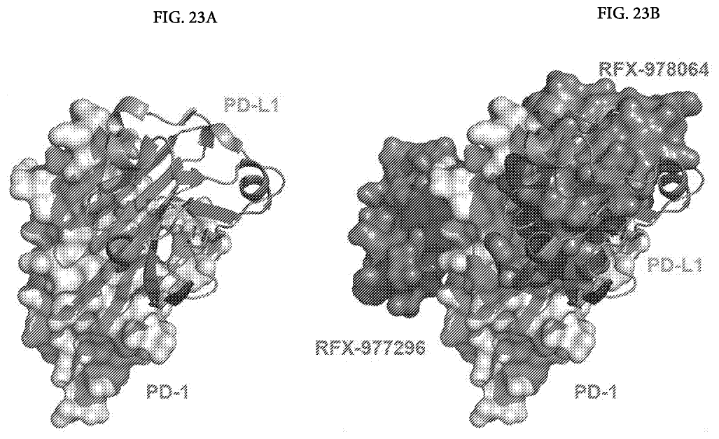

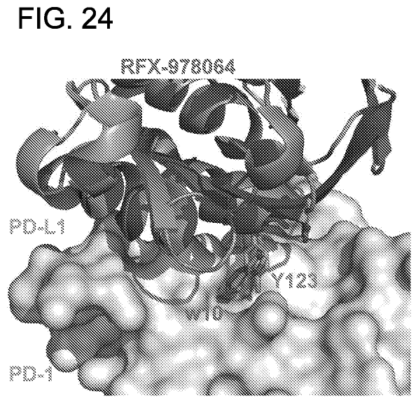

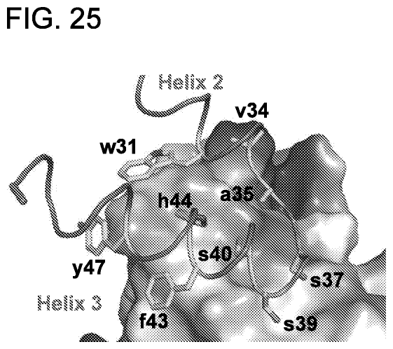

Based on the structural characterization of the RFX-978064 and RFX-977296 paratopes, soft randomization phage display libraries were designed to improve their binding affinities to PD-1. Because the interfacial residues found in Helix 2 of RFX-978064 were less conserved than Helix 1 after the initial panning, these seven residues were targeted during our affinity maturation efforts (FIG. 26). Kunkel mutagenesis was used to simultaneously randomize each with the NNC degenerate codon representing 15 possible amino acids. After an additional four rounds of panning under increasingly stringent conditions, a strong consensus motif emerged containing a G32C mutation (FIG. 14A). A cysteine mutation at this position suggests the formation of an intermolecular disulfide bond, effectively creating dimeric binders to PD-1. In support of this, the variant RFX-979261 was synthesized as a D-protein and chemically oxidized to ensure the formation of the disulfide bond (FIG. 14A). Using SPR, RFX-979261 exhibited a binding affinity of 6.0 nM, representing a ˜150-fold improvement over the parent molecule (FIG. 27 and FIG. 16). Additionally, RFX-979261 exhibited an improved IC₅₀ of 23 nM in the PD-1-Fc blocking ELISA, a ˜10-fold increase over RFX-978064 (FIG. 28 and FIG. 18).

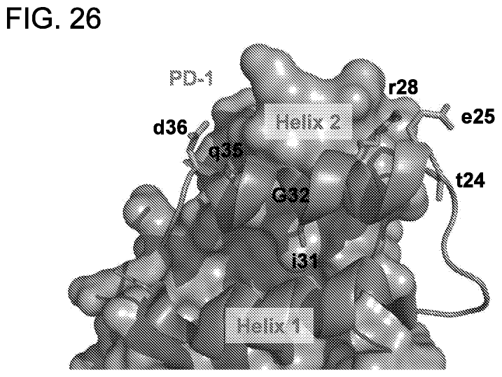

A similar soft randomization approach was employed in creating an affinity maturation library based on RFX-977296. Here, Kunkel mutagenesis was applied to nine residues including the Helix 2-loop-Helix 3 motif interacting with PD-1 (FIG. 29). However, in contrast to RFX-978064, no significant improvements in binding affinity were achieved for RFX-977296.

Design and Chemical Synthesis of Multivalent D-Protein PD-1 Inhibitors

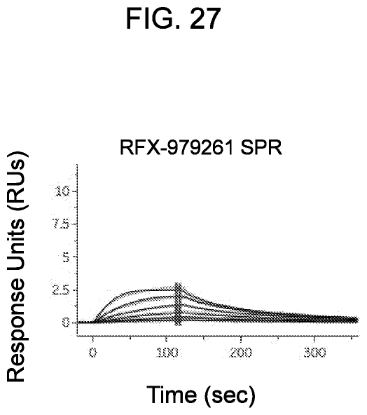

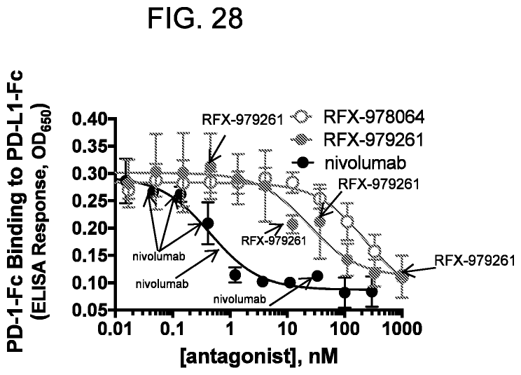

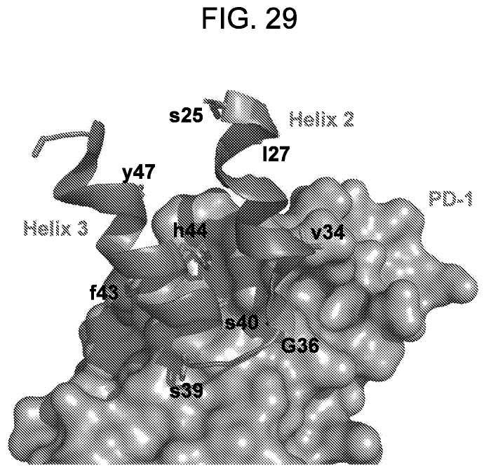

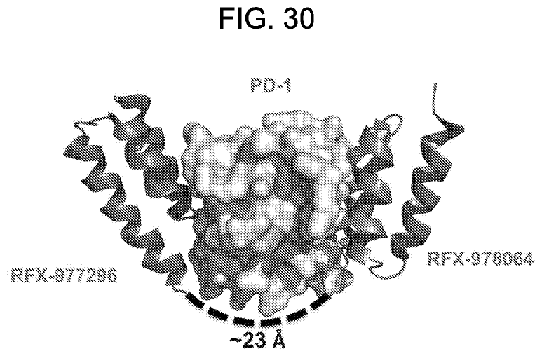

To further enhance the affinity and potency of the monomeric D-protein binders, they were chemically linked together to form a heterodimeric PD-1 clasp. The crystal structure revealed the N-termini of RFX-978064 and RFX-977296 were ˜23 Å apart (FIG. 30) and, therefore, amenable to covalent chemical linkage. The two D-proteins RFX-978064 and RFX-977296 were prepared by chemical synthesis with an additional D-cys-D-ala dipeptide on the N-termini to provide a reactive thiol group for maleimide-PEG conjugation (FIG. 31). Following synthesis, they were reacted with a bis-maleimide PEG₆ moiety to form RFX-979820, a multivalent heterodimeric D-protein which functions as a molecular clasp around PD-1 (FIG. 32). RFX-979820 was characterized by LC/MS spectra following chemical synthesis and purification Remarkably, SPR titrations revealed a K_{D} of 410 μM, representing a >2,000-fold increase in the affinity for PD-1 relative to either of the unlinked monomeric species (FIG. 33 and FIG. 16).

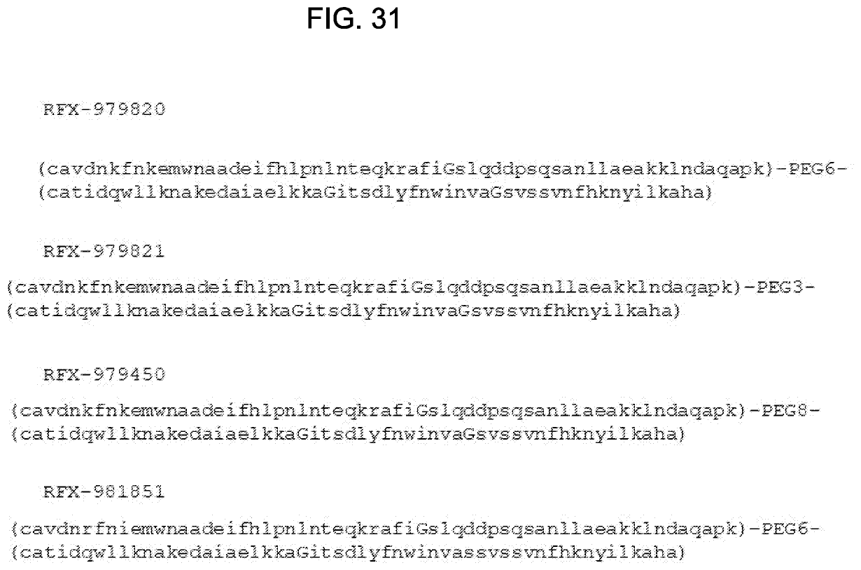

Expanding on the observed avidity effect for RFX-979820, we next linked RFX-979261 with RFX-977296 to generate a trimeric PD-1 clasp (FIG. 34). To avoid reaction with the disulfide-forming cysteine in RFX-979261, a click chemistry strategy was used instead of the maleimide-based linker. One equivalent of monomeric RFX-979261 was first reacted with PEG₃-propargylglycine to create a clickable alkyne handle in addition to the free thiol from c32. This was then reacted with a 5-Npys protected RFX-979261 intermediate to form a disulfide-linked RFX-979261 homodimer containing a PEG₃ alkyne. In parallel, one equivalent of RFX-977296 was prepared with a PEG₃-azide to form the orthogonal reactive group. In the final conjugation step, the RFX-979261 homodimer was linked to the RFX-977296 monomer using the Cu-catalyzed regioselective click reaction, yielding the 19.6 kDa trimeric D-protein RFX-982007 (FIG. 35). RFX-982007 was characterized by LC/MS spectra following chemical synthesis and purification. SPR titrations of RFX-982007 against PD-1 demonstrated an ultra-high binding affinity with a K_{D} measurement of 260 μM, within ˜8-fold of nivolumab (K_{D}=30 μM) (FIG. 33 and FIG. 16).

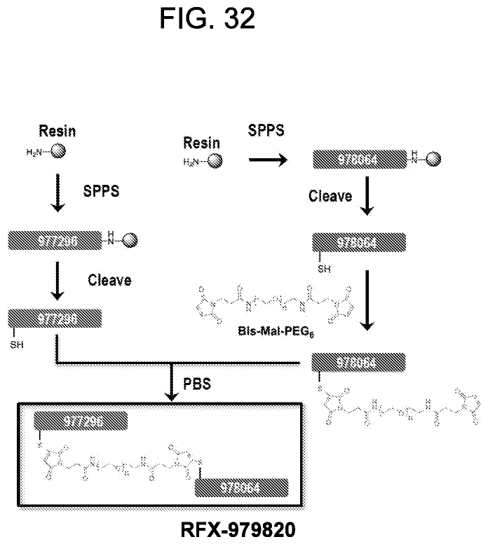

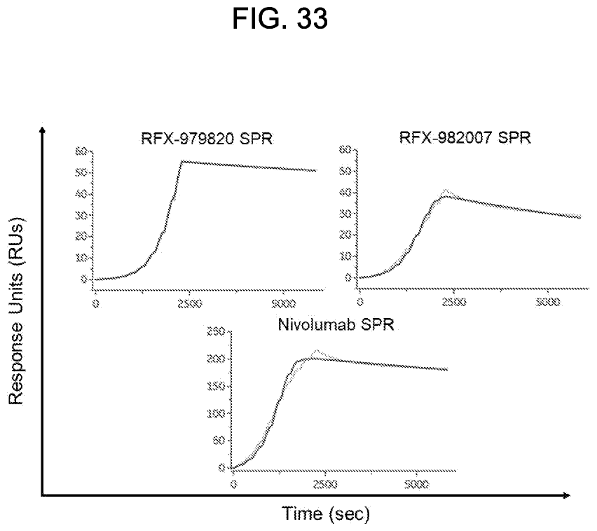

The high binding affinity achieved with RFX-982007 is consistent with a multivalent interaction enabled by the chemical linkage of the individual D-protein monomers into a trimer. To characterize the blocking potential of the high-affinity multivalent D-protein antagonists, an ELISA was utilized to measure the inhibition of PD-1-Fc binding to plate-coated nivolumab. In this assay, titrations of RFX-979820 and RFX-982007 exhibited IC₅₀ values of 830 μM and 300 μM, respectively (FIG. 36 and FIG. 37). RFX-982007 exhibited strong inhibition within 2-fold of nivolumab (IC₅₀=160 μM). As a result, the PD-L1 blocking proficiency of our synthetic clasp rivals that of approved antibody-based therapeutics like nivolumab.

A D-Protein PD-1 Clasp Prevents T-Cell Exhaustion In Vitro and is Non-Immunogenic

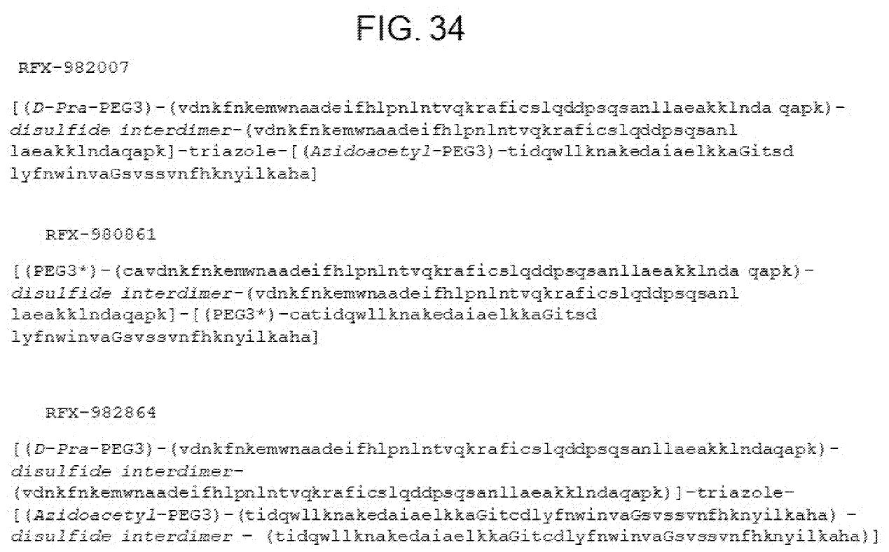

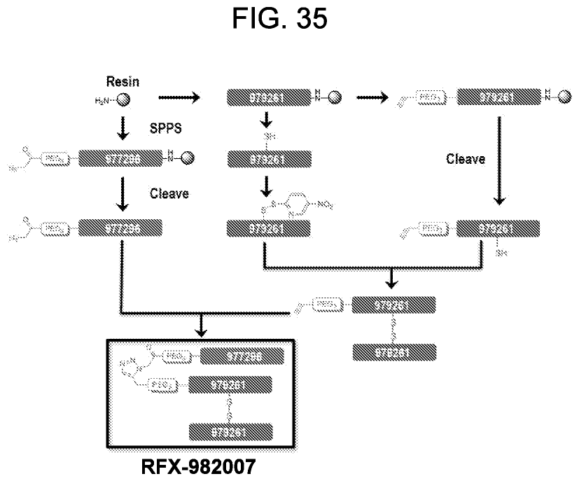

To characterize the therapeutic potential of our D-protein PD-1 clasps, their ability to block PD-1 and prevent PD-L1 mediated T-cell exhaustion was investigated in the context of an in vitro cell-based assay. To directly assess the status of T-cell receptor (TCR) signaling, a Jurkat T-cell reporter/APC co-culture assay was employed to mimic PD-1/PD-L1-induced suppression of TCR activation (methods). Here, direct PD-1 antagonism results in activation of TCR signaling and increased luciferase expression from the NFAT-driven response element. While the RFX-979261 homodimer did not show any measurable activity in the concentrations tested, both RFX-979820 and RFX-982007 exhibited dose-dependent blocking of PD-1 and activation of TCR signaling with EC₅₀ values of 26.3 nM and 4.6 nM, respectively (FIG. 38 and FIG. 39). Importantly, RFX-982007 was 6-fold more potent than RFX-979820 and within 2-fold of nivolumab, which exhibited an EC₅₀ of 2.7 nM in this assay.

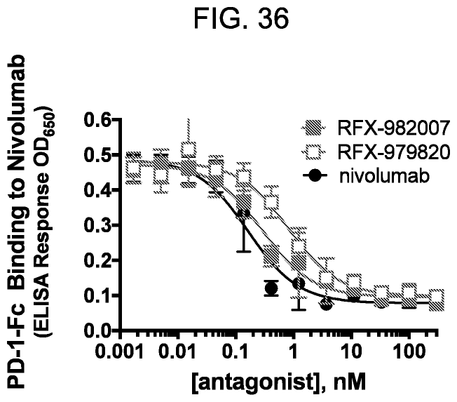

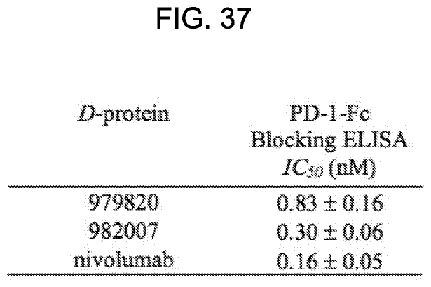

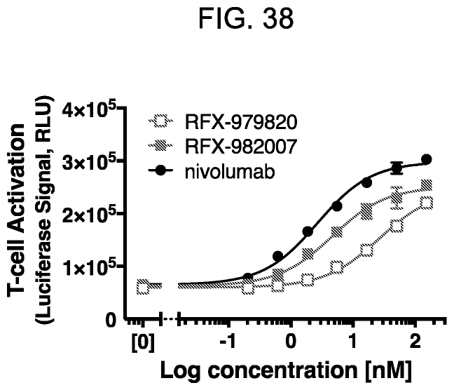

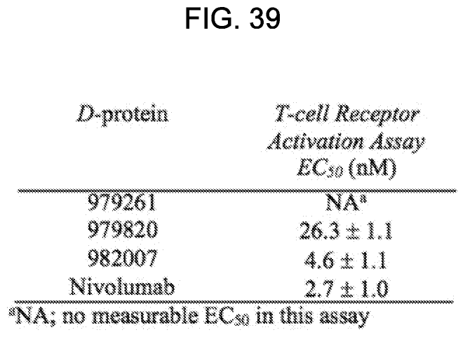

Given RFX-982007 was able to activate TCR signaling similar to nivolumab, its ability was further tested to enhance cytokine production during CMV antigen recall. In this assay, primary human PBMCs from a CMV-positive donor are challenged with isolated CMV antigens and IL-2 to induce T-cell proliferation and production of the inflammatory cytokines TNF-α and INF-y. However, these responses are suppressed in the assay due to the exhausted PD-1⁺ phenotype of CMV-specific T-cell clones (19), and the presence of a PD-1 antagonist can stimulate T-cell proliferation and cytokine production. Titration of RFX-982007 exhibited a dose-dependent increase in the proliferation of CD8⁺ and CD4⁺ T-cells (FIG. 40 and FIG. 41) and robust production of both TNF-α and INF-y cytokines (FIG. 42 and FIG. 43), reaching maximal cytokine production levels similar to nivolumab. Taken together, these results demonstrate the trimeric D-protein RFX-982007, has antibody-like PD-1 blocking activity and prevents PD-L1-mediated T-cell exhaustion in settings of TCR activation, T-cell proliferation, and cytokine production.

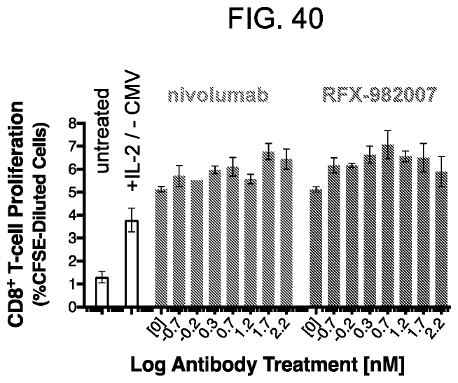

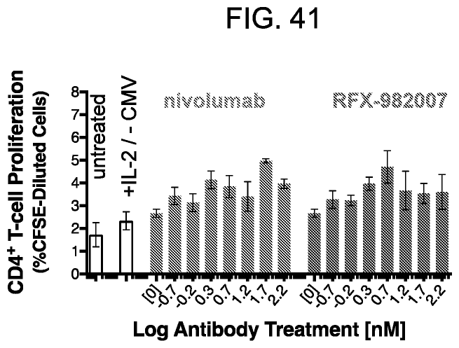

To demonstrate the non-immunogenic potential of RFX-982007, a mouse immunization study was performed to compare RFX-982007 head-to-head with nivolumab in a setting where both molecules are foreign antigens. Here, mice were repeatedly injected subcutaneously with either RFX-982007 or nivolumab emulsified in a strong adjuvant to provide immune stimulation. Immunization with nivolumab generated strong serum IgG titers against the antigen as early as Day 21, and saturated by Day 42 as determined by an ELISA to detect anti-nivolumab murine IgG (FIG. 44A). In contrast, RFX-982007 was able to avoid the humoral antibody response over the entire course of the immunization study (FIG. 44B). Thus, despite both agents being completely foreign protein-based antigens, only nivolumab elicited a strong anti-drug antibody response, highlighting the differentiation of RFX-982007 over monoclonal antibodies with respect to its absence of immunogenicity.

Discussion

The PD-1/PD-L1 immune checkpoint axis is highly validated with three anti-PD-1 antibodies (nivolumab, pembrolizumab, and cemiplimab) and three anti-PD-L1 antibodies (atezolizumab, avelumab, and durvalumab) currently approved for use in multiple oncology indications (23-28). However, there is little clinical differentiation between the antibodies, and all are susceptible to the liabilities associated with poor tissue and tumor penetration, long periods of drug exposure, and accumulation of anti-drug antibodies over time, ultimately hindering their efficacy (29, 7-10). Furthermore, efforts to develop small, non-antibody antagonists to overcome these challenges have struggled to demonstrate target binding affinities and potencies comparable to antibodies. For example, CA-170 is the first small molecule targeting PD-L1 to enter a Phase I clinical trial (30), but recent reports have shown this compound only marginally dissociates the PD-1/PD-L1 complex in vitro with IC₅₀ values of 5-10 mM (31). Likewise, the PD-1/PD-L1 antagonist AUNP-12 is a 29-amino acid L-peptide that binds PD-L1 with a K_{D} in the low millimolar range, and is therefore unlikely to show efficacy given its weak binding affinity and susceptibility to proteolytic degradation. Generally, it is thought that the poor activity associated with small molecule and peptide antagonists results from the difficulty of these classes of molecules to effectively target the flat, dynamic, and hydrophobic PD-1/PD-L1 interface (32, 33).

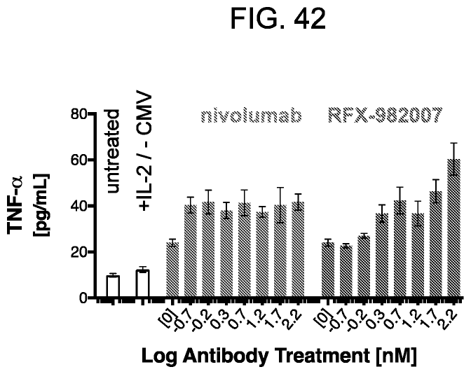

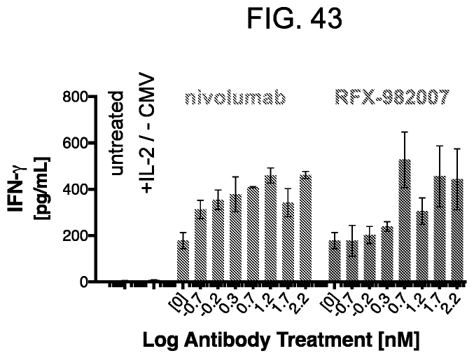

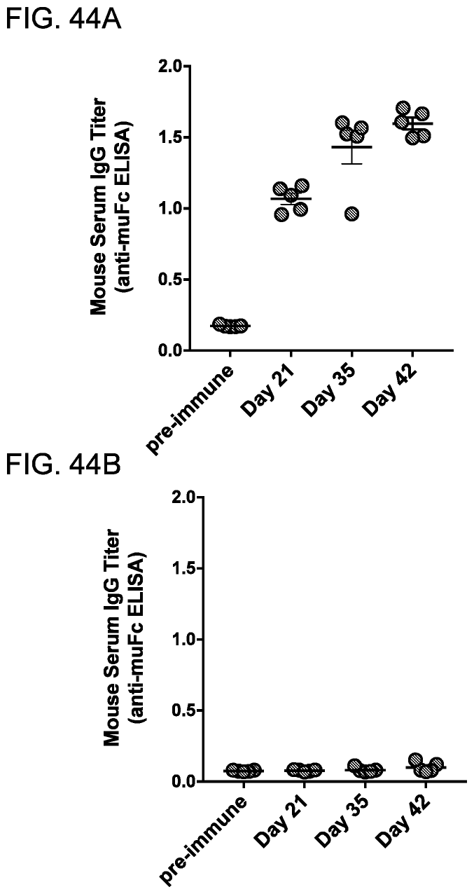

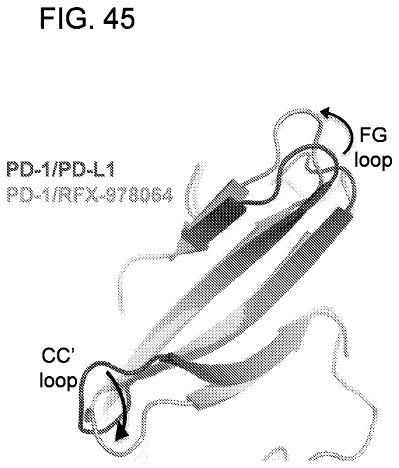

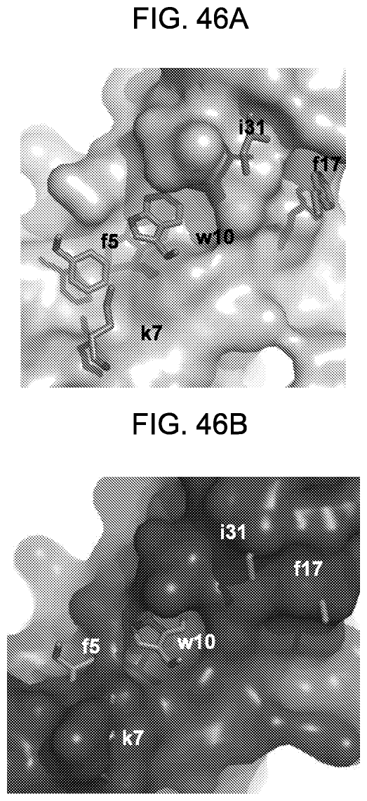

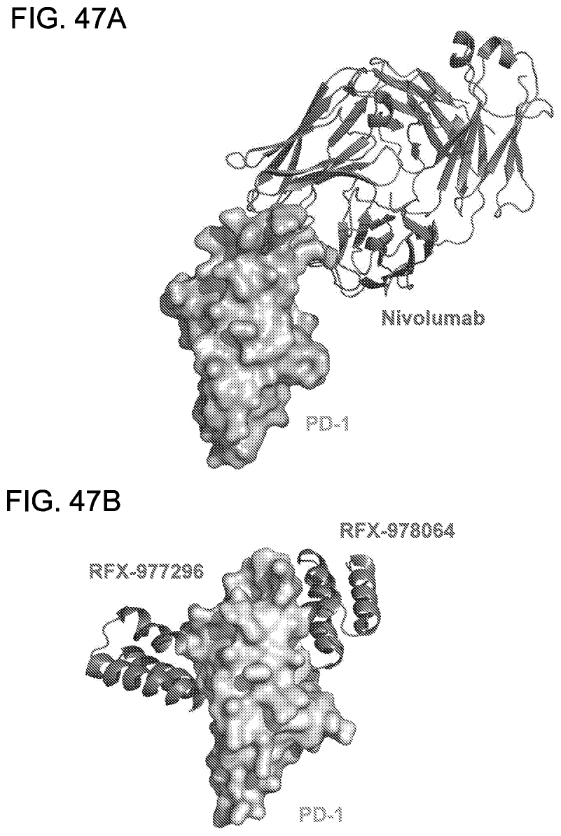

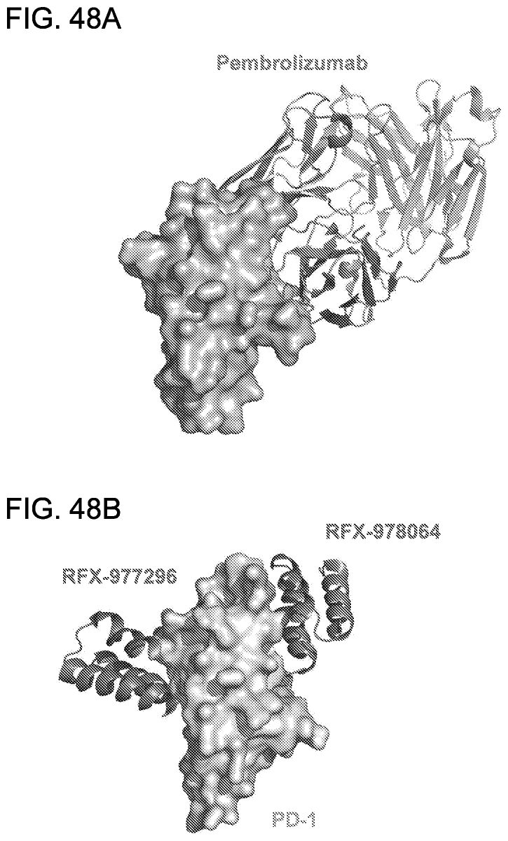

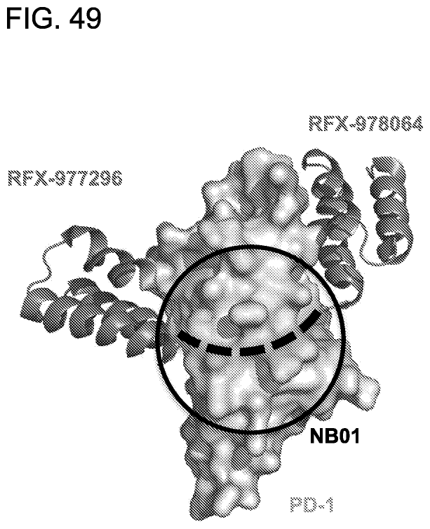

The use of mirror-image phage display is reported herein to create RFX-982007, a highly-differentiated, non-antibody antagonist of PD-1. This 19.6 kDa multivalent D-protein potently blocks association of PD-L1 with PD-1 and exhibits antibody-like activity in cell-based assays. Structural characterization of the independent D-protein domains that comprise RFX-982007 illustrate a molecular clasp mechanism, whereby dual binding to both the PD-L1 interaction site as well as a distal, non-competitive epitope creates a high-avidity PD-1 antagonist (FIG. 23B). Interestingly, loop rearrangements in RFX-978064-bound PD-1 relative to the PD-Li-bound structure (FIG. 45) form new cavities that accommodate four hydrophobic sidechains of RFX-978064 (f5, aliphatic chain of k7, f17 and i31), all of which are occluded in the PD-Li-bound structure (FIG. 46A and FIG. 46B). The RFX-978064 site is also targeted by approved anti-PD-1 antibodies nivolumab (FIG. 47A and FIG. 47B) and pembrolizumab (FIG. 48A and FIG. 48B) (34), while RFX-977296 binds an epitope away from the PD-L1 interaction site. This site is also targeted by the antibody NB01a, which is proposed to block PD-1 association with CD28 and cooperate with PD-L1 antagonism to relieve T-cell exhaustion (FIG. 49) (35, 36). Ultimately, conjugation of RFX-979261 (the homodimeric variant of RFX-978064) to RFX-977296 yielded RFX-982007, a multivalent PD-1 antagonist with a binding affinity of 260 μM, comparable to that of nivolumab (FIG. 16) (19). This significant improvement over published non-antibody antagonists is attributed to the multivalent nature of the interaction, comprising a total surface area of ˜1300 Å², larger than the contact areas for either nivolumab (˜700 Å²) or pembrolizumab (˜1000 Å²) alone. Together, these features explain how RFX-982007 can prevent PD-L1-mediated T-cell exhaustion by restoring TCR signaling and stimulating cytokine production similar to nivolumab (FIG. 38-FIG. 43).

The multivalent D-protein PD-1 clasp as described herein is an example of extending mirror-image phage display technology for the development of novel, non-antibody immune checkpoint inhibitors with the unique properties of being non-immunogenic and resistant to proteolytic degradation. Moreover, having a short circulating half-life can decrease drug exposure times and help facilitate alternative dosing strategies.

Interestingly, recent clinical evidence shows that dual blockade of VEGF-A and the PD-1/PD-L1 axis is a promising immunotherapy combination strategy for the treatment of non-small cell lung cancer, hepatocellular carcinoma, and metastatic renal cell carcinoma (39, 40). Inhibition of VEGF-A increases infiltration of tumor-reactive CD8⁺ T cells while decreasing infiltration of CD4⁺ T_{reg} cells (41). A combination of D-protein antagonists targeting both PD-1 and VEGF-A provides a highly-differentiated, alternative therapeutic modality for treating these serious diseases.

### Materials and Methods

Protein Synthesis Reagents

Fmoc-D-amino acids were purchased from Chengdu Zhengyuan Company, Ltd. and Chengdu Chengnuo New-Tech Company, Ltd. Fmoc-D-Ile-OH was purchased from ChemImpex International, Inc. Fmoc-D-propargylglycine (Fmoc-D-Pra-OH) was purchased from Haiyu Biochem. MBHA Resin was purchased from Sunresin New Materials Co. Ltd., Xian. Rink Amide linker was purchased from Chengdu Tachem Company, Ltd. Chloro-(2-Cl)-trityl-resin was purchased from Tianjin Nankai Hecheng Science and Technology Company, Ltd. Fmoc-NH₂(PEG)n-COOH and other PEG linkers were purchased from Biomatrik Inc. 2-Azidoacetic acid was purchased from Amatek Scientific Company Ltd. Sodium ascorbate was purchased from TCI (Shanghai) Ltd. Copper sulfate pentahydrate (CuSO₄.5H₂O) was purchased from Energy Chemical.

D-PD-1 Synthesis and Refolding

The D-PD-1 polypeptide chain was chemically synthesized with a 6×His tag and a TEV cleavage site on the N-terminus and a biotinylated PEGs linker on the C-terminus using solid phase peptide synthesis (SPPS) and native chemical ligation, and then folded using methods adapted from our previous work (14). The full construct that was synthesized is as follows: hhhhhhssgvdlgtenlyfqsaldspdrpwnpptfspallvvtegdnatftcsfsntsesfvlnwyrmspsnqtdklaafpedrsqpgqds rfrvtqlpngrdfhmsvvrarrndsgtylcgaislapkaqikeslraelrvterraevptahpspsprpagqfk-PEGs-biotin. Individual peptide fragments corresponding to 1: D-His¹-to-D-Thr⁵¹, 2: D-Cys⁵²-to-D-Leu⁷⁶, 3: D-Cys⁷⁷-to-D-Leu¹²⁰, 4: D-Cys¹²¹-to D-Lys¹⁶⁷- (PEGs—Biotin) were synthesized using standard Fmoc chemistry protocols for stepwise SPPS (FIG. 1). Fragments 1-3 were synthesized on hydrazine resin and fragment 4 was synthesized from pre-loaded Wang Resin. Briefly, preloaded Fmoc-aminoacyl-Wang Resin was initially swelled with DMF (10 mL/g) for 1 hour, then treated with 20% piperidine/DMF (30 min) to remove the Fmoc group and washed again with DMF (5 times). Fmoc-D-amino acid residues were coupled by addition of a pre-activated solution of 3 equivalents each of protected amino acid (0.4 M in DMF), diisopropylcarbodiimide (DIC), and hydroxybenzotriazole (HOBt) to the resin. After 1-2 h, the ninhydrin test showed the reaction was completed and the resin was washed with DMF (3 times). To remove the Fmoc group, piperidine (20% in DMF) was added to the resin for 30 min. After removal of the final Fmoc group, the resin was rinsed with DMF (3 times) and MeOH (2 times), dried under vacuum, then taken up in 85% TFA, 5% thioanisole, 5% EDT, 2.5% phenol and 2.5% water for deprotection and cleavage. After 3 h, the suspension was filtered, and the resin was washed with TFA and the filtrates were combined. The crude peptides were precipitated with cold ether, pelleted by centrifugation, and washed with cold ether 2 times before drying under vacuum. Crude peptide residue was dissolved in water, purified by preparative reverse phase HPLC and analyzed by HPLC and MS.

Ligations between D-peptide-hydrazide fragments and D-Cys-peptide fragments were performed as follows: D-peptide-hydrazide was dissolved in Buffer A (0.2M sodium phosphate containing 6 M GnHCl, pH 3.0), cooled to −15° C. in an ice-salt bath, and gently stirred by magnetic stirrer. NaNO₂ (7 equivalents) was added and the solution stirred for 20 min to oxidize the D-peptide-hydrazide to the D-peptide-azide. A solution of 4-mercaptophenyl acetic acid (MPAA) (50 eq) dissolved in Buffer B (0.2M sodium phosphate containing 6 M GnHCl, pH 7.0) was quickly added to the solution containing the newly-formed D-Peptide-azide (equal volume) to eliminate excess NaNO₂ and to convert the D-peptide-azide to the D-peptide-MPAA thioester. Then a solution of D-Cys-peptide in Buffer B (equal volume) was added to the solution containing the newly formed peptide-MPAA thioester. The reaction mixture was adjusted to pH 7 with NaOH to initiate overnight native chemical ligation. Reaction progress was monitored by analytic RP-HPLC until completion, then treated by TCEP before HPLC purification.

The ligated peptide product was then dissolved to 4 mg/mL in a desulfurization buffer (0.2M sodium phosphate containing 6 M GnHCl and 0.5 M TCEP, pH=6.5) and then tBuSH and VA-044 were added to the solution and stirred at room temperature overnight. The progress of the reaction was monitored by analytic RP-HPLC until completion.

Purification of the ligated peptide product was performed on a CXTHLC6000/Hanbon NU3000 prep system on YMC C4 silica with columns of dimension 20.0×250 mm. Crude peptides were loaded onto the prep column and eluted at a flow rate of 20 mL per minute with a shallow gradient of increasing concentrations of solvent B (0.1% TFA in 80% acetonitrile/20% water) in solvent A (0.1% TFA in water). Fractions containing the purified target peptide were identified by analytical LC-MS, combined, and lyophilized.

The final linear D-PD-1 polypeptide was folded at pH 7.5 in aqueous HEPES (25 mM) containing NaCl (25 mM), KCl (1 mM), L-Arginine (0.5M), GSH (1 mM), GSSG (9 mM), and 5% glycerol and stirred for 3 days at 4° C. to reach completion. The protein was then dialyzed 3 times against 20 volumes of dialysis buffer (25 mM HEPES, 500 mM NaCl, 5% glycerol pH=7.4) for 3 days at 4° C.

Phage Display Libraries and Panning

Naïve GA- and Z domain scaffold libraries were constructed as fusions to the N-terminal gene 8 major coat protein by previously described methods (42). Randomization of desired library positions (FIGS. 12 and 13A-13B) was performed using Kunkel mutagenesis (43) with trinucleotide oligos allowing incorporation of all natural amino acids except cysteine. The resulting libraries contained >10¹⁰ unique members. For affinity maturation libraries, Kunkel mutagenesis was performed on RFX-977296 or RFX-978064 parent sequences using targeted NNC or soft-randomization oligos, respectively. Positions targeted for affinity maturation are highlighted in FIGS. 12 and 13A-13B.

All phage selections were executed according to previously established protocols (14). Briefly, selections with the peptide libraries were performed using biotinylated D-PD-1 captured with streptavidin-coated magnetic beads (Promega). Initially, three rounds of selection were completed with decreasing amounts of D-PD-1 (2.0 μM, 1.0 μM, and 0.5 μM). The phage pools were then transferred to a N-terminal gene 3 minor coat protein display vector and subjected to an additional three rounds of panning with decreasing amounts of D-PD-1 (200 nM, 100 nM, and 50 nM) and increased wash times. Individual phage clones were then sent in for sequencing analysis.

Synthesis of Monomeric D-Proteins RFX-977296, RFX-978064, and RFX-979261

The polypeptide chains of the monomeric D-proteins RFX-977296 and RFX-978064 as well as the affinity-matured RFX-979261 (FIG. 14A) were prepared manually by Fmoc chemistry stepwise SPPS on Rink Amide MBHA Resin. Side-chain protection for amino acids was as follows: D-Arg(Pbf), D-Asp(OtBu), D-Glu(OtBu), D-Asn(Trt), D-Gln(Trt), D-Ser(tBu), D-Thr(tBu), D-Tyr(tBu), D-His(Trt), D-Lys(Boc), D-Trp(Boc). After chain assembly of the D-polypeptides was complete and the final Fmoc group removed, the resulting D-peptides had their side-chains deprotected and were simultaneously cleaved from the resin support by treatment with TFA containing 2.5% triisopropylsilane and 2.5% H₂O for 2.5 h at room temperature. Crude D-polypeptide products were recovered from resin by filtration and washing with cool ether, precipitated, and triturated with chilled diethyl ether then dried under vacuum. D-polypeptide chains folded spontaneously upon dissolution in appropriate buffer to yield the functional D-protein binder molecules.

Synthesis of the RFX-979820 D-Protein Construct

Step 1: Preparation of D-Cys-RFX-977296 Resin. Fmoc-aminoacyl-Rink Amide MBHA Resin was swelled in DMF (10-15 mL/g resin) for 1 h. The suspension was filtered, exchanged into DMF containing 20% piperidine, and kept at room temperature for 0.5 h under continuous nitrogen gas perfusion. The resin was then washed 5 times with DMF. For coupling, a pre-activated solution of Fmoc-D-amino acid-OH, DIC, HOBt and DMF was added to the resin. The suspension was kept at room temperature for 1 h while a stream of nitrogen was bubbled through it. The ninhydrin test was used to monitor the coupling reaction until completion. The remaining D-amino acids corresponding to the affinity matured D-protein RFX-977296 monomer were coupled to the peptidyl-resin sequentially. After assembly of the amino acid sequence of the protected RFX-977296 polypeptide chain was complete, the final Fmoc group was removed by treatment with DMF containing 20% piperidine, and Fmoc-D-Cys(Trt)-COOH was coupled to the N-terminus of the polypeptide chain. The Fmoc group was removed by treatment with DMF containing 20% piperidine, and the peptidyl-resin was washed with DMF (5 times), MeOH (2 times), DCM (2 times) and MeOH (2 times), then dried under vacuum overnight.

Step 2: Deprotection, Cleavage, and Purification of D-Cys-RFX-977296 resin. Cleavage solution (TFA/Thioanisole/phenol/EDT/H₂O=87.5/5/2.5/2.5/2.5 v/v, 60 mL) was added to the dried D-peptidyl-resin. The suspension was shaken for 3 h under N₂ and was filtered and the filtrate collected. Cold ether (10 eq.) was added to the filtrate to precipitate the peptide which was recovered by centrifugation. The white precipitate was washed with 10 eq. of ether twice, then dried under vacuum overnight to give crude D-peptide as a white solid. Purification of crude D-peptide was performed on a CXTH LC6000/Hanbon NU3000 prep system on a Phenomenex P227 C18 silica column (21.2×250 mm). Crude peptides were loaded onto the prep column and eluted at a flow rate of 60 mL/min with a shallow gradient of increasing concentrations of solvent B (0.1% TFA in 80% acetonitrile in water) in solvent A (0.1% TFA in water). Fractions containing the pure target peptide were identified by analytical LC-MS and then combined and lyophilized to give purified D-Cys-RFX-977296.

Step 3: Preparation, Cleavage, and Deprotection of D-Cys-RFX-978064 Resin. Fmoc-aminoacyl-Rink Amide MBHA Resin was prepared in the same manner as in Step 1. D-amino acids corresponding to the affinity matured D-protein RFX-978064 monomer were again coupled to the peptidyl resin sequentially, and peptide coupling and deprotection of D-Cys-RFX-978064 was carried out in the exact same manner as in Step 1. Cleavage of this monomer from the resin was performed in a separate cleavage solution (TFA/Thioanisole/phenol/EDT/H₂O=87.5/5/2.5/2.5/2.5 v/v, 60 mL), and purification was performed exactly as in Step 2.

Step 4: Preparation ofsingle modified Bis-Mal-PEG₆-D-Cys-RFX-978064. To a stirred solution of Bis-Mal-PEG₆ in PBS buffer (pH=7.4) was added dropwise a solution of D-Cys-RFX-978064 over 2 min, the reaction mixture was stirred at room temperature for 1 h, then the reaction mixture was purified by preparation of HPLC and lyophilized to give purified single modified Bis-Mal-PEG₆-D-Cys-RFX-978064.

Step 5: Preparation of RFX-979820. A stirred solution of single modified Bis-Mal-PEG₆-D-Cys-RFX-978064 (22 mg) and D-Cys-RFX-977296 (20.5 mg) in ACN/H₂O (V/V, 1:3, 2 mL), then PBS buffer (pH=7.4, 0.5 mL) was added to the reaction mixture and the reaction mixture was stirred at room temperature for 1 h. The reaction mixture was loaded onto a RP-HPLC without further workup and purified by gradient elution as described above. Fractions containing the desired product were identified by LCMS, combined, and lyophilized to give the D-protein construct (RFX-982007). The observed mass for RFX-979820 (LC-MS)=13,446.0+/−2 Da; the calculated mass (average isotope composition)=13,447 Da.

Synthesis of the Three Component RFX-982007D-Protein Construct

Step 1: Preparation of propargyl-PEG₃-D-RFX-979261 Resin. Fmoc-aminoacyl-Rink Amide MBHA Resin was swelled in DMF (10-15 mL/g resin) for 1 h. The suspension was filtered, exchanged into DMF containing 20% piperidine, and kept at room temperature for 0.5 h under continuous nitrogen gas perfusion. The resin was then washed 5 times with DMF. A pre-mixed solution of Fmoc-D-amino acid-OH, DIC, HOBt and DMF were added to the resin. The suspension was kept at room temperature for 1 h while a stream of nitrogen was bubbled through it. The ninhydrin test was used to monitor the coupling reaction until completion. The remaining D-amino acids corresponding to the affinity matured D-protein RFX-979261 monomer were coupled to the peptidyl resin sequentially. After assembly of the amino acid sequence of the protected D-RFX-979261 polypeptide chain was complete, the final Fmoc group was removed by treatment with DMF containing 20% piperidine, and Fmoc-D-propargyl-PEG₃-COOH was coupled to the N-terminus of the polypeptide chain. The peptidyl-resin was washed with DMF (5 times), MeOH (2 times), DCM (2 times) and MeOH (2 times), then dried under vacuum overnight.

Step 2: Cleavage, Deprotection, and Purification of propargyl-PEG₃-D-RFX-979261. Cleavage solution (TFA/Triisopropylsilane/H₂O=95/2.5/2.5 v/v, 60 mL) was added to the dried propargyl-PEG₃-D-RFX-979261-resin. The suspension was shaken for 2.5 h under N₂ and was filtered and the filtrate collected. Cold ether (10 eq.) was added to the filtrate to precipitate the peptide which was recovered by centrifugation. The white precipitate was washed with 10 eq. of ether twice, then dried under vacuum overnight to give crude propargyl-PEG₃-D-RFX-979261 as a white solid. Purification of crude propargyl-PEG₃-D-RFX-979261 was performed on a CXTH LC6000/Hanbon NU3000 prep system on a Phenomenex P227 C18 silica column. Crude peptide was loaded onto the prep column and eluted at a flow rate of 60 mL/min with a shallow gradient of increasing concentrations of solvent B (0.1% TFA in 80% acetonitrile in water) in solvent A (0.1% TFA in water). Fractions containing the pure target peptide were identified by analytical LC-MS and then combined and lyophilized to give purified propargyl-PEG₃-D-RFX-979261.

Step 3: Preparation, Cleavage, and Deprotection of D-979261 Resin. Fmoc-aminoacyl-Rink Amide MBHA Resin was prepared in the same manner as in Step 1. Fmoc-D-amino acids corresponding to the sequence of the affinity matured D-protein RFX-979261 polypeptide chain were coupled to the peptidyl resin sequentially. Fmoc-D-amino acid additions, removal of the final Fmoc group were carried out in the same manner as in Step 1. Deprotection and cleavage of D-RFX-979261 from the resin was performed in a cleavage solution consisting of TFA/thioanisole/phenol/EDT/H₂O 87.5/5/2.5/2.5/2.5 v/v, and purification was performed as in Step 2.

Step 4: Preparation of Azidoacetyl-PEG₃-D-RFX-977296. Fmoc-aminoacyl-Rink Amide MBHA Resin was prepared in the same manner as in Step 1. Fmoc-D-amino acids corresponding to the amino acid sequence of the D-protein RFX-977296 polypeptide chain were coupled to the peptidyl-resin sequentially. Fmoc-D-amino acid additions and removal of the final Fmoc group of RFX-977296 were carried out in the same manner as in Step 1. Deprotection and cleavage of RFX-977296 from the resin was performed in a solution consisting of TFA/thioanisole/phenol/EDT/H₂O 87.5/5/2.5/2.5/2.5 v/v, and purification was performed as in Step 2.

Step 5: Preparation of the Alkynyl-PEG₃-D-RFX-979261 (—S—S—) D-RFX-979261 two polypeptide chain construct. D-RFX-979261 and DTNP were dissolved in DMF with stirring. DIEA was then added, and the reaction was stirred at room temperature for 1.5 h under N₂. The reaction was concentrated and purified on a P1476 C18 column. The purified product was dissolved in a 1:1 solution of acetonitrile/H₂O (3 mL), and then 1.5 mL of PBS (0.1 M, pH=7.2) was added followed by a solution of alkynyl-PEG₃-D-RFX-979261 in acetonitrile/H₂O. The reaction mixture was stirred at room temperature under N₂until the disulfide-linked product was completely formed as shown by analytical LCMS. The crude product was purified on a P991 C18 column at a flow rate of 10 mL/min under the same buffer conditions as in Step 2.

Step 6: Click Reaction and Purification. Azidoacetyl-PEG₃-D-RFX-977296 and the Alkynyl-PEG₃-D-RFX-979261 (—S—S—) D-RFX-979261 construct were dissolved in an ethanol:H₂O solution (1:1 v/v). 0.12 mM CuSO₄ in H₂O was then added to the reaction mixture, followed by the addition of 0.12 mM of aqueous sodium ascorbate, and the reaction mixture was stirred at 30° C. for 2 h. The reaction mixture was loaded onto a RP-HPLC without further workup and purified by gradient elution as described above. Fractions containing the desired triazole-linked product were identified by LCMS, combined, and lyophilized to give the three component RFX-982007 D-protein construct. The observed mass for RFX-982007 (LC-MS) was 19,609.2+/−2 Da; calculated mass (average isotope composition) 19,612 Da.

### LC-MS Analysis of D-proteins

Analytical RP-HPLC was performed on a HP 1090 system with Waters C4/Phenomenex C18 silica columns (4.6×150 mm, 3.5 μm/4.6×150 mm, 5.0 μm particle size) at a flow rate of 1.0 mL/min (50° C. column temperature). Peptides were eluted from the column using a 1.0% B/min gradient of water/0.1% TFA (solvent A) versus 80% acetonitrile in water/0.1% TFA (solvent B). Peptide masses were obtained by in-line electrospray MS detection using an Agilent 6120 LC/MSD ion trap.

Surface Plasmon Resonance Affinity Measurements

Surface plasmon resonance (SPR) binding measurements were carried out on a Biacore S200 (GE). Biotinylated PD-1-Fc fusion protein was immobilized on a streptavidin chip (GE) using a concentration of 5 gg/mL at a flow rate of 5 μl/min for 400 seconds. Titrations of D-proteins were carried out using 2-fold serial dilutions flowed over the chip at 30 μL/min in running buffer (10 mM Hepes, pH 7.4, 150 mM NaCl, 0.05% P20) with a max concentration of either 2 μM (RFX-978064 and -977296) or 100 nM (RFX-979261). Association time was 120 seconds followed by a 240 second dissociation. Given the very high affinities of nivolumab, RFX-979820, and RFX-982007, single-cycle kinetic experiments were carried out using 2-fold serial dilutions starting from 50 nM with association time of 200 seconds for each injection followed by final dissociation for 3600 seconds. All measurements were carried out at 25° C. SPR data are representative of multiple independent titrations. Kinetic fits were performed using Biacore software using a global single site binding model.

Expression and Purification of PD-1 for Crystallography

The gene sequence for the PD-1 (25-167) polypeptide chain was cloned into the expression vector pET21b with a 6×His tag and TEV cleavage site added at the N-terminus. The recombinant plasmid was transformed into E. coli BL21-Gold, grown in LB medium supplemented with Ampicillin (100 μg/ml) and expression of the His-tagged protein was induced by 0.3 mM isopropyl-β-D-thiogalactoside (IPTG) at 16° C. overnight. Cells were harvested by centrifugation and then stored at −80° C.

Pelleted cells from 30 L of culture were resuspended in 1 L buffer A (20 mM Tris, pH 8.0, 400 mM NaCl) and then passed through high-pressure homogenization (3 cycles). His-tagged protein from supernatant was captured on a Ni-NTA resin column (30 ml). The column was washed with 20 C.V. of Buffer A containing 20 mM imidazole, 5 CV of Buffer C (20 mM Tris, pH 8.0, 1M NaCl) and 10 CV of buffer A containing 50 mM imidazole. The 6×His-tagged PD-1 protein was eluted with a high concentration of imidazole (0.25 M) in buffer A (5 C.V.). The eluted protein was digested with TEV protease at a 1:20 ratio (TEV:Protein) and dialyzed against 5 L buffer (20 mM Tris, pH 8.0, 50 mM NaCl) at 4° C. overnight. Cleaved sample was loaded onto a 2^{nd} Ni-NTA column to remove free His-tag and buffer exchanged into SEC buffer (10 mM Tris-HCl pH 8.0, 20 mM NaCl). A final SEC polishing step was performed using a Superdex 75 10/300 GL column equilibrated with SEC buffer. Monodisperse PD-1 peak fractions were identified by absorbance at 280 nm and were combined and concentrated to 12.1 mg/mL in SEC buffer. Final purified PD-1 (25-167) protein was 80% pure as assessed by SDS-PAGE analysis and the molecular weight was confirmed by direct injection MS.

Crystallography of PD-1/D-Protein Triple Complex

Crystals for the PD-1/RFX-977296/RFX-978064 complex were grown by hanging drop vapor diffusion at 18° C. The drop was composed of 0.5 μL of PD-1/D-protein complex (5.0 mg/ml PD-1, 270 M RFX-978064, and 270 M RFX-977296) mixed 1:1 with 0.51 of the crystallization solution containing 0.2 M ammonium acetate, 0.1 M Bis-Tris pH 5.5, 25% w/v PEG 3350. The diffraction data were collected at the Shanghai Synchrotron Radiation Facility beam line BL19U1 to 2.46 Angstroms resolution and processed in space group P41212 using XDS. The structure was solved by molecular replacement using Phaser with PD-1 structure (PDB ID: 3RRQ) as the search model. Structure refinement and model building on the initial model were performed using Refmac5. There is one copy of PD-1, one copy of RFX-978064, and one copy of RFX-977296 in an asymmetric unit. The detailed data processing and structure refinement statistics are listed in Table S3. All structural images were rendered using Pymol (Schrodinger).

PD-1 PD-LI Binding ELISAs

Human PD-1-Fc was purchased from R&D Systems (cat #1086-PD-050) and biotinylated using sulfo-NHS-LC-LC-biotin (Pierce, cat # A35358) according to manufacturer's protocol. PD-L1-Fc was purchased from R&D Systems (cat #156-B7-100). Nivolumab was manufactured by Bristol Myers Squibb (lot # AAYi999). In all cases, 1 gg/mL of PD-L1-Fc or nivolmab was coated on MaxiSorp plates overnight at 4° C. The following day, coated wells were washed with PBS-T (1×PBS+0.01% Tween 20) and blocked with Super Block (Rockland) for 2 h with shaking at room temp. For ELISAs measuring binding for PD-1-Fc to PD-L1-Fc, titrations of the D-proteins and nivolumab were incubated with 4.0 nM of biotinylated PD-1-Fc for 60 min before addition to blocked PD-L1-Fc coated wells. For ELISAs measuring binding for PD-1-Fc to nivolumab, titrations of the D-proteins and nivolumab were incubated with 0.5 nM of biotinylated PD-1-Fc for 60 min before addition to blocked nivolumab coated wells. The antagonist/PD-1-Fc mixtures were then incubated on PD-L1-Fc or nivolumab coated wells for 1 h with shaking at room temp, washed 3 times with wash buffer (PBS, 0.05% Tween 20), and bound biotinylated PD-1-Fc was detected with streptavidin-HRP (ThermoFisher, cat # N-100). Data plotted are mean±standard deviation of triplicate experiments. IC₅₀ values were derived from 3-parameter fits using Prism (GraphPad) and the error reported is derived from fits.

PD-1 Blockade Assay

Measurement of PD-1/PD-L1 inhibition was performed using the PD-1/PD-L1 Blockade Bioassay (Promega, cat # J1250). Briefly, Jurkat T cells are engineered to stably express human PD-1 and a T-cell receptor (TCR) signaling reporter system composed of a NFAT-inducible luciferase response element. Activated Jurkat T-cells express high levels of luciferase, which is inhibited when co-cultured with artificial APCs stably expressing PD-L1 to mimic T-cell exhaustion and suppression of TCR signaling. PD-1/PD-L1 blockade relieves suppression of TCR signaling and restores luciferase expression, which can be quantified using bioluminescence. The engineered Jurkat T-cells were titrated with D-protein or nivolumab PD-1 antagonists, mixed with artificial APCs and incubated at 37° C., 5% CO₂ for 6 hours. Following incubation, Bio-Glo was added to wells according to the manufacturer's protocol and relative luminescence units (RLUs) were measured on a PerkinElmer 2300 Enspire Multimode plate reader. Data plotted are mean±standard deviation of triplicate measurements. IC₅₀ values were derived from 3-parameter fits using Prism (GraphPad) and error reported are derived from fits.

CMV Recall Assay

Cytokine production from total human PBMCs was measured following stimulation with CMV antigens. Briefly, 2.5×10⁵ PBMCs isolated from a CMV-positive donor were labeled with 2.5 μM CFSE, washed, and stimulated with CMV antigen lysate at 1 gg/mL (Astarte, cat #1004) plus 10 U/ml human IL-2 and in the absence or presence of PD-1 antagonist titrations. Stimulated PBMCs were incubated in 96-well round bottom plates for 4 days at 37° C., 5% CO₂. Following incubation, tissue culture supernatant was collected and analyzed for IFN-γ and TNF-α using a flow cytometry-based cytometric bead array (MultiCyt Qbeads Plexscreen, Intellicyt) while CD8⁺ T-cell proliferation was measured using flow cytometry to assess CFSE dilution. For flow cytometry of CD8⁺ T-cell proliferation, PBMCs were stained with an anti-CD8 antibody (clone RPA-T8-APC, BioLegend cat #301049) and CFSE dilution was measured for this population. All flow cytometry was performed on an Intellicyt iQue Screener Plus and analysis was carried out using ForeCyt software. Data plotted are mean±SEM of triplicate measurements.

Subcutaneous Immunization in BALB c Mice

Adjuvant was purchased from TiterMax. Female BALB/c mice (6-8 weeks) were randomized into immunization groups on Day 0 (n=5 per group). Immunizations were performed on Days 0, 21, 35 by subcutaneous injection of 25 gg of antigen (nivolumab or RFX-982007). Antigens were emulsified in adjuvant for injection on Day 0 and administered in PBS for Days 21 and 35. Serum pre-bleeds were performed on Days 0, 21, 35 prior to immunizations. Final bleeds for max titer response were taken on Day 42. All the procedures related to animal handling, care and treatment in the study were performed according to the guidelines set forth in an ACUP protocol for polyclonal antisera production in mice, approved by the Institutional Animal Care and Use Committee (IACUC) of Josman LLC.

### REFERENCES

1. S. C. Wei, C. R. Duffy, J. P. Allison, Fundamental Mechanisms of Immune Checkpoint Blockade Therapy. Cancer Discov. 8, 1069-1086 (2018).
2. N. Lonberg, A. J. Korman, Masterful Antibodies: Checkpoint Blockade. Cancer Immunol. Res. 5, 275-281 (2017).
3. S. Terawaki et al., Specific and high-affinity binding of tetramerized PD-L1 extracellular domain to PD-1-expressing cells: possible application to enhance T cell function. Int. Immunol. 19, 881-890 (2007).
4. R. M. Wong et al., Programmed death-1 blockade enhances expansion and functional capacity of human melanoma antigen-specific CTLs. Int. Immunol. 19, 1223-1234 (2007).
5. J. R. Brahmer et al., Phase I study of single-agent anti-programmed death-1 (MDX-1106) in refractory solid tumors: safety, clinical activity, pharmacodynamics, and immunologic correlates. J. Clin. Oncol. 28, 3167-3175 (2010).
6. A. Haslam, V. Prasad, Estimation of the Percentage of US Patients With Cancer Who Are Eligible for and Respond to Checkpoint Inhibitor Immunotherapy Drugs. JAMA Netw. open. 2, e192535 (2019).
7. R. L. Maute et al., Engineering high-affinity PD-1 variants for optimized immunotherapy and immuno-PET imaging. Proc. Natl. Acad. Sci. U.S.A 112, E6506-14 (2015).
8. M. Centanni, D. J. A. R. Moes, I. F. Troconiz, J. Ciccolini, J. G. C. van Hasselt, Clinical Pharmacokinetics and Pharmacodynamics of Immune Checkpoint Inhibitors. Clin. Pharmacokinet. 58, 835-857 (2019).
9. M. A. Couey et al., Delayed immune-related events (DIRE) after discontinuation of immunotherapy: diagnostic hazard of autoimmunity at a distance. J. Immunother. cancer. 7, 165 (2019).
10. J. Davda et al., Immunogenicity of immunomodulatory, antibody-based, oncology therapeutics. J. Immunother. cancer. 7, 105 (2019).
11. K. Guzik et al., Development of the Inhibitors that Target the PD-1/PD-L1 Interaction-A Brief Look at Progress on Small Molecules, Peptides and Macrocycles. Molecules. 24 (2019).
12. M. Uppalapati et al., A Potent D-Protein Antagonist of VEGF-A is Nonimmunogenic, Metabolically Stable, and Longer-Circulating in Vivo. ACS Chem. Biol. 11, 1058-1065 (2016).
13. H. M. Dintzis, D. E. Symer, R. Z. Dintzis, L. E. Zawadzke, J. M. Berg, A comparison of the immunogenicity of a pair of enantiomeric proteins. Proteins. 16, 306-308 (1993).
14. P. S. Marinec et al., A Synthetic D-Protein Durably Blocks Retinal Vascularization and Inhibits Tumor Growth. Science. X, X-X (2020).
15. T. N. Schumacher et al., Identification of D-peptide ligands through mirror-image phage display. Science. 271, 1854-1857 (1996).
16. P. E. Dawson, T. W. Muir, I. Clark-Lewis, S. B. Kent, Synthesis of proteins by native chemical ligation. Science. 266, 776-779 (1994).
17. S. B. H. Kent, Novel protein science enabled by total chemical synthesis. Protein Sci. 28, 313-328 (2019).
18. D. M. Pardoll, The blockade of immune checkpoints in cancer immunotherapy. Nat. Rev. Cancer. 12, 252-264 (2012).
19. C. Wang et al., In vitro characterization of the anti-PD-1 antibody nivolumab, BMS—936558, and in vivo toxicology in non-human primates. Cancer Immunol. Res. 2, 846-856 (2014).
20. M. Tashiro et al., High-resolution solution NMR structure of the Z domain of staphylococcal protein A. J. Mol. Biol. 272, 573-590 (1997).
21. S. Lejon, I.-M. Frick, L. Bjorck, M. Wikstrom, S. Svensson, Crystal structure and biological implications of a bacterial albumin binding module in complex with human serum albumin. J. Biol. Chem. 279, 42924-42928 (2004).
22. K. M. Zak et al., Structure of the Complex of Human Programmed Death 1, PD-1, and Its Ligand PD-L1. Structure. 23, 2341-2348 (2015).
23. S. L. Topalian et al., Safety, activity, and immune correlates of anti-PD-1 antibody in cancer. N. Engl. J. Med. 366, 2443-2454 (2012).
24. A. M. M. Eggermont et al., Adjuvant Pembrolizumab versus Placebo in Resected Stage III Melanoma. N. Engl. J. Med. 378, 1789-1801 (2018).
25. M. R. Migden et al., PD-1 Blockade with Cemiplimab in Advanced Cutaneous Squamous-Cell Carcinoma. N. Engl. J. Med. 379, 341-351 (2018).
26. M. A. Socinski et al., Atezolizumab for First-Line Treatment of Metastatic Nonsquamous NSCLC. N. Engl. J. Med. 378, 2288-2301 (2018).
27. H. L. Kaufman et al., Avelumab in patients with chemotherapy-refractory metastatic Merkel cell carcinoma: a multicentre, single-group, open-label, phase 2 trial. Lancet. Oncol. 17, 1374-1385 (2016).
28. S. J. Antonia et al., Durvalumab after Chemoradiotherapy in Stage III Non-Small-Cell Lung Cancer. N. Engl. J. Med. 377, 1919-1929 (2017).
29. P. Fessas, H. Lee, S. Ikemizu, T. Janowitz, A molecular and preclinical comparison of the PD-1-targeted T-cell checkpoint inhibitors nivolumab and pembrolizumab. Semin. Oncol. 44, 136-140 (2017).
30. J. Powderly et al., CA-170, a first in class oral small molecule dual inhibitor of immune checkpoints PD-L1 and VISTA, demonstrates tumor growth inhibition in pre-clinical models and promotes T cell activation in Phase 1 study. Ann. Oncol. 28, v403-v427 (2017).
31. B. Musielak et al., CA-170—A Potent Small-Molecule PD-L1 Inhibitor or Not? Molecules. 24 (2019).
32. X. Cheng et al., Structure and interactions of the human programmed cell death 1 receptor. J. Biol. Chem. 288, 11771-11785 (2013).
33. S. Tang, P. S. Kim, A high-affinity human PD-1/PD-L2 complex informs avenues for small-molecule immune checkpoint drug discovery. Proc. Natl. Acad. Sci. U.S.A 116, 24500-24506 (2019).
34. J. Y. Lee et al., Structural basis of checkpoint blockade by monoclonal antibodies in cancer immunotherapy. Nat. Commun. 7, 13354 (2016).
35. C. Fenwick et al., Tumor suppression of novel anti-PD-1 antibodies mediated through CD28 costimulatory pathway. J. Exp. Med. 216, 1525-1541 (2019).
36. E. Hui et al., T cell costimulatory receptor CD28 is a primary target for PD-1-mediated inhibition. Science. 355, 1428-1433 (2017).
37. M. Gumbleton et al., Dual enhancement of T and NK cell function by pulsatile inhibition of SHIP1 improves antitumor immunity and survival. Sci. Signal. 10 (2017).
38. H. Choi et al., Pulsatile MEK Inhibition Improves Anti-tumor Immunity and T Cell Function in Murine Kras Mutant Lung Cancer. Cell Rep. 27, 806-819.e5 (2019).
39. A. Cheng et al., LBA3-IMbrave150: Efficacy and safety results from a ph III study evaluating atezolizumab+bevacizumab vs sorafenib as first treatment for patients with unresectable hepatocellular carcinoma. Ann. Oncol. 30, ix183-ix202 (2019).
40. D. F. McDermott et al., Clinical activity and molecular correlates of response to atezolizumab alone or in combination with bevacizumab versus sunitinib in renal cell carcinoma. Nat. Med. 24, 749-757 (2018).
41. J. J. Wallin et al., Atezolizumab in combination with bevacizumab enhances antigen-specific T-cell migration in metastatic renal cell carcinoma. Nat. Commun. 7, 12624 (2016).
42. S. S. Sidhu, B. K. Feld, G. A. Weiss, M13 Bacteriophage Coat Proteins Engineered for Improved Phage Display. Protein Eng. Protoc., 205-220 (2006).
43. T. A. Kunkel, Rapid and efficient site-specific mutagenesis without phenotypic selection. Proc. Natl. Acad. Sci. 82, 488-492 (1985).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1-76. (canceled)

77. A D-protein compound, comprising:
(a) a first D-domain that specifically binds a target protein at a first binding site; and
(b) a second D-domain that specifically binds the target protein at a second binding site; and
(c) a linker configured to connect the first and second D-domains whereby the D-domains are capable of simultaneously binding the target protein.

78. The compound of claim 77, wherein the compound is bivalent and has a target protein binding affinity that is at least 10-fold stronger than the target protein binding affinity of a monovalent first D-domain and of a monovalent second D-domain.

79. The compound of claim 78, wherein the compound has a target protein binding affinity (K_{D}) that is 3 nM or less, as measured by SPR.

80. The compound of claim 77, wherein the first D-domain specifically binds an antagonist binding site of a target protein.

81. The compound of claim 77, wherein the compound is dimeric.

82. The compound of claim 77, further comprising a third D-domain that specifically binds the target protein whereby the compound is trimeric.

83. The compound of claim 77, wherein the compound is multispecific.

84. The compound of claim 83, wherein the compound is bispecific.

85. The compound of claim 77, wherein the first and second D-domains are heterologous scaffold domains.

86. The D-peptidic compound of claim 85, wherein the third and first D-domains are homologous scaffold domains.

87. The compound of claim 77, wherein the D-domains each independently comprise a single chain D-polypeptide sequence having 30 to 80 residues.

88. The compound of claim 87, wherein each D-domain is a three-helix bundle domain.

89. The compound of claim 88, wherein each D-domain is independently selected from a GA domain, a Z domain, and an albumin-binding domain (ABD).

90. The compound of claim 88, wherein one or more of the D-domains comprises an interhelix linker.

91. The compound of claim 77, wherein each D-domain has a specificity-determining motif (SDM) comprising 5 or more variant amino acid residues located at the target-binding face of the D-domain.

92. The compound of claim 91, wherein each SDM comprises 10 or more variant amino acid residues.

93. The compound of claim 77, wherein the linker is a peptidic linker.

94. The compound of claim 77, wherein the linker is a non-peptidic linker.

95. The compound of claim 77, wherein the linker connects the first and second D-domains via amino acid residues that are proximal to each other when the D-domains are simultaneously bound to the target protein.

96. The compound of claim 95, wherein the linker connects the proximal amino acid residues via their sidechain groups.

97. The compound of claim 95, wherein the linker connects the proximal amino acid residues via their N-terminal and/or C-terminal groups.

98. The compound of claim 95, wherein the linker connects the proximal amino acid residues via connection from one terminal group to one sidechain group.

99. The compound of claim 94, wherein the linker comprises one or more linking groups selected from amino acid residue, polypeptide, (PEG)ₙ linker, modified PEG moiety, C₍₁₋₆₎alkyl linker, substituted C₍₁₋₆₎alkyl linker, —CO(CH₂)ₘCO—, —NR(CH₂)ₚNR—, —CO(CH₂)ₘNR—, —CO(CH₂)ₘO—, —CO(CH₂)ₘS—, and linked chemoselective functional groups, wherein m is 1 to 6, p is 2-6 and each R is independently H, C₍₁₋₆₎alkyl or substituted C₍₁₋₆₎alkyl.

100. The compound of claim 77, wherein the compound is thermostable and has a melt temperature of 50° C. or more.

101. The compound of claim 77, wherein the compound has an in vitro half-life in human serum of 12 hours or longer.

102. The compound of claim 77, wherein the compound is non-immunogenic.

103. A D-protein compound, comprising a D-domain that specifically binds a first target protein and antagonizes the first target protein in an in vitro cell based activity assay.

104. The compound of claim 103, wherein the D-domain has a scaffold domain that is a three-helix bundle domain.

105. The compound of claim 104, wherein each D-domain is independently selected from a GA domain, a Z domain, and an albumin-binding domain (ABD).

106. The compound of claim 103, wherein the D-domains each independently comprise a single chain D-polypeptide sequence having 30 to 80 residues.

107. The compound of claim 103, wherein the compound is monomeric.

108. The compound of claim 103, wherein the compound is homodimeric.

109. The compound of claim 103, wherein the compound is heterodimeric.

110. The compound of claim 109, wherein the compound is bispecific whereby the compound further comprises a second D-domain that specifically binds a second target protein.

111. The compound of claim 103, wherein the compound has a target protein binding affinity (K_{D}) of 10 nM or less.
